(19) United States
(12) Patent Application Publication
(10) Pub. No.: US 2010/0249179 A1
(43) Pub. Date: Sep. 30, 2010

(54) Farnesoid X Receptor Agonists

(75) Inventors: David Norman Deaton, Durham, NC (US); Frank Navas, III, Durham, NC (US); Paul Kenneth Spearing, Durham, NC (US)

Correspondence Address: GLAXOSMITHKLINE;GLOBAL PATENTS, C.2111F, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 12/663,722

(22) PCT Filed: Jun. 13, 2008
(86) PCT No.: PCT/US2008/066800

§ 371 Date: Jun. 15, 2010

Related U.S. Application Data
(60) Provisional application No. 60/943,573, filed on Jun. 13, 2007.

(51) Int. Cl. A61K 31/4365; C07D 497/04; A61P 1/16; C07D 413/12; A61K 31/422; C07D 413/14; A61K 31/4439; A61K 31/423

(52) U.S. Cl. 514/301; 546/114; 548/247; 514/378; 546/271.1; 514/339; 548/241; 514/379

(57) ABSTRACT

The present invention provides novel isoxazole compounds, pharmaceutical compositions, therapeutic uses and processes for preparing the same.

### BACKGROUND OF THE INVENTION

The present invention relates to farnesoid X receptors (FXR, NR1H4). More particularly, the present invention relates to compounds useful as agonists for FXR, pharmaceutical formulations comprising such compounds, and therapeutic use of the same.

FXR is a member of the nuclear receptor class of ligand-activated transcription factors. Physiological concentrations of bile acids bind and activate FXR. [Parks, D. J., et al. 1999 Science 284:1365-1368; Makishima, M., et al. 1999 Science 284:1362-1365] Bile acids are amphipathic molecules that form micelles and emulsify dietary lipids. This property also makes bile acids cytotoxic if sufficient concentrations are achieved and thus mechanisms have evolved to ensure bile acid concentrations are tightly regulated. FXR plays a key role in regulating bile acid homeostasis. [Makishima, M. 2005 J. Pharmacol. Sci. 97:177-183; Kuipers, F., et al. Rev. Endocrine Metab. Disorders 5:319-326]

FXR is expressed in liver, intestine, kidney, and adrenal. [Kuipers, F., et al. 2004 Rev. Endocrine Metab. Disorders 5:319-326] FXR target genes in hepatocytes include small heterodimer partner (SHP, NR0B2) which encodes an atypical nuclear receptor that represses transcription of genes such as CYP7A1 (encoding cholesterol 7α-hydroxylase), the first and rate limiting step in the conversion of cholesterol to bile acid, CYP8B1 (encoding sterol 12α-hydroxylase) which controls the hydrophobicity of the bile pool and NTCP (encoding the sodium/taurocholate co-transporting polypeptide, SLC10A1) that imports bile acids from the portal and systemic circulation into the hepatocyte. [Goodwin, B., et al. 2000 Mol. Cell. 6:517-526; del Castillo-Olivares, A., et al 2001 Nucleic Acids Res. 29:4035-4042; Denson, L. A., et al. 2001 Gastroenterology 121(1):140-147] Other FXR target genes that are induced in liver include the canalicular transporter BSEP (encoding the bile salt export pump, ABCB11) that transports bile acids from the hepatocyte into the bile, multi-drug resistance P glycoprotein-3 (MDR3) (encoding the canalicular phospholipid flippase, ABCB4) that transports phospholipids from the hepatocyte into the bile and MRP2 (encoding multidrug resistance-related protein-2, ABCC2) that transports conjugated bilirubin, glutathione and glutathione conjugates into bile. [Ananthanarayanan, M., et al. 2001 J. Biol. Chem. 276:28857-28865; Huang, L., et al., 2003 J. Biol. Chem. 278:51085-51090; Kast, H. R., et al. 2002 J. Biol. Chem. 277:2908-2915.]

In the intestine, FXR also induces expression of SHP which represses transcription of the apical sodium dependent bile acid transporter (ASBT, SLC 10A2) gene which encodes the high affinity apical sodium dependent bile acid transporter that moves bile acids from the intestinal lumen into the enterocyte as part of the enterohepatic recycling of bile acids. [Li, H., et al. 2005 μm. J. Physiol. Gastrointest. Liver Physiol. 288:G60-G66] Ileal bile acid binding protein (IBABP) gene expression is also induced by FXR agonists in the enterocyte. [Grober, J., et al., 1999 J. Biol. Chem. 274:29749-29754] The function of this ileal bile acid binding protein remains under investigation.

Cholestasis is a condition of reduced or arrested bile flow. Unresolved cholestasis leads to liver damage such as that seen in primary biliary cirrhosis (PBC) and primary sclerosing cholangitis (PSC), two cholestatic liver diseases. FXR agonists have been shown to protect the liver in rodent models of cholestatic liver disease. [Liu, Y., et al. 2003 J. Clin. Invest. 112:1678-1687; Fiorucci, S., et al. 2005 J. Pharmacol. Exp. Ther. 313:604-612; Pellicciari, R., et al. 2002 J. Med. Chem. 45:3569-3572]

FXR is also expressed in hepatic stellate cells (HSC) which play a role in deposition of extracellular matrix during the fibrotic process. Treatment of cultured HSCs with the FXR agonist 6-ethyl-chenodeoxycholic acid (6EtCDCA) results in decreased expression of fibrotic markers such as α-smooth muscle actin and α1(I) collagen. 6EtCDCA has also been reported to prevent development and promote resolution of hepatic fibrosis in multiple rodent models of this disease. [Fiorucci, S., et al., 2004 Gastroenterology 127:1497-1512; Fiorucci, S., et al., 2005 J. Pharmacol. Exp. Ther. 314:584-595.] According to Fiorucci et al., this anti-fibrotic effect is due to SHP inactivation of Jun and subsequent repression of tissue inhibitor of metalloproteinase 1 (TIMP1) via the activation protein 1 (AP1) binding site on the TIMP1 promoter.

S. Kliewer presented data at the Digestive Diseases Week (DDW) Conference (2005) organized by the American Association for the study of Liver Disease (AASLD) showing that activation of FXR by the agonist GW4064 resulted in improved mucosal barrier and decreased bacterial overgrowth in a bile duct-ligated mouse model of cholestasis and intestinal bacterial overgrowth. Dr. Kliewer showed data indicating decreased translocation of bacteria to mesenteric lymph nodes in mice treated with GW4064. This effect of GW4064 was lost in FXR null mice. [Inagaki, T., et al. 2006 Proc. Nat. Acad. Sci., U.S.A. 103:3920-3925.]

The FXR agonist GW4064, when administered to mice on a lithogenic diet, prevented the formation of cholesterol crystals in the bile. This effect of the compound was lost in FXR null mice. Moschetta, A., et al. 2004 Nat. Med. 10:1352-1358.

It has been suggested that GW4064 could improve lipid and glucose homeostasis and insulin sensitivity in rodent diabetic and insulin resistance models. Chen and colleagues [2006 Diabetes 55 suppl. 1: A200] demonstrated that when administered to mice on high-fat diet, GW4064 decrease body weight and body fat mass, serum glucose, insulin, triglyceride, and total cholesterol. GW4064 also corrected glucose intolerance in those animals. In addition, GW4064 decreased serum insulin concentration, improved glucose tolerance and enhanced insulin sensitivity in ob/ob mice [Cariou, B., et al., 2006 J. Biol. Chem. 281:11039-11049]. In another study, it was reported that GW4064 significantly improved hyperglycemia and hyperlipidemia in diabetic db/db mice [Zhang, Y., et al. 2006 Proc. Nat. Acad. Sci., U.S.A. 103:1006-1011].

### SUMMARY OF THE INVENTION

As a first aspect, the present invention provides compounds of formula (I):

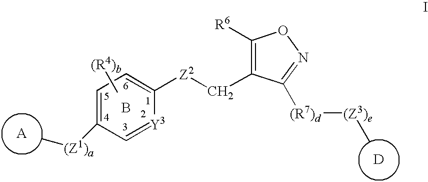

wherein:
Ring A is selected from

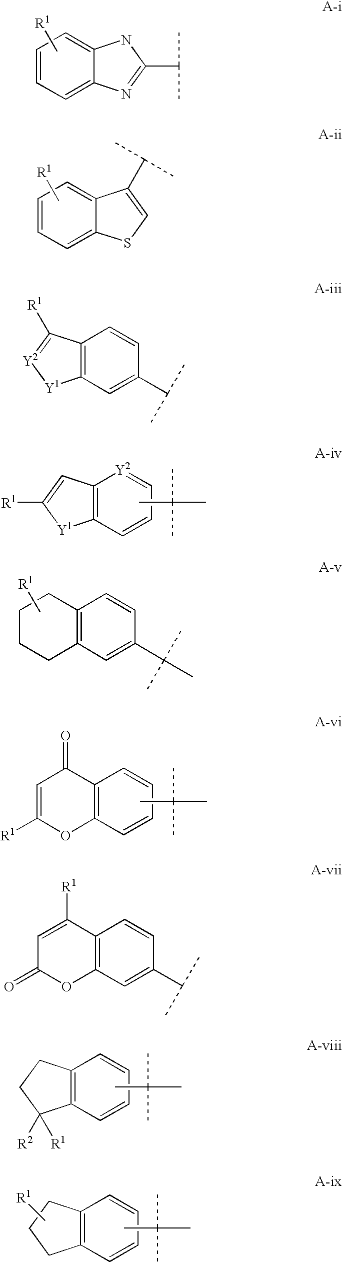

wherein
R¹ is selected from —CO₂H, —C(O)NH₂, —CO₂alkyl, and an acid equivalent group;
R² is H or —OH;
Y¹ is selected from —CH₂—, —NH—, —O— and —S—;
Y² is selected from —CH— and —N—; or
   Ring A is a substituted naphthalene when a=1;

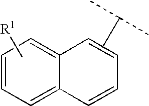

Z¹ is —NH— or —S—;
a is 0 or 1;
each R⁴ is selected from halo, alkyl and fluoroalkyl;
b is 0, 1 or 2, except that when b is 2 and Y³ is C, R⁴ is not bound at position 2 or 6 of Ring B;
Y³ is —N— or —CH—;
Z² is —O—, —S— or —N(R⁵)—, wherein R⁵ is H or alkyl;
R⁶ is selected from alkyl, 2,2,2-trifluoroethyl, C₃₋₆cycloalkyl, alkenyl, C₃₋₆cycloalkenyl and fluoro-substituted C₃₋₆cycloalkyl;
R⁷ is —C₁₋₃alkylene-;
Z³ is —O—, —S(O)_{c}—, or —NH—, where c is 0, 1 or 2;
d and e are both 0 or d is 1 and e is 0 or 1;
   Ring D is selected from C₃₋₆cycloalkyl and a moiety selected from formula D-i, D-ii and D-iii:

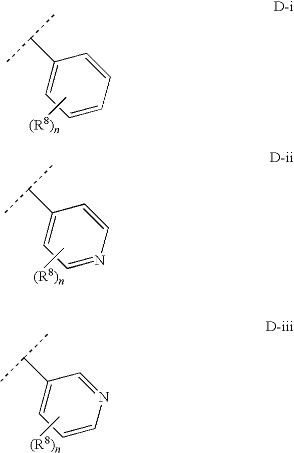

wherein
n is 0, 1, 2 or 3;
each R⁸ is the same or different and is independently selected from halo, alkyl, alkenyl, —O-alkyl, haloalkyl, —O-haloalkyl, hydroxyl substituted alkyl, and —OCF₃;
   and pharmaceutically acceptable salts thereof.

In a second aspect, the present invention provides a pharmaceutical composition comprising a compound of formula (I). The composition may further comprise a pharmaceutically acceptable carrier or diluent.

In a third aspect, the present invention provides a method for the treatment of a condition mediated by decreased FXR activity in a subject in need thereof. The method comprises administering to the subject a therapeutically effective amount of a compound of formula (I).

In a fourth aspect, the present invention provides a method for the treatment of obesity in a subject in need thereof. The method comprises administering to the subject a therapeutically effective amount of a compound of formula (I).

In a fifth aspect, the present invention provides a method for the treatment of diabetes mellitus in a subject in need thereof. The method comprises administering to the subject a therapeutically effective amount of a compound of formula (I).

In a sixth aspect, the present invention provides a method for the treatment of metabolic syndrome in a subject in need thereof. The method comprises administering to the subject a therapeutically effective amount of a compound of formula (I).

In a seventh aspect, the present invention provides a method for the treatment of cholestatic liver disease in a subject in need thereof. The method comprises administering to the subject a therapeutically effective amount of a compound of formula (I).

In a eighth aspect, the present invention provides a method for the treatment of organ fibrosis in a subject in need thereof. The method comprises administering to the subject a therapeutically effective amount of a compound of formula (I). In one embodiment, the organ fibrosis is liver fibrosis.

In a ninth aspect, the present invention provides a method for the treatment of liver fibrosis in a subject in need thereof. The method comprises administering to the subject a therapeutically effective amount of a compound of formula (I).

In a tenth aspect, the present invention provides a process for preparing a compound of formula (I). The process comprises the steps of:

a) reacting a compound of formula (II)

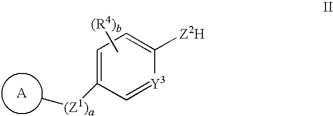

with a compound of formula (III)

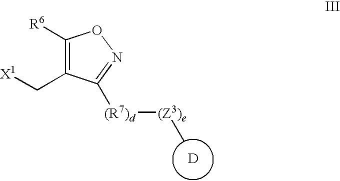

wherein X¹ is chloride, iodide, bromide, triflate, tosylate, nosylate, besylate or mesylate, (preferably chloro);
R¹ is —CO₂alkyl;
if A is A-viii, then R² is H; and
all other variables are as defined above for formula (I)
   to prepare a compound of formula (I); and
   b) optionally converting the compound of formula (I) into a different compound of formula (I).

In another aspect, the present invention provides another process for preparing a compound of formula (I). This process comprises the steps of:

a) reacting a compound of formula (II)

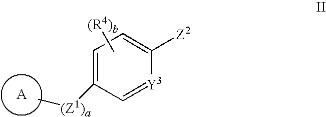

with a compound of formula (IV)

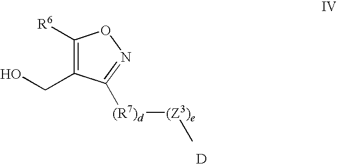

wherein
R¹ is —CO₂alkyl;
if A is A-viii, then R² is H; and
all other variables are as defined above for formula (I)
   to prepare a compound of formula (I); and
   b) optionally converting the compound of formula (I) into a different compound of formula (I).

In another aspect, the present invention provides another process for preparing a compound of formula (I). This process comprises the steps of:

a) reacting a compound of formula (XI)

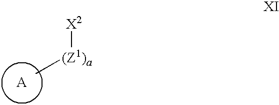

with a boronic acid or ester compound of formula (XLI) under Suzuki coupling conditions

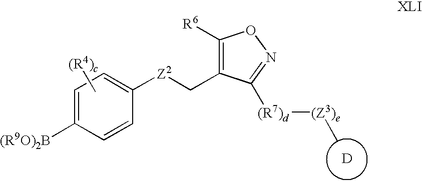

wherein
R¹ is —CO₂alkyl;
if A is A-viii, then R² is H;
a is 0;
X² is chloro, bromo, iodo, or triflate
R⁹ is H or alkyl; and
all other variables are as defined above for formula (I)
   to prepare a compound of formula (I); and
   b) optionally converting the compound of formula (I) into a different compound of formula (I).

In another aspect, the present invention provides another process for preparing a compound of formula (I). This process comprises the steps of:

a) reacting a compound of formula (IV) with a base to prepare an anion;

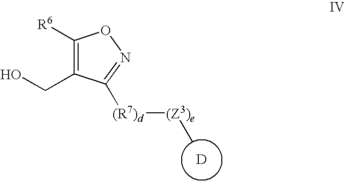

b) condensing the anion with a compound of formula (XLIII) to prepare a compound of formula (I);

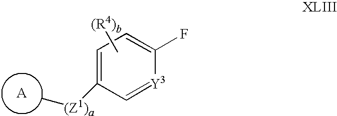

wherein
R¹ is —CO₂alkyl;
if A is A-viii, then R² is H;
Y³ is N; and
all other variables are as defined above for formula (I)
   to form a compound of formula (I); and
   c) optionally converting the compound of formula (I) into a different compound of formula (I).

In another aspect, the present invention provides another process for preparing a compound of formula (I). This process comprises the steps of:

a) reacting a compound of formula (II-g)

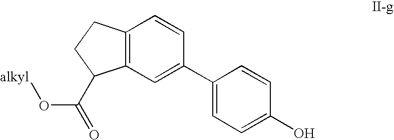

with a compound of formula (III)

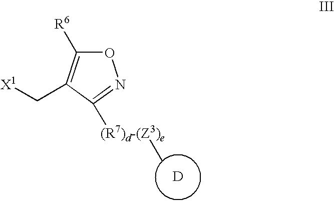

wherein X¹ is chloride; and
all other variables are as defined above for formula (I)
   to prepare a compound of formula (I); and
   b) optionally converting the compound of formula (I) into a different compound of formula (I).

In another aspect, the present invention provides a compound of formula (I) for use in therapy. The present invention also provides a compound of formula (I) for use in the treatment of a condition mediated by decreased FXR activity in a subject; a compound of formula (I) for use in the treatment of obesity in a subject; a compound of formula (I) for use in the treatment of diabetes mellitus in a subject; a compound of formula (I) for use in the treatment of metabolic syndrome in a subject; a compound of formula (I) for use in the treatment of cholestatic liver disease in a subject; a compound of formula (I) for use in the treatment of organ fibrosis in a subject; and a compound of formula (I) for use in the treatment of liver fibrosis in a subject.

In another aspect, the present invention provides the use of a compound of formula (I) for the preparation of a medicament for the treatment of a condition mediated by decreased FXR activity in a subject; the use of a compound of formula (I) for the preparation of a medicament for the treatment of obesity; the use of a compound of formula (I) for the preparation of a medicament for the treatment of diabetes mellitus in a subject; the use of a compound of formula (I) for the preparation of a medicament for the treatment of metabolic syndrome in a subject; the use of a compound of formula (I) for the preparation of a medicament for the treatment of cholestatic liver disease in a subject; the use of a compound of formula (I) for the preparation of a medicament for the treatment of organ fibrosis in a subject; and the use of a compound of formula (I) for the preparation of a medicament for the treatment of liver fibrosis in a subject.

In another aspect, the present invention provides a pharmaceutical composition comprising a compound of formula (I) for use in the treatment of a condition mediated by decreased FXR activity.

Further aspects of the present invention are described in the description of particular embodiments, examples, and claims which follow.

### DETAILED DESCRIPTION OF THE PREFERRED EMBODIMENTS

As used herein, “a compound of the invention” or “a compound of formula (I)” or “(I-A),” etc. means a compound of formula (I) (or (I-A)) or a pharmaceutically acceptable salt or solvate thereof. Similarly, with respect to isolatable intermediates such as for example, compounds of formula (II), (III), (IV), (V), (XL), (XLI) and (XLII), the phrase “a compound of formula (number)” means a compound having that formula or a pharmaceutically acceptable salt or solvate thereof.

As used herein, the term “alkyl” refers to aliphatic straight or branched saturated hydrocarbon chains containing 1-8 carbon atoms. Examples of “alkyl” groups as used herein include but are not limited to methyl, ethyl, n-propyl, isopropyl, n-butyl, isobutyl, t-butyl, pentyl, hexyl, octyl and the like.

The term “fluoroalkyl” as used herein refers to an alkyl as defined above substituted with one or more fluoro. In on particular embodiment, fluoroalkyl refers to an alkyl substituted with two or more fluoro (particularly CF₃).

The term “alkylene” refers to a straight or branched alkyl bridge, i.e., the group -alkyl-, wherein alkyl is as defined above.

As used herein, the term “halo” refers to any halogen atom. i.e., fluorine, chlorine, bromine or iodine.

As used herein, the term “alkenyl” refers to an aliphatic straight or branched unsaturated hydrocarbon chain containing 2-8 carbon atoms and at least one and up to three carbon-carbon double bonds. Examples of “alkenyl” groups as used herein include but are not limited to ethenyl and propenyl.

As used herein, the term “cycloalkyl” refers to a non-aromatic monocyclic carbocyclic ring having from 3 to 8 carbon atoms (unless a different number of atoms is specified) and no carbon-carbon double bonds. “Cycloalkyl” includes by way of example cyclopropyl, cyclobutyl, cyclopentyl, cyclohexyl, cycloheptyl and cyclooctyl. Particular cycloalkyl groups include C₃₋₆cycloalkyl.

As used herein, the term “cycloalkenyl” refers to a non-aromatic monocyclic carbocyclic ring having from 3 to 8 carbon atoms (unless a different number of atoms is specified) and from 1 to 3 carbon-carbon double bonds. “Cycloalkenyl” includes by way of example cyclopropenyl, cyclobutenyl, cyclopentenyl, cyclohexenyl, cycloheptenyl and cyclooctenyl. Particular cycloalkenyl groups include C₃₋₆cycloalkenyl.

As used herein, the term “optionally” means that the subsequently described event(s) may or may not occur, and includes both event(s) that occur and events that do not occur.

The present invention relates to a compound of formula (I):

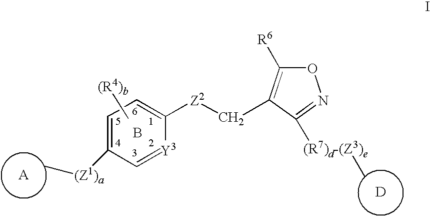

wherein:
Ring A is selected from

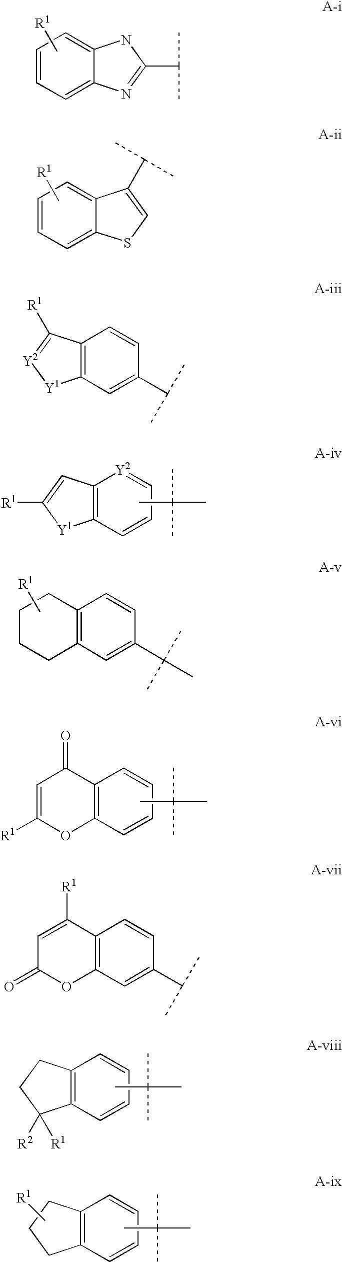

wherein
R¹ is selected from —CO₂H, —C(O)NH₂, —CO₂alkyl, and an acid equivalent group;
R² is H or —OH;
Y¹ is selected from —CH₂—, —NH—, —O— and —S—;
Y² is selected from —CH— and —N—; or
   Ring A is a substituted naphthalene when a=1;

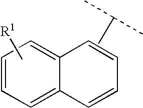

Z¹ is —NH— or —S—;
a is 0 or 1;
each R⁴ is selected from halo, alkyl and fluoroalkyl;
b is 0, 1 or 2, except that when b is 2 and Y³ is C, R⁴ is not bound at position 2 or 6 of Ring B;
Y³ is —N— or —CH—;
Z² is —O—, —S— or —N(R⁵)—, wherein R⁵ is H or alkyl;
R⁶ is selected from alkyl, 2,2,2-trifluoroethyl, C₃₋₆cycloalkyl, alkenyl, C₃₋₆cycloalkenyl and fluoro-substituted C₃₋₆cycloalkyl;
R⁷ is —C₁₋₃alkylene-;
Z³ is —O—, —S(O)_{c}—, or —NH—, where c is 0, 1 or 2;
d and e are both 0 or d is 1 and e is 0 or 1;
   Ring D is selected from C₃₋₆cycloalkyl and a moiety selected from formula D-i, D-ii and D-iii:

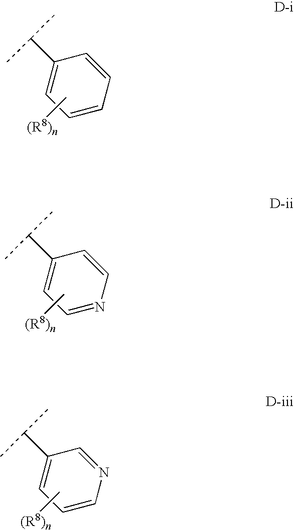

wherein
n is 0, 1, 2 or 3;
each R⁸ is the same or different and is independently selected from halo, alkyl, alkenyl, —O-alkyl, haloalkyl, —O-haloalkyl, hydroxyl substituted alkyl, and —OCF₃
   and pharmaceutically acceptable salts thereof.

In one preferred embodiment of the invention, A is A-iii:

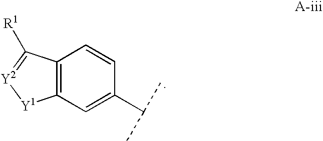

In another preferred embodiment of the invention, A is

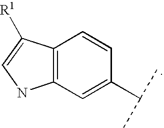

In another preferred embodiment of the invention, A is

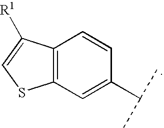

In another preferred embodiment of the invention, A is

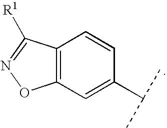

In another preferred embodiment of the invention, A is

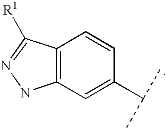

In another preferred embodiment of the invention, A is A-iv:

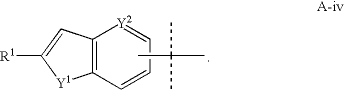

In another preferred embodiment of the invention, A is

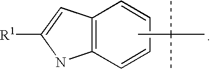

In another preferred embodiment of the invention, A is

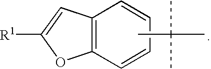

In another preferred embodiment of the invention, A is

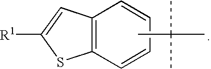

In another preferred embodiment of the invention, A is

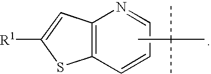

In all embodiments, R¹ is —CO₂H, —C(O)NH₂, —CO₂alkyl or acid equivalent group. In one preferred embodiment R¹ is —CO₂H or —CO₂alkyl, such as —CO₂CH₂CH₃, or any subset thereof. In another preferred embodiment, R¹ is —CO₂H.

In one embodiment of the invention, a is 1 and Z¹ is —S—. In another embodiment, a is 1 and Z¹ is —NH—.

In one embodiment of the invention, a is 0. In another embodiment, a is 1.

In one embodiment of the invention, b is 0. In the embodiment of the invention where b is 1, R⁴ is halo (particularly F or Cl), —CH₃, —CF₃, —CH₂CH₃, or any subset thereof.

In another embodiment of the invention where b is 1, R⁴ is halo.

In one embodiment of the invention, Y³ is —CH—.

In one embodiment of the invention, Z² is —O—, —S—, or —N(H)—. In one preferred embodiment, Z² is —O—.

In one embodiment, R⁶ is alkyl, 2,2,2-trifluoroethyl, C₃₋₆cycloalkyl, or any subset thereof. Specific examples of groups defining R⁶ include but are not limited to methyl, ethyl, propyl, isopropyl, t-butyl, n-butyl, isobutyl, 2,2,2-trifluoroethyl, cyclopropyl, cyclobutyl, cyclopentyl and cyclohexyl. In one embodiment, R⁶ is isopropyl, isobutyl, 2,2,2-trifluoroethyl, cyclopropyl, cyclobutyl, cyclopentyl, or any subset thereof. In one embodiment, R⁶ is isopropyl, isobutyl, cyclopropyl or cyclobutyl. In one particular embodiment, R⁶ is isopropyl or isobutyl. In one preferred embodiment, R⁶ is isopropyl.

In one particular embodiment, the invention includes compounds of formula I′ wherein d is 0 and e is 0 and thus Ring D is bound directly to the isoxazole ring as shown in formula (I′):

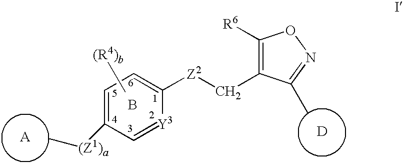

wherein all other variables are as defined above including particular and preferred embodiments thereof.

The invention includes compounds of formula (I″) wherein d is 1 and e is 0 or 1 and thus Ring D is bound to C₁₋₃ alkylene (R⁷) or Z³ (when d is 1) as shown in formula (I″).

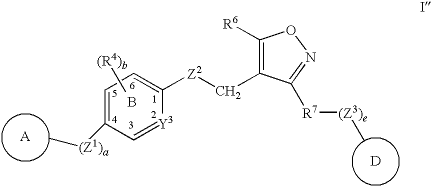

wherein all other variables are as defined above including particular and preferred embodiments thereof.

In one embodiment, wherein d is 1, R⁷ is preferably methylene or ethylene. In the embodiment wherein both d and e are 1, R⁷ is preferably methylene. In one embodiment, d is 1, e is 1 and Z³ is 0. In one particular embodiment, d is 1, e is 1, R⁷ is methylene and Z³ is O, as in formula (I′″).

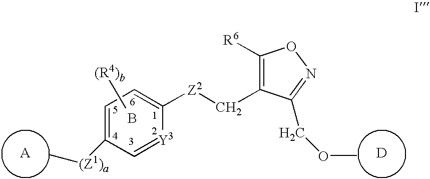

wherein all other variables are as defined above. The invention includes compounds of formula I′″.

Ring D is selected from C₃₋₆cycloalkyl and a moiety selected from formula D-i, D-ii and D-iii:

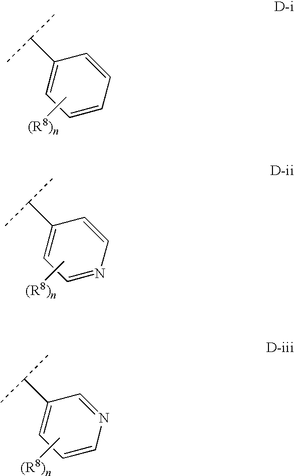

wherein
n is 0, 1, 2 or 3;
each R⁸ is the same or different and is independently selected from halo, alkyl, alkenyl, —O-alkyl, haloalkyl, hydroxyl substituted alkyl, and —OCF₃.

In one embodiment Ring D is a moiety of formula D-i.

In one embodiment wherein Ring D is a moiety of formula D-i, n is 1, 2 or 3 and each R⁸ is the same or different and is halo or alkyl. In one particular embodiment wherein Ring D is a moiety of formula D-i, n is 1, 2 or 3, R⁸ is the same and is Fl, Cl, or methyl. In one preferred embodiment wherein Ring D is a moiety of formula D-i, n is 1, 2 or 3 and R⁸ is Cl.

In one embodiment, n is 2. In one particular embodiment wherein Ring D is a moiety of formula D-i and n is 2, each R⁸ is the same or different and is halo or alkyl. In one particular embodiment wherein Ring D is a moiety of formula D-i and n is 2, each R⁸ is the same and is F, Cl, or methyl. In one preferred embodiment wherein Ring D is a moiety of formula D-i and n is 2, each R⁸ is Cl.

In one embodiment, n is 1, 2 or 3 and R⁸ is the same or different and is halo or alkyl. In another embodiment, n is 2 and R⁸ is the same or different and is halo or alkyl. In another embodiment, n is 1, 2 or 3 and R⁸ the same or different and is F, Cl, or methyl. In one preferred embodiment, n is 1, 2 or 3 and R⁸ is Cl. In another preferred embodiment, n is 2 and R⁸ is Cl.

In another embodiment, n is 2 or 3, R⁸ is the same and is Fl, Cl, or methyl. In another embodiment, n is 2 or 3, R⁸ is the same and is Cl. In a preferred embodiment, n is 2 and R⁸ is Cl.

Specific examples of particular compounds of the present invention are selected from the group consisting of:
5-[4-({[3-(2,6-Dichlorophenyl)-5-(1-methylethyl)-4-isoxazolyl]methyl}oxy)phenyl]-1H-indole-2-carboxylic acid;
6-[4-({[3-(2,6-Dichlorophenyl)-5-(1-methylethyl)-4-isoxazolyl]methyl}oxy)phenyl]-2,3-dihydro-1H-indene-1-carboxylic acid;
6-[4-({[3-(2,6-Dichlorophenyl)-5-(1-methylethyl)-4-isoxazolyl]methyl}oxy)phenyl]-1H-indole-3-carboxylic acid;
5-[4-({[3-(2,6-Dichlorophenyl)-5-(1-methylethyl)-4-isoxazolyl]methyl}oxy)phenyl]-1-benzofuran-2-carboxylic acid;
6-[4-({[3-(2,6-Dichlorophenyl)-5-(1-methylethyl)-4-isoxazolyl]methyl}oxy)phenyl]-1H-indole-2-carboxylic acid;
5-[4-({[3-(2,6-Dichlorophenyl)-5-(1-methylethyl)-4-isoxazolyl]methyl}oxy)phenyl]-2,3-dihydro-1H-indene-2-carboxylic acid;
5-[4-({[3-(2,6-Dichlorophenyl)-5-(1-methylethyl)-4-isoxazolyl]methyl}oxy)phenyl]-1-hydroxy-2,3-dihydro-1H-indene-1-carboxylic acid;
6-[4-({[3-(2,6-Dichlorophenyl)-5-(1-methylethyl)-4-isoxazolyl]methyl}oxy)phenyl]-1H-indazole-3-carboxylic acid;
3-[4-({[3-(2,6-Dichlorophenyl)-5-(1-methylethyl)-4-isoxazolyl]methyl}oxy)phenyl]-1-benzothiophene-5-carboxylic acid;
5-[4-({[3-(2,6-Dichlorophenyl)-5-(1-methylethyl)-4-isoxazolyl]methyl}oxy)phenyl]thieno[3,2-b]pyridine-2-carboxylic acid;
6-[4-({[3-(2,6-Dichlorophenyl)-5-(1-methylethyl)-4-isoxazolyl]methyl}oxy)phenyl]-1-benzothiophene-3-carboxylic acid;
5-[4-({[3-(2,6-Dichlorophenyl)-5-(1-methylethyl)-4-isoxazolyl]methyl}oxy)phenyl]-1-benzothiophene-2-carboxylic acid;
6-[6-({[3-(2,6-Dichlorophenyl)-5-(1-methylethyl)-4-isoxazolyl]methyl}oxy)-3-pyridinyl]-1H-indole-3-carboxylic acid;
6-[4-({[3-(2,6-Dichlorophenyl)-5-(1-methylethyl)-4-isoxazolyl]methyl}oxy)phenyl]-4-oxo-4H-chromene-2-carboxylic acid;
7-[4-({[3-(2,6-Dichlorophenyl)-5-(1-methylethyl)-4-isoxazolyl]methyl}oxy)phenyl]-2-oxo-2H-chromene-4-carboxylic acid;
7-[4-({[3-(2,6-Dichlorophenyl)-5-(1-methylethyl)-4-isoxazolyl]methyl}oxy)phenyl]-4-oxo-4H-chromene-2-carboxylic acid;
6-[4-({[3-(2,6-Dichlorophenyl)-5-(1-methylethyl)-4-isoxazolyl]methyl}oxy)phenyl]-1,2,3,4-tetrahydro-1-naphthalene carboxylic acid;
8-{[4-({[3-(2,6-Dichlorophenyl)-5-(1-methylethyl)-4-isoxazolyl]methyl}oxy)phenyl]amino}-2-naphthalene carboxylic acid;
4-{[4-({[3-(2,6-Dichlorophenyl)-5-(1-methylethyl)-4-isoxazolyl]methyl}oxy)phenyl]thio}-1-benzothiophene-2-carboxylic acid;
Ethyl 6-[4-({[3-(2,6-dichlorophenyl)-5-(1-methylethyl)-4-isoxazolyl]methyl}oxy)phenyl]-1,2-benzisoxazole-3-carboxylate;
2-[4-({[3-(2,6-Dichlorophenyl)-5-(1-methylethyl)-4-isoxazolyl]methyl}oxy)phenyl]-1H-benzimidazole-4-carboxylic acid;
   and pharmaceutically acceptable salts thereof.

One preferred compound of the invention is 6-[4-({[3-(2,6-dichlorophenyl)-5-(1-methylethyl)-4-isoxazolyl]methyl}oxy)phenyl]-1H-indole-3-carboxylic acid; or a pharmaceutically acceptable salt thereof. In one particular embodiment, 6-[4-({[3-(2,6-dichlorophenyl)-5-(1-methylethyl)-4-isoxazolyl]methyl}oxy)phenyl]-1H-indole-3-carboxylic acid; or pharmaceutically acceptable salt thereof is in crystalline form. In one preferred embodiment, the compound of the invention is 6-[4-({[3-(2,6-dichlorophenyl)-5-(1-methylethyl)-4-isoxazolyl]methyl}oxy)phenyl]-1H-indole-3-carboxylic acid (i.e. the form of the acid).

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centers are inverted.

Suitable pharmaceutically acceptable salts according to the present invention will be readily determined by one skilled in the art and will include, for example, salts prepared from inorganic bases such as lithium hydroxide, sodium hydroxide, potassium hydroxide, lithium hydride, sodium hydride, potassium hydride, lithium carbonate, lithium hydrogen carbonate, sodium carbonate, sodium hydrogen carbonate, potassium carbonate, potassium hydrogen carbonate, as well as potassium tert-butoxide and organic bases such as diethyl amine, lysine, arginine, choline, tris (hydroxymethyl)aminomethane (tromethamine), triethanolamine, diethanolamine, and ethanolamine.

When used in medicine, the salts of a compound of formula (I) should be pharmaceutically acceptable, but pharmaceutically unacceptable salts may conveniently be used to prepare the corresponding free base or pharmaceutically acceptable salts thereof.

As used herein, the term “solvate” refers to a crystal form containing the compound of formula (I) or a pharmaceutically acceptable salt thereof and either a stoichiometric or a non-stoichiometric amount of a solvent. Solvents, by way of example, include water (thus producing hydrates), methanol, ethanol, or acetic acid. Hereinafter, reference to a compound of formula (I) is to any physical form of that compound, unless a particular form, salt or solvate thereof is specified.

Processes for preparing pharmaceutically acceptable salts of the compounds of formula (I) are conventional in the art. See, e.g., Burger's Medicinal Chemistry And Drug Discovery 5th Edition, Vol 1: Principles And Practice.

As will be apparent to those skilled in the art, in the processes described below for the preparation of compounds of formula (I), certain intermediates, may be in the form of pharmaceutically acceptable salts of the compound. Those terms as applied to any intermediate employed in the process of preparing compounds of formula (I) have the same meanings as noted above with respect to compounds of formula (I). Processes for preparing pharmaceutically acceptable salts of such intermediates are known in the art and are analogous to the process for preparing pharmaceutically acceptable salts of the compounds of formula (I).

In one embodiment, the compounds of formula (I) are FXR agonists. As used herein, the term “FXR agonist” refers to compounds which exhibit a pEC₅₀ greater than 4 in the FXR Cofactor Recruitment Assay described below. More particularly, FXR agonists are compounds which exhibit a pEC₅₀ greater than 5 in the FXR Cofactor Recruitment Assay described below.

Compounds of formula (I) are useful in therapy in subjects such as mammals, and particularly humans. In particular, the compounds of formula (I) are useful in the treatment of a condition mediated by decreased FXR activity in a subject such as a mammal, particularly a human. As used herein, the term “treatment” includes the prevention of occurrence of symptoms of the condition or disease in the subject, the prevention of recurrence of symptoms of the condition or disease in the subject, the delay of recurrence of symptoms of the condition or disease in the subject, the decrease in severity or frequency of outward symptoms of the condition or disease in the subject, slowing or eliminating the progression of the condition and the partial or total elimination of symptoms of the disease or condition in the subject.

Conditions which have been reported to be mediated by a decreased FXR activity include but are not limited to dyslipidemia (Sinal, C., et al. 2000 Cell 102:731-744; Zhang, Y., et al., 2006 Proc. Nat. Acad. Sci., U.S.A., 103:1006-1011); cardiovascular diseases such as atherosclerosis (Hanniman, E. A., et al., J. Lipid Res. 2005, 46:2595-2604); obesity (Chen, L., et al., 2006 Diabetes 55 suppl. 1:A200; Cariou, B., et al., 2006 J. Biol. Chem. 281:11039-11049; Rizzo, G., et al. 2006 Mol. Pharmacol. 70:1164-1173); diabetes mellitus (Duran-Sandoval, D., et al., 2004 Diabetes 53:890-898; Bilz, S., et al., 2006 Am. J. Physiol. Endocrinol. Metab. 290:E716-E722; Nozawa, H., 2005 Biochem. Biophys. Res. Commun. 336:754-761; Duran-Sandoval, D., et al., 2005 Biochimie 87:93-98; Claudel, T., et al., 2005 Arterioscler. Thromb. Vasc. Biol. 25:2020-2030; Duran-Sandoval, D., et al., 2005 J. Biol. Chem. 280:29971-29979; Savkur, R. S., et al., 2005 Biochem. Biophys. Res. Commun., 329:391-396; Cariou, B., et al., 2006 J. Biol. Chem. 281:11039-11049; Ma, K., et al., 2006 J. Clin. Invest. 116:1102-1109; Zhang, Y., et al., 2006 Proc. Nat. Acad. Sci. U.S.A. 103:1006-1011); metabolic syndrome (Chen, L., et al., 2006 Diabetes 55 suppl. 1:A200); disorders of the liver such as cholestatic liver disease (Liu, Y. et al., 2003 J. Clin. Invest. 112:1678-1687) and cholesterol gallstone disease (Moschetta, A., et al., 2004 Nat. Med. 10:1352-1358); organ fibrosis (Fiorucci, S., et al. 2004 Gastroenterology 127:1497-1512 and Fiorucci, S., et al., 2005 J. Pharmacol. Exp. Ther. 314:584-595) including liver fibrosis (Fiorucci, S., et al. 2004 Gastroenterology 127:1497-1512); inflammatory bowel disease (Inagaki, T., et al., 2006 Proc. Nat. Acad. Sci., U.S.A. 103:3920-3925); and liver regeneration (Huang, W., et al., 2006 Science 312:233-236).

Compounds of formula (I) are believed to be useful for the treatment of dyslipidemia in a subject, such as a mammal, particularly a human. The compounds of the present invention are currently believed to increase the flow of bile acid. Increased flow of bile acids improves the flux of bile acids from the liver to the intestine. FXR null mice demonstrate that FXR not only plays a role in bile acid homeostasis, but also plays a role in lipid homeostasis by virtue of the regulation of enzymes and transporters that are involved in lipid catabolism and excretion.

Compounds of formula (I) are also believed to be useful for lowering triglycerides in a subject, such as a mammal, particularly a human. As used herein “lowering triglycerides” means lowering triglycerides in a subject in need thereof below the initial level of triglyercides in that subject before administration of a compound of formula (I). For example, the compounds of formula (I) may lower triglycerides by decreasing fat absorption, decreasing hepatic triglyceride production or decreasing hepatic triglyceride secretion. The compounds of formula (I) may also lower serum and hepatic triglycerides.

By treating dyslipidemia, compounds of formula (I) are currently believed to be useful in the treatment of hypertriglyceridemia and hypercholesteronemia related cardiovascular disease such as atherosclerosis in a subject such as a mammal, particularly a human. Compounds of formula (I) are also believed to be useful for the treatment of non-alcoholic fatty liver disease and non-alcoholic steatohepatitis in a subject, such as a mammal, particularly a human (Chen, L., et al., 2006 Diabetes 55 suppl. 1:A200; Watanabe, M., et al., 2004 J. Clin. Invest., 113:1408-1418).

The compounds of formula (I) are useful for the treatment of obesity in a subject, such as a mammal, particularly a human.

Compounds of formula (I) are also useful for the treatment of diabetes mellitus in a subject, such as a mammal, particularly a human. For example, the compounds of formula (I) are useful for the treatment of type 2 diabetes. The effects of an FXR agonist, GW4064, on body weight, glucose tolerance, serum glucose, serum insulin, serum triglyceride, and liver triglyceride contents via oral administration have been observed in an high-fat diet induced insulin resistant, glucose intolerant, and obese mouse model (Chen, L., et al., 2006 Diabetes 55 suppl. 1:A200). Male 20 to 25 g C57BL mice (Charles River, Indianapolis, Ind.) were housed at 72° F. and 50% relative humidity with a 12 h light and dark cycle and fed with standard rodent chow (Purina 5001, Harlan Teklad, Indianapolis, Ind.) or a high-fat diet (TD93075, Harlan Teklad, Indianapolis, Ind.) for seven weeks. After two weeks, mice on high-fat diet were randomized to vehicle or treatment groups. There were no significant difference in body weight, body fat mass, serum glucose and insulin, and area under the curve (AUC) for glucose in glucose tolerance test (GTT) between the vehicle group and the treatment group. Starting from the fourth week, mice were given either vehicle or GW4064 (100 mg/kg) twice a day orally. Mice on the standard rodent chow were also given vehicle as a control. At the end of the third week of compound treatment, a GTT was performed and body composition was measured using the quantitative magnetic resonance (QMR) method. At the end of the study (fourth week of compound treatment), blood samples were taken from inferior vena cava and tissue samples were collected for further analysis. Blood glucose during GTT was measured using Bayer Glucometer Elite® XL. Serum chemistry levels were measured using the Instrumentation Laboratory Ilab600™ clinical chemistry analyzer (Instrumentation Laboratory, Boston, Mass.). Liver triglyceride contents were measured using the methanolic-KOH saponification method and a triglyceride assay kit (GPO-TRINDER, Sigma Diagnostics, St. Louis, Mo.). The results indicated that GW4064 reduced the high-fat diet induced body weight gain. It is believed that the result may have been due to a decrease in fat mass. GW4064 also appeared to improve glucose tolerance, decreased serum glucose, insulin and triglyceride, and reduced liver triglyceride content. In addition, Cariou and colleagues treated male ob/ob mice with GW4064 (30 mg/kg) intraperitoneally (2006 J. Biol. Chem. 281:11039-11049). GW4064 treatment did not alter body weight as well as food intake. Whereas GW4064 had no effect on fasting blood glucose in ob/ob mice, it decreased insulin concentration in the treated group. GW4064 treated ob/ob mice also showed an improved glucose tolerance and enhanced insulin sensitivity compared to controls. In another study, it was reported that GW4064 significantly improved hyperglycemia and hyperlipidemia in diabetic db/db mice (Zhang, Y., et al, 2006 Proc. Nat. Acad. Sci. U.S.A. 103:1006-1011). Oral GW4064 (30 mg/kg, bid) treatment decreased blood glucose, serum β-hydroxybutyrate, triglyceride, NEFA, and total cholesterol in db/db mice. It was also demonstrated that GW4064 treatment enhanced insulin signalling and glycogen storage in the liver of db/db mice. These data suggest that FXR agonists, including the compounds of the formula (I), may be used for the treatment of obesity, insulin resistance, glucose intolerance, diabetes mellitus, fatty liver disease and metabolic syndrome.

Compounds of formula (I) are also useful for the treatment of metabolic syndrome in a subject, such as a mammal, particularly a human. Metabolic syndrome is characterized by a group of metabolic risk factors in one person. They include abdominal obesity (excessive fat tissue in and around the abdomen), atherogenic dyslipidemia (high triglycerides, low high density lipoprotein (HDL) cholesterol and high low density lipoprotein (LDL) cholesterol), elevated blood pressure, insulin resistance or glucose intolerance, prothrombotic state and proinflammatory state. People with metabolic syndrome are at increased risk of coronary heart disease and atherosclerosis-related diseases (e.g., stroke and peripheral vascular disease) and type 2 diabetes mellitus. There are several clinical criteria for metabolic syndromes including ATP III, WHO, and AACE (American Association of Clinical Endocrinologists) (see tables, for review see Grundy, S. M., et al., 2004 Circulation 109:433-438). The present invention provides a method for the treatment of metabolic syndrome characterized by abdominal obesity, atherogenic dyslipidemia and insulin resistance with or without glucose interance, and may benefit other components of metabolic syndrome in a subject.

TABLE 1
ATP III Clinical Identification of the Metabolic Syndrome

| Risk Factor | Defining Level |
|---|---|
| Abdominal obesity, given as | |
| waist circumference*^{†} | |
| Men | >102 cm (>40 in) |
| Women | >88 cm (>35 in) |
| Triglycerides | ≧150 mg/dL |
| HDL cholesterol | |
| Men | <40 mg/dL |
| Women | <50 mg/dL |
| Blood pressure | ≧130/≧85 mmHg |
| Fasting glucose | ≧110 mg/dt^{‡} |

*Overweight and obesity are associated with insulin resistance and the metabolic syndrome. However, the presence of abdominal obesity is more highly correlated with the metabolic risk factors than is an elevated BMI. Therefore, the simple measure of waist circumference is recommended to identify the body weight component of the metabolic syndrome.
^{†}Some male patients can develop multiple metabolic risk factors when the waist circumference is only marginally increased, eg, 94 to 102 cm (37 to 39 in). Such patients may have a strong genetic contribution to insulin resistance. They should benefit from changes in life habits, similarly to men with categorical increases in waist circumference.
^{‡}The American Diabetes Association has recently established a cutpoint of ≧100 mg/dL, above which persons have either prediabetes (impaired fasting glucose) or diabetes. This new cutpoint should be applicable for identifying the lower boundary to define an elevated glucose as one criterion for the metabolic syndrome.

TABLE 2
WHO Clinical Criteria for Metabolic Syndrome

Insulin resistance, identified by 1 of the following:
Type 2 diabetes
Impaired fasting glucose
Impaired glucose tolerance
or for those with normal fasting glucose levels (<110 mg/dL), glucose
uptake below the lowest quartile for background population under
investigation under hyperinsulinemic, euglycemic conditions
Plus any 2 of the following:
Antihypertensive medication and/or high blood pressure (≧140 mmHg
systolic or ≧90 mmHg diastolic)
Plasma triglycerides ≧150 mg/dL (≧1.7 mmol/L)
HDL cholesterol <35 mg/dL (<0.9 mmol/L) in men or <39 mg/dL
(1.0 mmol/L) in women
BMI >30 kg/m² and/or waist:hip ratio >0.9 in men, >0.85 in women
Urinary albumin excretion rate ≧2.0 μg/min or albumin:creatinine
ratio ≧30 mg/g

TABLE 3
AACE Clinical Criteria for Diagnosis of the Insulin Resistance
Syndrome*

| Risk Factor Components | Cutpoints for Abnormality |
|---|---|
| Overweight/obesity | BMI ≧25 kg/m² |
| Elevated triglycerides | ≧150 mg/dL (1.69 mmol/L) |
| Low HDL cholesterol | |
| Men | <40 mg/dL (1.04 mmol/L) |
| Women | <50 mg/dL (1.29 mmol/L) |
| Elevated blood pressure | ≧130/85 mmHg |
| 2-Hour postglucose | >140 mg/dL |
| challenge | |
| Fasting glucose | Between 110 and 126 mg/dL |
| Other risk factors | Family history of type 2 diabetes, |
| | hypertension, or CVD |
| | Polycystic ovary syndrome |
| | Sedentary lifestyle |
| | Advancing age |
| | Ethnic groups having high risk for type |
| | 2 diabetes or CVD |

*Diagnosis depends on clinical judgment based on risk factors.

Compounds of formula (I) are believed to be useful for the treatment of cholestatic liver disease. For example, the compounds of formula (I) are believed to be useful in the treatment of primary biliary cirrhosis or primary sclerosing cholangitis. FXR therefore is a target for the treatment of a number of cholestatic liver diseases and non-alcoholic steatohepatitis. The compounds of formula (I) are also believed to be useful for the treatment of gall stones. For example, the compounds of formula (I) are believed to be useful in the treatment of cholesterol gallstone disease. The compounds of formula (I) are also believed to be useful for decreasing liver lipid accumulation.

Compounds of formula (I) are also believed to be useful for the treatment of organ fibrosis. Fibrotic disorders can be characterized as acute or chronic, but share the common characteristic of excessive collagen accumulation and an associated loss of function as normal tissues are replaced or displaced by fibrotic tissues. Acute forms of fibrosis include response to trauma, infections, surgery, burns, radiation and chemotherapy. Chronic forms of fibrosis may be due to viral infection, diabetes mellitus, obesity, fatty liver, hypertension, scleroderma and other chronic conditions that induce fibrosis.

Organs that are most commonly affected by fibrosis include liver, kidney, and lung. Organ fibrosis can cause the progressive loss of organ function. Retroperitoneal fibrosis (including idiopathic retroperitoneal fibrosis) may not originate from any major organ, but can involve and adversely affect the function of organs such as the kidneys.

Accordingly, as used herein, the term fibrosis refers to all recognized fibrotic disorders, including fibrosis due to pathological conditions or diseases, fibrosis due to physical trauma (‘traumatic fibrosis’), fibrosis due to radiation damage, and fibrosis due to exposure to chemotherapeutics. As used herein, the term “organ fibrosis” includes but is not limited to liver fibrosis, fibrosis of the kidneys, fibrosis of lung, and fibrosis of the intestine. “Traumatic fibrosis” includes but is not limited to fibrosis secondary to surgery (surgical scarring), accidental physical trauma, burns, and hypertrophic scarring.

In one embodiment, compounds of formula (I) are useful for the treatment of liver fibrosis in a subject, particularly a mammal, such as a human, in need of treatment thereof. As used herein, “liver fibrosis” includes liver fibrosis due to any cause, including but not limited to virally-induced liver fibrosis such as that due to hepatitis B or C virus; exposure to alcohol (alcoholic liver disease), certain pharmaceutical compounds including but not limited to methotrexate, some chemotherapeutic agents, and chronic ingestion of arsenicals or vitamin A in megadoses, oxidative stress, cancer radiation therapy or certain industrial chemicals including but not limited to carbon tetrachloride and dimethylnitrosamine; and diseases such as primary biliary cirrhosis, primary sclerosing colangitis, fatty liver, obesity, non-alcoholic steatohepatitis, cystic fibrosis, hemochromatosis, auto-immune hepatitis, and steatohepatitis. Current therapy in liver fibrosis is primarily directed at removing the causal agent, e.g., removing excess iron (e.g., in the case of hemochromatosis), decreasing viral load (e.g., in the case of chronic viral hepatitis), or eliminating or decreasing exposure to toxins (e.g., in the case of alcoholic liver disease). Anti-inflammatory drugs such as corticosteroids and colchicine are also known for use in treating inflammation that can lead to liver fibrosis. Other strategies for treating liver fibrosis are under development (see, e.g., Murphy, F., et al., 2002 Expert Opin. Invest. Drugs 11:1575-1585; Bataller, R. and Brenner, D. A., 2001 Sem. Liver Dis. 21:437-451). Thus in another embodiment, the present invention provides a method for the treatment of liver fibrosis in a subject which comprises administering a therapeutically effective amount of a compound of formula (I) in combination with another therapeutic agent useful for the treatment of symptoms associated with liver fibrosis. Examples of therapeutic agents useful for the treatment of symptoms associated with liver fibrosis include corticosteroids and cholchicine.

The response of the liver to hepatocellular damage, similar to wound healing in other tissues, includes inflammation and tissue remodeling, with associated changes in the quantity and quality of the extracellular matrix. Progressive accumulation of extracellular matrix proteins, including collagen types I and III, eventually distorts the architecture of the liver by forming fibrous scars, resulting in disrupted blood flow and an eventual deterioration in hepatic function. (Bissell, D. M. and Maher, J. J., “Hepatic Fibrosis and Cirrhosis.” Ed. Zakim, D. and Thomas, D. B., 4 ed. 2 vols. Philadelphia: Saunders, 2003. 395-416, Hanauske-Abel, H. M., “Fibrosis of the Liver: Representative Molecular Elements and Their Emerging Role As Anti-Fibrotic Targets.” Ed. Zakim, D., and Thomas, D. B., 4 ed. 2 vols. Philadelphia: Saunders, 2003. 347-394). Hepatic stellate cells (HSC) have been identified as important mediators of the fibrotic process in the liver, and are believed to be primarily responsible for the synthesis of excess extracellular matrix seen in liver disease. Liver injury can result in quiescent HSCs converting to activated myofibroblast-like cells that proliferate, migrate, recruit inflammatory cells, and synthesize collagens and other extracellular matrix proteins. (Bissell, D. M. and Maher, J. J., “Hepatic Fibrosis and Cirrhosis.” Ed. Zakim, D. and Thomas, D. B., 4 ed. 2 vols. Philadelphia: Saunders, 2003. 395-416, Hanauske-Abel, H. M., “Fibrosis of the Liver: Representative Molecular Elements and Their Emerging Role As Anti-Fibrotic Targets.” Ed. Zakim, D., and Thomas, D. B., 4 ed. 2 vols. Philadelphia: Saunders, 2003. 347-394). Various cytokines are reported to activate HSCs, including transforming growth factor β (TGFβ). Following liver injury, HSCs synthesize α-smooth muscle actin (α-SMA) as part of the migration response, consequently a marked accumulation of α-SMA can be seen at areas of active liver fibrogenesis. (Bissell, D. M. and Maher, J. J., “Hepatic Fibrosis and Cirrhosis.” Ed. Zakim, D. and Thomas, D. B., 4 ed. 2 vols. Philadelphia: Saunders, 2003. 395-416, Hanauske-Abel, H. M., “Fibrosis of the Liver: Representative Molecular Elements and Their Emerging Role As Anti-Fibrotic Targets.” Ed. Zakim, D., and Thomas, D. B., 4 ed. 2 vols. Philadelphia: Saunders, 2003. 347-394). Derangement of the normal epithelial/mesenchymal interaction, characterised by cholangiocyte damage/proliferation, can also lead to extracellular matrix-producing and progressive fibrogenesis. (Pinzani, M., et al., 2004 Digest. Liver Dis. 36:231-242.)

As is known in the art, liver fibrosis may be clinically classified into five stages of severity (S0 to S4), usually based on histological examination of a biopsy specimen. S0 indicates no fibrosis, whereas S4 indicates cirrhosis. While various criteria for staging the severity of liver fibrosis exist, in general early stages of fibrosis are identified by discrete, localized areas of scarring in one portal (zone) of the liver, whereas later stages of fibrosis are identified by bridging fibrosis (scarring that crosses zones of the liver).

Compounds of formula (I) are also useful for the treatment of inflammatory bowel disease in a subject, such as a mammal, particularly a human. Inflammatory bowel disease (IBD) is defined as a group of idiopathic relapsing inflammatory disorders of the bowel—the large or small intestine. The pathogenesis of IBD remains Obscure and may involve genetic, environmental and immunological factors. (Drossman, D. A. 1999 Aliment Pharmacol. Ther. 13(s2):3-14; Danese, S., et al. 2004 Autoimmunity Reviews 3: 394-400; Stokkers, P. C. F. and Hommes, D. W. 2004 Cytokine 28:167-173.) The most common types of inflammatory bowel disease are ulcerative colitis and Crohn disease.

Compounds of formula (I) are also believed to be useful for enhancing liver regeneration in a subject, such as a mammal, particularly a human. For example, the compounds of formula (I) are believed to be useful for enhancing liver regeneration for liver transplantation.

The present invention provides a method for the treatment of a condition mediated by decreased FXR activity, particularly a condition in which a FXR agonist may be useful, in a subject, such as a mammal, particularly a human, in need thereof. The present invention also provides the use of a compound of formula (I) for the preparation of a medicament for the treatment of a condition mediated by decreased FXR activity, particularly a condition in which a FXR agonist may be useful, in a subject, such as a mammal, particularly a human in need thereof.

The present invention also provides a method for lowering triglycerides in a subject, such as a mammal, particularly a human, in need thereof. The present invention also provides the use of a compound of formula (I) for the preparation of a medicament for lowering triglycerides in a subject. In one embodiment, the compound of formula (I) is 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof. In another embodiment, the compound of formula (I) is 5-[4-({[3-(2,6-dichlorophenyl)-5-(1-methylethyl)-4-isoxazolyl]methyl}oxy)phenyl]-1H-indole-2-carboxylic acid or a pharmaceutically acceptable salt thereof.

The present invention provides a method for the treatment of obesity in a subject, such as a mammal, particularly a human, in need thereof. The present invention also provides the use of a compound of formula (I) for the preparation of a medicament for the treatment of obesity in a subject. In one embodiment, the compound of formula (I) is 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof. In another embodiment, the compound of formula (I) is 5-[4-({[3-(2,6-dichlorophenyl)-5-(1-methylethyl)-4-isoxazolyl]methyl}oxy)phenyl]-1H-indole-2-carboxylic acid or a pharmaceutically acceptable salt thereof.

The present invention provides a method for the treatment of diabetes mellitus in a subject, such as a mammal, particularly a human, in need thereof. The present invention also provides the use of a compound of formula (I) for the preparation of a medicament for the treatment of diabetes mellitus in a subject. In one embodiment, the compound of formula (I) is 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof. In another embodiment, the compound of formula (I) is 5-[4-({[3-(2,6-dichlorophenyl)-5-(1-methylethyl)-4-isoxazolyl]methyl}oxy)phenyl]-1H-indole-2-carboxylic acid or a pharmaceutically acceptable salt thereof.

The present invention provides a method for the treatment of metabolic syndrome in a subject, such as a mammal, particularly a human, in need thereof. The present invention also provides the use of a compound of formula (I) for the preparation of a medicament for the treatment of metabolic syndrome in a subject. In one embodiment, the compound of formula (I) is 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof. In another embodiment, the compound of formula (I) is 5-[4-({[3-(2,6-dichlorophenyl)-5-(1-methylethyl)-4-isoxazolyl]methyl}oxy)phenyl]-1H-indole-2-carboxylic acid or a pharmaceutically acceptable salt thereof.

The present invention provides a method for the treatment of cholestatic liver disease in a subject, such as a mammal, particularly a human, in need thereof. The present invention also provides the use of a compound of formula (I) for the preparation of a medicament for the treatment of cholestatic liver disease in a subject. In one embodiment, the compound of formula (I) is 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof. In another embodiment, the compound of formula (I) is 5-[4-({[3-(2,6-dichlorophenyl)-5-(1-methylethyl)-4-isoxazolyl]methyl}oxy)phenyl]-1H-indole-2-carboxylic acid or a pharmaceutically acceptable salt thereof.

The present invention provides a method for the treatment of organ fibrosis in a subject, such as a mammal, particularly a human, in need thereof. The present invention also provides the use of a compound of formula (I) for the preparation of a medicament for the treatment of organ fibrosis in a subject. In one embodiment, the compound of formula (I) is 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof. In another embodiment, the compound of formula (I) is 5-[4-({[3-(2,6-dichlorophenyl)-5-(1-methylethyl)-4-isoxazolyl]methyl}oxy)phenyl]-1H-indole-2-carboxylic acid or a pharmaceutically acceptable salt thereof.

The present invention provides a method for the treatment of liver fibrosis in a subject, such as a mammal, particularly a human, in need thereof. The present invention also provides the use of a compound of formula (I) for the preparation of a medicament for the treatment of liver fibrosis in a subject. In one embodiment, the compound of formula (I) is 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof. In another embodiment, the compound of formula (I) is 5-[4-({[3-(2,6-dichlorophenyl)-5-(1-methylethyl)-4-isoxazolyl]methyl}oxy)phenyl]-1H-indole-2-carboxylic acid or a pharmaceutically acceptable salt thereof.

All of the methods of the present invention comprise the step of administering a therapeutically effective amount of the compound of formula (I). As used herein, the term “therapeutically effective amount” refers to an amount of a compound of formula (I) which is sufficient to achieve the stated effect in the subject to which it is administered. Accordingly, a therapeutically effective amount of a compound of formula (I) used in the method for the treatment of a condition mediated by decreased FXR activity in a human will be an amount sufficient for the treatment of the condition mediated by decreased FXR activity in a human. A therapeutically effective amount of a compound of formula (I) for use in the method for the treatment of diabetes mellitus in a human will be an amount sufficient for the treatment of diabetes mellitus in a human. A therapeutically effective amount of a compound of formula (I) for use in the method for the treatment of metabolic syndrome in a human will be an amount sufficient for the treatment of metabolic syndrome in a human. A therapeutically effective amount of a compound of formula (I) for use in the method for the treatment of organ (e.g., liver) fibrosis in a human will be an amount sufficient for the treatment of organ fibrosis in a human.

The amount of a compound of formula (I) which is required to achieve the desired therapeutic or biological effect will depend on a number of factors such as the use for which it is intended, the means of administration, the recipient and the type and severity of the condition or disease being treated, and will be ultimately at the discretion of the attendant physician or veterinarian. In general, a typical daily dose for the treatment of a disease or condition mediated by decreased FXR activity in a human, for instance, may be expected to lie in the range of from about 0.01 mg/kg to about 100 mg/kg for a 70 kg human. This dose may be administered as a single unit dose or as several separate unit doses or as a continuous infusion. Similar dosages would be applicable for the treatment of other diseases, conditions and therapies including diabetes mellitus and obesity in humans.

While it is possible that, for use in therapy, a therapeutically effective amount of a compound of formula (I) may be administered as the raw chemical, it is typically presented as the active ingredient of a pharmaceutical composition or formulation. Accordingly, the invention further provides a pharmaceutical composition comprising a compound of the formula (I). The pharmaceutical composition may further comprise one or more pharmaceutically acceptable carriers or diluents. The carrier(s) and/or diluent(s) must be acceptable in the sense of being compatible with the other ingredients of the formulation and not deleterious to the recipient thereof. In one particular embodiment, the compound is in crystalline form. In accordance with another aspect of the invention there is also provided a process for the preparation of a pharmaceutical formulation including admixing a compound of the formula (I) with one or more pharmaceutically acceptable carriers and/or diluents.

Pharmaceutical formulations may be presented in unit dose form containing a predetermined amount of active ingredient per unit dose. Such a unit may contain a therapeutically effective dose of the compound of formula (I) or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethyl-cellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of active ingredient. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

A compound of formula (I) can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

A compound of formula (I) may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in 1986 Pharmaceutical Research 3:318.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Pharmaceutical compositions adapted for nasal administration wherein the carrier is a solid include a coarse powder having a particle size for example in the range of about 20 microns to about 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered, dose pressurised aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

In the above-described methods of treatment and uses, a compound of formula (I) may be employed alone, in combination with one or more other compounds of formula (I) or in combination with other therapeutic agents. Thus, the present invention also encompasses pharmaceutical compositions further comprising one or more therapeutic agents. In one embodiment, the pharmaceutical compositions further comprise one or more lipid-altering agents. Examples of lipid-altering agents include but are not limited to liver X receptor (LXR) agonists described in PCT Publication No. WO02/24632 to GlaxoSmithKline. Examples of other therapeutic agents include, but are not limited to, 3-Hydroxy-3-Methyl-Glutaryl-CoA reductase inhibitors such as statins (atorvastatin, fluvastatin, pravastatin, lovastatin, cerivastatin, and nisvastatin); squalene epoxidase inhibitors, squalene synthetase inhibitors, bile acid transport inhibitors (BATi), human peroxisome proliferator activated receptor (PPAR) gamma agonists such as rosiglitazone, troglitazone, and pioglitazone and thiazolidinediones; PPAR α agonists such as clofibrate, fenofibrate and gemfibronzil; PPAR dual α/γ agonists; cyclooxygenase-2 (COX-2) inhibitors such as rofecoxib and celecoxib; thrombin inhibitors; acyl-coenzyme A; cholesterol acyltransferase (ACAT) inhibitors including selective ACAT inhibitors; microsomal triglyceride transfer protein (MTP) inhibitors; probucol, niacin; cholesterol absorption inhibitors; bile acid sequestrants; LDL receptor inducers; platelet aggregation inhibitors such as glycoprotein IIb/IIIa fibrinogen receptor antagonists and aspirin; vitamin B6 and pharmaceutically acceptable salts thereof; vitamin B12; folic acid or a pharmaceutically acceptable salt or ester thereof; antioxidant vitamins such as C and E and beta carotene; beta blockers; angiotensin II antagonists such as losartan; antiotensin converting enzyme inhibitors such as enalapril and captopril; calcium channel blockers such as nifedipine and diltiazam; endothelian antagonists; agents other than LXR ligands that enhance ATP-Binding Cassette Transporter-A1 gene expression; and bisphosphonate compounds such as alendronate sodium.

The methods and uses employing these combinations may comprise the administration of the compound of formula (I) and another therapeutic agent either sequentially in any order or simultaneously in separate or combined pharmaceutical compositions. When combined in the same composition it will be appreciated that the compounds must be stable and compatible with each other and the other components of the composition and may be formulated for administration. When formulated separately they may be provided in any convenient formulation, in such a manner as are known for such compounds in the art.

When a compound of formula (I) is used in combination with another therapeutic agent, the dose of each compound may differ from that when the compound is used alone. Appropriate doses will be readily appreciated by those skilled in the art. The appropriate dose of the compound(s) of formula (I) and the other therapeutically active agent(s) and the relative timings of administration will be selected in order to achieve the desired combined therapeutic effect, and are within the expertise and discretion of the attendant clinician.

Compounds of the invention can be made according to any suitable method of organic chemistry. As will be apparent to those skilled in the art and as depicted in the schemes which follow, the order of the steps in each reaction is not critical to the practice of the processes of the present invention. The reaction steps depicted in each scheme may be carried out in any suitable order based upon the knowledge of those skilled in the art.

Further, it will be apparent to those skilled in the art that certain reaction steps may be most efficiently performed by installing protecting groups prior to the reaction, which are removed subsequently. The choice of protecting groups as well as general techniques for their installation and removal are within the skill of those in the art. It will be appreciated by those skilled in the art that certain ring systems represented in the generic ring structure of the A ring will require the use of a protective group to minimize the possibility of undesired side reactions from occurring. The protective group may be easily installed by methods contained in the literature and likewise may be removed once they are no longer needed. Examples of ring systems that would require a protective group would include benzimidazole, indazole and indole.

According to one method, a compound of formula (I) may be prepared using the process depicted in Scheme 1, below.

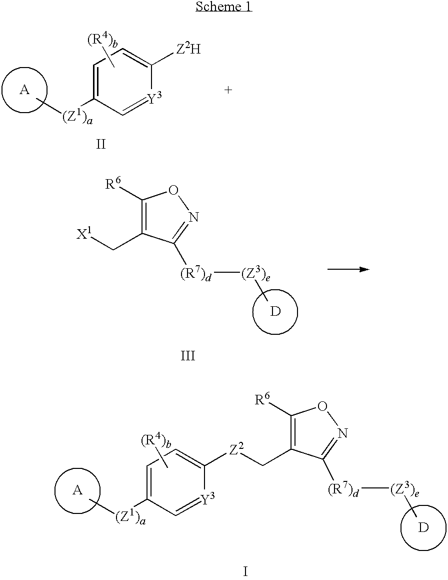

wherein X¹ is chloride, iodide, bromide, triflate, tosylate, nosylate, besylate or mesylate, (preferably chloro);
R¹ is —CO₂alkyl;
if A is A-viii, then R² is H; and
all other variables are as defined above for formula (I).

In general, the process for preparing a compound of formula (I) as depicted in Scheme 1 comprises the steps of:

a) reacting a compound of formula (II) with a compound of formula (III) to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

A compound of formula (I), prepared by any suitable process, may be converted into a pharmaceutically acceptable salt thereof or may be converted to a different compound of formula (I) or a pharmaceutically acceptable salt or solvate thereof using techniques described herein below and those conventional in the art.

More particularly, the compound of formula (I) may be prepared by reacting the compound of formula (II) with a compound of formula (III) in the presence of a suitable base such as cesium carbonate or potassium carbonate, in a polar aprotic solvent, such as N,N-dimethylformamide, at ambient or elevated temperature.

The compound of formula (III) may be prepared by reacting a compound of formula (IV) with the appropriate reagent to prepare a compound having the desired leaving group (X¹).

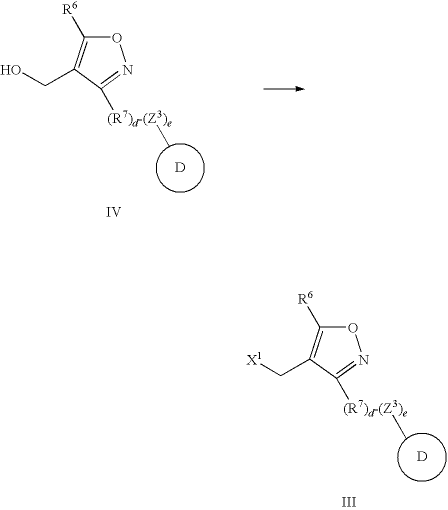

wherein X¹ is chloride, iodide, bromide, triflate, tosylate, nosylate, besylate or mesylate, (preferably chloro); and
all other variables are as defined above.

In the embodiment wherein X¹ is halide, the reaction is performed by halogenating the compound of formula (IV). Any suitable halogenating reagent conventional in the art may be employed in the reaction. Examples of suitable halogenating reagents include, but are not limited to, thionyl chloride and triphenylphosphine dichloride.

The reaction is typically carried out in a non-polar solvent such as dichloromethane or 1,2-dichloroethane at ambient temperature.

In the embodiment wherein X¹ is triflate, tosylate or meslyate, the reaction may be carried out according to the conventional methods. See, Vedejs, E., et al., 1977 J. Org. Chem. 42:3109-3113; Handy, S. T., et al. 2004 J. Org. Chem. 69:2362-2366; and Copp, F. C., et al. 1955 J. Chem. Soc. 2021-2027.

The compound of formula (IV) may be prepared by reducing a compound of formula (V).

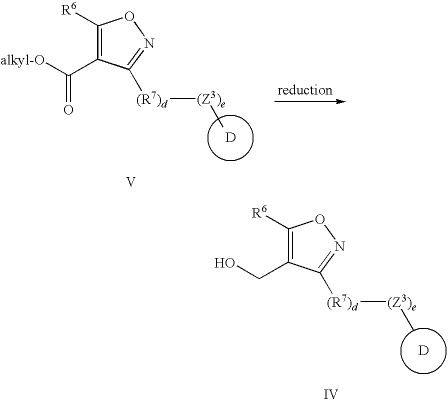

wherein all variables are as defined above.

A compound of formula (V) may be treated with a reducing agent, such as diisobutylaluminum hydride, in a suitable solvent, such as tetrahydrofuran.

In another embodiment, the compound of formula (V) may be saponified to the corresponding carboxylic acid prior to reducing with a suitable reducing agent, such as borane, to prepare a compound of formula (IV). In addition, the carboxylic acid may also be converted to a mixed anhydride before reducing with a reducing agent such as sodium borohydride to prepare a compound of formula (IV).

Compounds of formula (V) may be prepared by multiple routes. In one embodiment, the compound of formula (V) may be prepared by a process comprising the steps of:

a) chlorinating a compound of formula (VI); and
b) cyclizing with a β-ketoester of formula (VII).

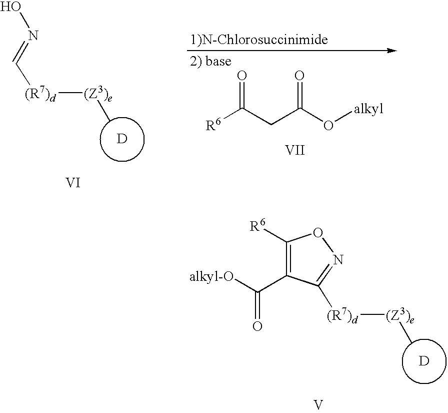

wherein all variables are as defined above.

The process is conveniently carried out according to the method described by Doyle, F. P., et. al., 1963 J. Chem. Soc. 5838-5845. Esters of formula (VII) are commercially available or can be prepared using conventional techniques.

The compound of formula (VI) may be prepared by condensing a compound of formula (VIII) with hydroxylamine.

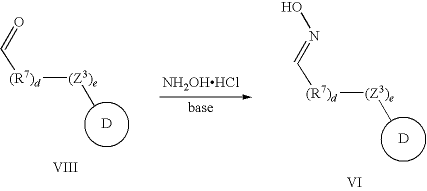

wherein all variables are as defined above.

Conditions suitable for this condensation reaction are conventional in the art.

Compounds of formula (VIII) are commercially available or can be prepared using conventional techniques.

In another embodiment, a compound of formula (V) may be prepared by a process comprising the steps of: a) reacting a compound of formula (IX) with tin chloride in the presence of a compound of formula (VII) to prepare a compound of formula (X) and b) reacting the compound of formula (X) with hydroxylamine to yield a compound of formula (V). See, Singh, B. and Lesher, G. Y. 1978 Synthesis 829-830. The compound of formula (V) may then be reduced with a suitable reducing agent, such as diisobutylaluminum hydride, in the manner described above, to prepare a compound of formula (IV).

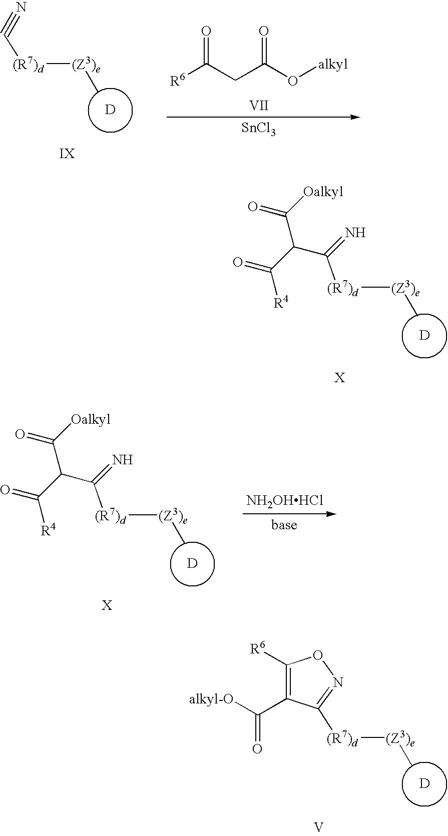

wherein all variables are as defined above.

The compound of formula (IX) may be obtained commercially or prepared by procedures in the literature. See, Guo, H. and Zhang, Y. 2000 Syn. Commun. 30:1879-1885.

A compound of formula (II) may be prepared by coupling the compound of formula (XI) with a boronic acid or ester compound of formula (XII) using conventional Suzuki coupling techniques.

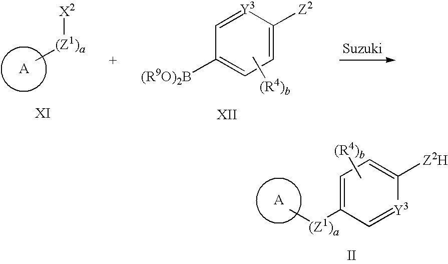

wherein: X² is chloro, bromo, iodo, or triflate;
a is 0;
Y³ is —CH—;
R¹ is —CO₂alkyl;
if A is A-viii, then R² is H;
R⁹ is alkyl or H; and
all other variables are as defined above.

For example, the compound of formula (II) may be prepared by coupling a compound of formula (XI) with a compound of formula (XII) in the presence of a suitable palladium complex such as tetrakis(triphenylphosphine)palladium(0) and a base such as sodium carbonate in a mixture of water and ethereal solvent such as 1,2-dimethoxyethane, at an elevated temperature. In another example, the compound of formula (II) may be prepared by coupling a compound of formula (XI) with a compound of formula (XII) in the presence of palladium(II)acetate and triphenylphosphine using a base such as potassium phosphate in water and a solvent such as dioxane, at an elevated temperature.

A compound of formula (XII) and formula (XI) may be synthesized by techniques known to those skilled in the art or may be purchased commercially.

For example, in one embodiment, a compound of formula (XI-a) in which X² is triflate may be synthesized from a phenol of formula (XIII). Reagents suitable for installing the triflate include but are not limited to triflic anhydride. The reaction may be carried out in a solvent, such as dichloromethane and in the presence of a suitable base, such as pyridine or triethylamine.

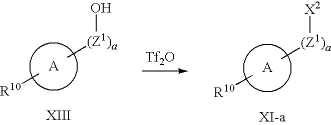

wherein X² is triflate;
Tf₂O is trifluoromethylsulfonic anhydride;
a is 0;
R¹⁰ is CO₂tBu; and
all other variables are as defined above.

In another embodiment, a compound of formula (XI-a) may be prepared by reacting the compound of formula (XIII) in a suspension of toluene with an aqueous solution of tribasic potassium phosphate and then reacting with triflic anhydride.

A compound of formula (XIII) may be synthesized by techniques known to those skilled in the art or may be purchased commercially.

For example, in one embodiment, a compound of formula of (XIII-a) may be synthesized by reacting a compound of formula (XIV) with hydrogen using a catalytic amount of palladium on carbon in a solvent system like methanol and chloroform.

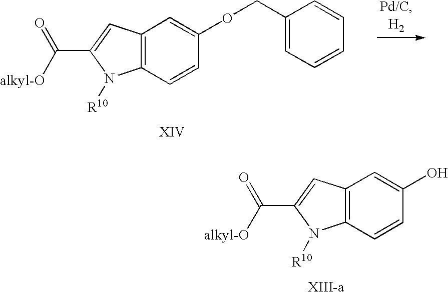

wherein R¹⁰ is CO₂tBu; and
all other variables are as defined above.

A compound of formula (XIV) may be synthesized by reacting a compound of formula (XV) with bis(1,1-dimethylethyl)dicarbonate and a catalytic amount of N,N-dimethylaminopyridine in a solvent like tetrahydrofuran at room temperature.

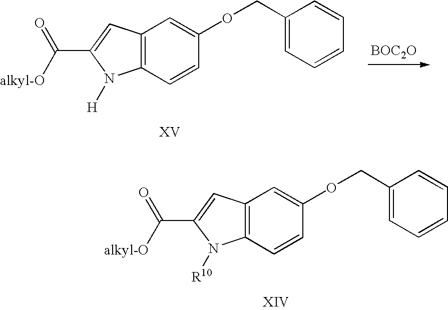

wherein BOC₂O is bis(1,1-dimethylethyl)dicarbonate; R¹⁰ is CO₂tBu; and
all other variables are as defined above.

A compound of formula (XV) may be synthesized by techniques known to those skilled in the art or may be purchased commercially.

In another embodiment, a compound of formula (XIII-b) may be synthesized by the steps of:

1) reacting a compound of formula (XVIII) with boron tribromide in a solvent, such as dichloromethane, followed by aqueous workup with sodium bicarbonate;
2) optionally the resulting mixture may then be reesterified by heating with an alcoholic solvent, such as methanol, and thionyl chloride or an acid catalyst, such as sulfuric acid, to form a compound of formula (XIII-b).

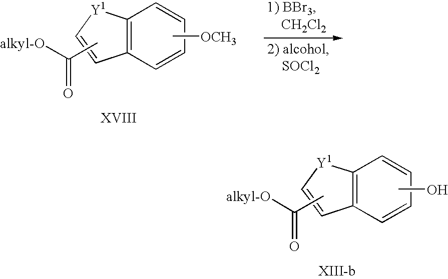

wherein Y¹ is —O—, —S— or —NH—.

As an example, a compound of formula (XVIII-a) may be prepared by reacting a compound of formula (XIX) with a mixture of trichloroacetyl chloride and aluminum chloride in a chlorinated solvent like dichloromethane at reduced temperature. The resulting trichloride intermediate may be reacted with aqueous potassium hydroxide to yield a compound of formula (XVIII-a).

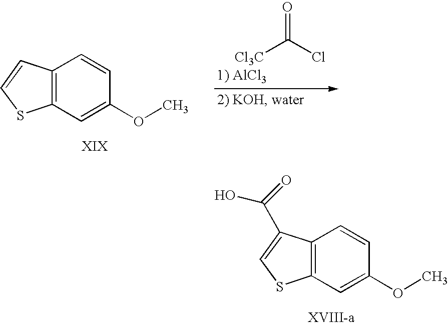

A compound of formula (XIX) may be prepared by reacting of a compound of formula (XX) with a solution of boron trifluoride diethyletherate in a chlorinated solvent like dichloromethane.

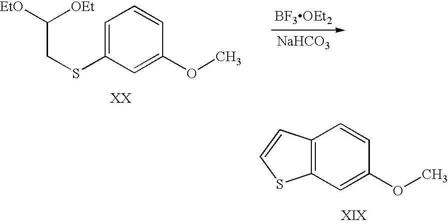

wherein Et=ethyl.

A compound of formula (XX) may be prepared by reacting of a compound of formula (XXI) with bromoacetaldehyde diethyl acetal and a base like potassium carbonate in solvent like acetone.

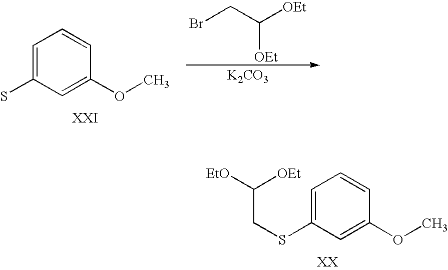

wherein Et=ethyl.

Compound (XXI) may be made by methods known to those skilled in the art or may be purchased commercially.

In an additional embodiment, a compound of formula (XIII-c) may be prepared by first reacting a compound of formula (XL) with hydrobromic acid in acetic acid. The mixture is then concentrated and then reacted with an alcohol and an acid catalyst or an agent which can generate an acid catalyst, such as thionyl chloride.

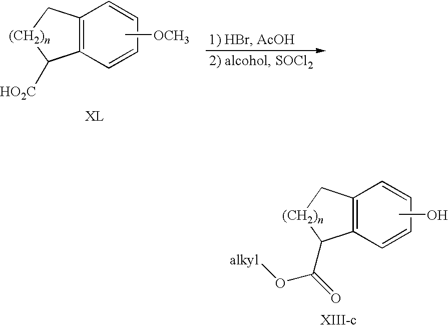

wherein n is 1 or 2.

A compound of formula (XL) may be purchased from commercial sources or may be synthesized by methods described in the literature.

In another embodiment, a compound of formula (XI-b) in which X² is chloro or bromo may be synthesized by reacting a compound of formula (XVI) with bis(1,1-dimethylethyl)dicarbonate and a catalytic amount of N,N-dimethylaminopyridine in a solvent such as tetrahydrofuran at room temperature.

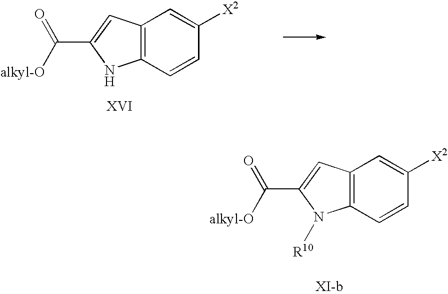

wherein X² is chloro or bromo;
R¹⁰ is CO₂tBu; and
all other variables are as defined above.

A compound of formula (XVI) in which X² is chloro or bromo may be synthesized by techniques known to those skilled in the art or may be purchased commercially.

A compound of formula (XI-c) may be synthesized by the steps of:

1) hydrogenating a compound of formula (XVII) using a suitable metallic catalyst like palladium on carbon;
2) converting the resulting phenol to the triflate by treatment with trifluoromethanesulfonic anhydride and a base such as triethylamine in a solvent like dichloromethane; and
3) reacting the indole nitrogen with bis(1,1-dimethylethyl)-dicarbonate to form a compound of formula (XI-c).

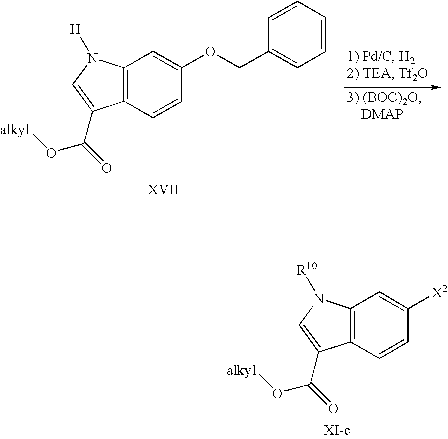

wherein X² is triflate;
TEA=triethylamine; Tf₂O trifluoromethanesulfonic anhydride;
(BOC)₂O=bis(1,1-dimethylethyl)-dicarbonate;
DMAP=N,N-dimethylaminopyridine;
R¹⁰ is CO₂tBu; and
all other variables are as defined above.

A compound of formula (XI-f) may be prepared by brominating a compound of formula (XXII) with bromine in acetic acid to yield an intermediate bromide-carboxylic acid. The intermediate can then be esterified by heating in an alcoholic solvent like methanol and thionyl chloride or an acid catalyst, such as sulfuric acid.

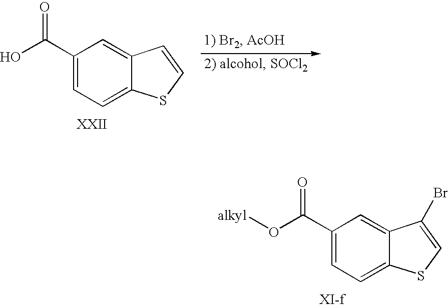

wherein AcOH is acetic acid.

A compound of formula (XXII) can be made by those skilled in the art or may be purchased commercially.

A compound of formula (XI-g) may be prepared by reacting a compound of formula (XXIX) with N-bromosuccinimide and benzoylperoxide in a solvent like carbon tetrachloride to yield an intermediate tribromide. The tribromide may then be reacted with a mixture of a dialkylmalonate and sodium hydride in a solvent like tetrahydrofuran.

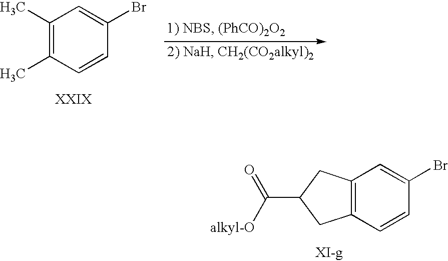

A compound of formula (XXIX) may be purchased from commercial sources or may be made using procedures in the literature.

As another example of processes for preparing a compound of formula (II), a compound of formula (II-b) may be prepared by reacting a compound of (formula (XXIII) with boron trichloride to remove the benzyl ether, then reacting the indazole with bis(1,1-dimethylethyl) dicarbonate, a base like triethylamine and a catalytic amount of dimethylaminopyridine in a solvent like dichloromethane.

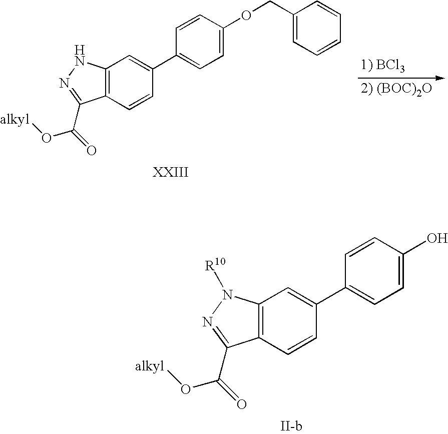

wherein (BOC)₂O=bis(1,1-dimethylethyl)-dicarbonate.

A compound of formula (XXIII) may be prepared by reacting a compound of formula (XXIV) with 1,1-dimethylethyl nitrite in a solvent like acetic acid at elevated temperatures.

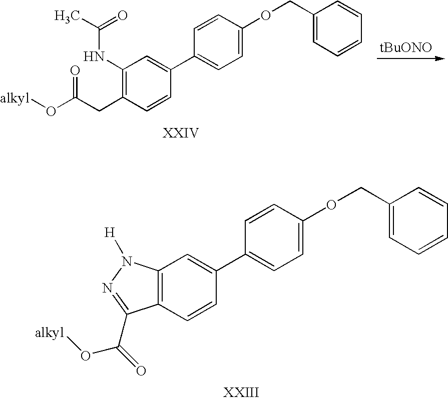

wherein tBuONO=tert-butyl nitrite.

A compound of formula (XXIV) may be prepared by reducing a compound of formula (XXV) with hydrogen using a metallic catalyst like Palladium on carbon in acetic anhydride and acetic acid to yield a phenol which can be reacted with benzyl bromide and a base like potassium carbonate in a solvent like N,N-dimethylformamide.

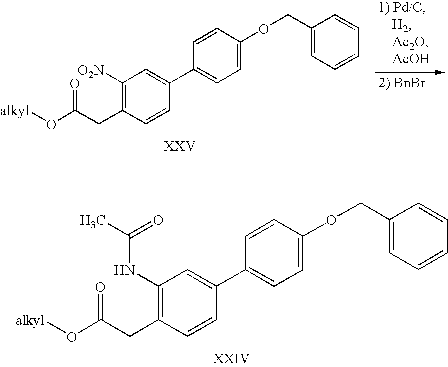

wherein Ac₂O=acetic anhydride; AcOH=acetic acid; and BnBr=benzylbromide.

A compound of formula (XXV) may be synthesized by reacting a compound of formula (XLVIII) with a boronic acid of formula (XLIX) under standard Suzuki coupling conditions. A compound of formula (XLVIII) may be prepared according to literature procedures. A compound of formula (XLIX) may be prepared by literature procedures or may be purchased from commercial sources.

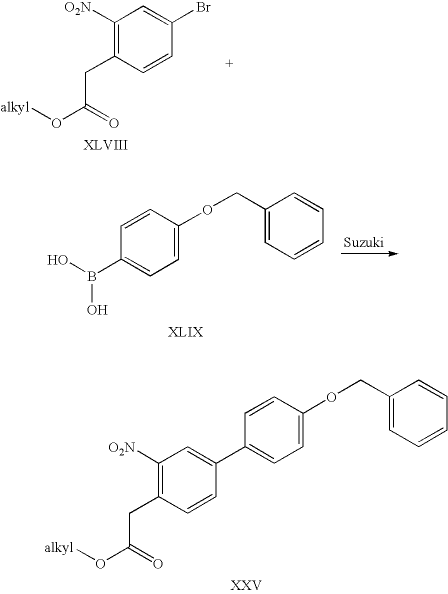

As another example of processes for preparing a compound of formula (II), a compound of formula (II-c) may be made by reacting a compound of formula (XXVI) with boron tribromide in a solvent like dichloromethane at reduced temperature.

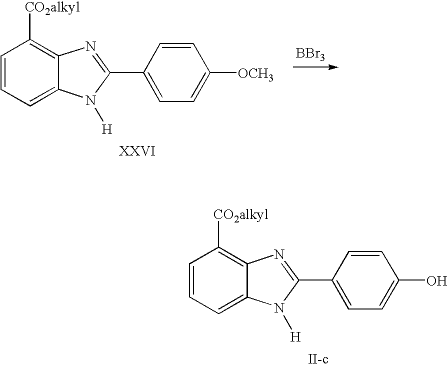

A compound of formula (XXVI) may be prepared by reacting a compound of formula (XXVII) with a mixture of triphenyl phosphine oxide, trifluoromethanesulfonic anyhydride and 4-(methyloxy)benzoic acid in a solvent like 1,2-dichloromethane at reduced temperature.

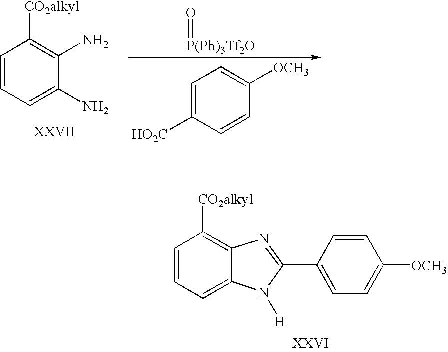

wherein P(O)(Ph)₃=triphenlyphosphine oxide; and Tf₂O=trifluoromethanesulfonic anhydride;

A compound of formula (XXVII) may be prepared by reducing a compound of formula (XXVIII) with hydrogen using a catalyst like palladium on carbon in a solvent like ethanol.

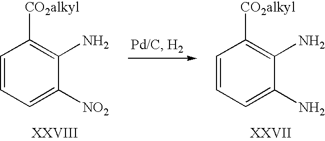

A compound of formula (XXVIII) may be purchased from commercial sources or may be made using procedures in the literature.

As another example of processes for preparing compounds of formula (II), a compound of formula (II-d) may be prepared by reacting a compound of formula (XXX) with boron trichloride in a solvent like dichloromethane.

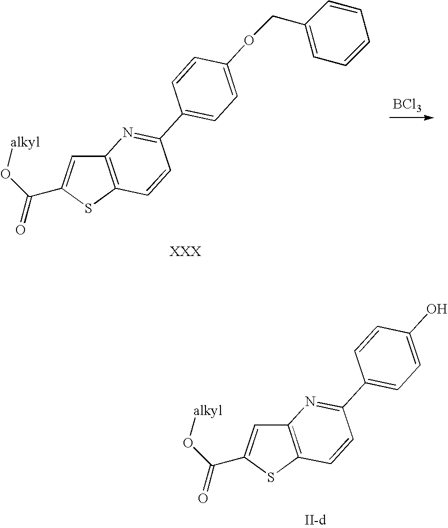

A compound of formula (XXX) may be prepared by reacting a compound of formula (XXXI) under standard Suzuki reaction conditions with {4-[(phenylmethyl)oxy]phenyl}boronic acid to yield an intermediate thienopyridine which can be deprotonated with n-butyl lithium. The resulting anion is quenched with an alkylchloroformate to yield a compound of formula (XXX).

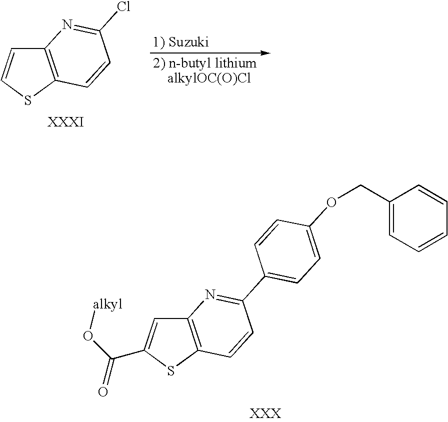

wherein alkylOC(O)Cl is an alkylchloroformate.

A compound of formula (XXXI) may be prepared by reacting a compound of formula (XXXII) with polyphosphoric acid to form an intermediate acetamide. The acetamide is then added to a mixture of phosphorus oxychloride and N,N-dimethylformamide to afford a compound of formula (XXXI).

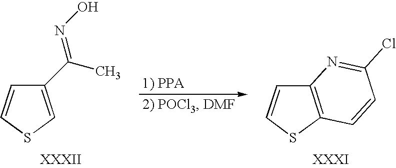

wherein PPA=polyphosphoric acid and DMF=N,N-dimethylformamide.

A compound of formula (XXXII) may be prepared by reacting a compound of formula (XOCH) with a mixture of hydroxylamine hydrochloride and a base, such as sodium acetate in ethanol, at elevated temperature.

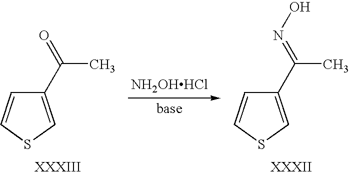

A compound of formula (XOH) may be purchased from commercial sources or may be made using procedures in the literature.

As another example of processes for preparing compounds of formula (II), a compound of formula (II-e) may be prepared by reacting a compound of formula (XXXIV) with a solution of boron tribromide in a solvent like dichloromethane at reduced temperature, for example about 0° C. The resulting product may optionally be refluxed in an alcohol with an acid catalyst to reesterify any material that may have been hydrolyzed in the previous reaction to maximize the yield of the compound of formula (II-e).

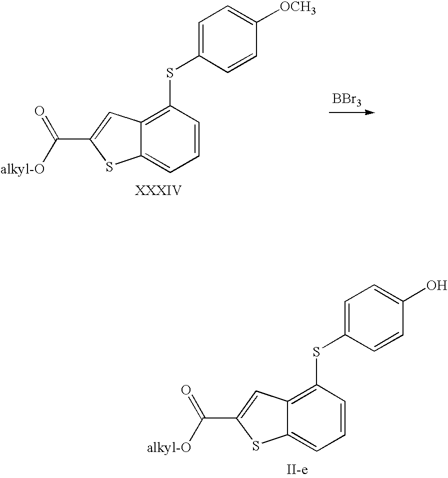

A compound of formula (XXXIV) may be made by reacting a compound of formula (XXXV) with a mixture of alkyl thioglycolate and a sodium alkoxide in N,N-dimethylformamide.

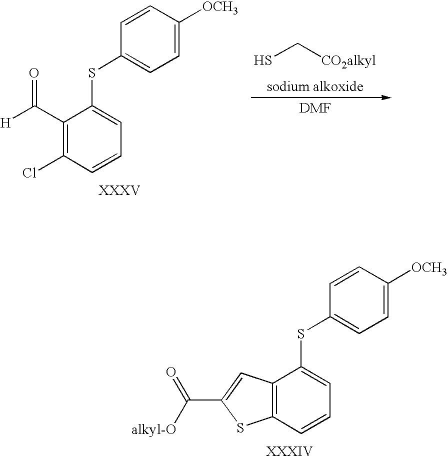

wherein DMF is N,N-dimethylformamide.

A compound of formula (XXXV) may be prepared by reacting a compound of formula (XXXVI) with a mixture of a base like potassium carbonate in N,N-dimethylformamide and a compound of formula (XXXVII).

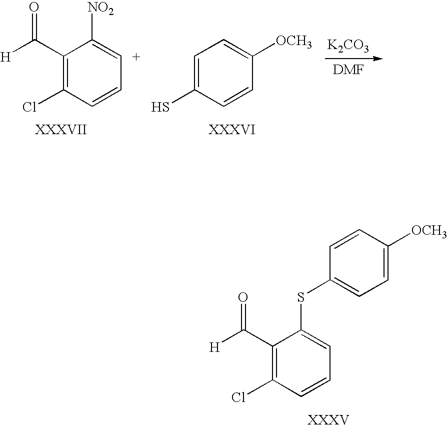

wherein DMF is N,N-dimethylformamide.

Compounds of formula (XXXVII) and (XXXVI) may be purchased from commercial sources or may be made using procedures in the literature.

As another example of processes for preparing compounds of formula (II), a compound of formula (II-f) may be prepared by reacting a compound of formula (XXXVIII) with a solution of boron tribromide in a solvent like dichloromethane at reduced temperature, for example about 0° C.

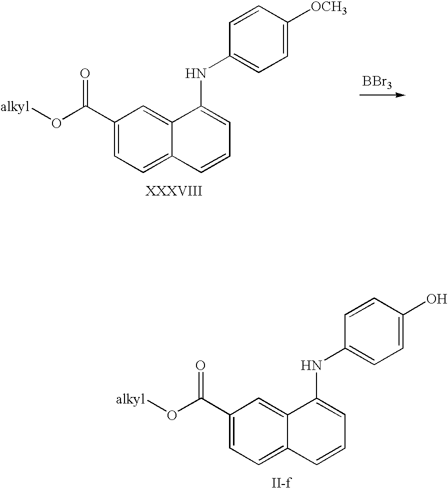

A compound of formula (XXXVIII) may be made by reacting a compound of formula (XXXVIII) with a mixture of p-anisidine, cesium carbonate and a palladium catalyst like tris(dibenzylideneacetone)dipalladium(0) and 2,2′-bis(diphenylphosphino)-1,1′-binaphthyl in a solvent like toluene at elevated temperatures.

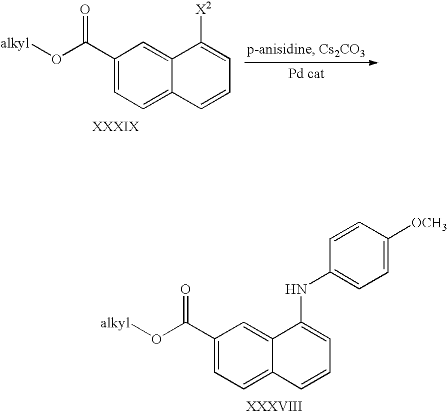

wherein X² is bromo, iodo, or triflate.

A compound of formula (XXXIX) may be purchased from commercial sources or may be made using procedures in the literature.

According to another embodiment, a compound of formula (I) may be prepared using the process depicted in Scheme 2, below.

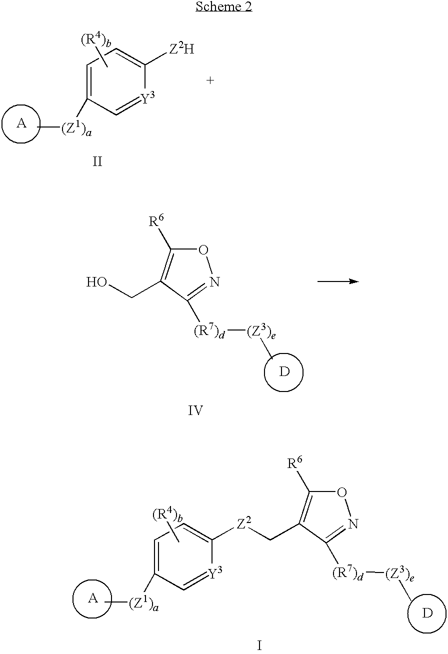

wherein
R¹ is CO₂alkyl;
if A is A-viii, then R² is H; and
all other variables are as defined above for formula (I).

In general, the process for preparing a compound of formula (I) as depicted in Scheme 2 comprises the steps of:

a) reacting a compound of formula (II) with a compound of formula (IV) to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

More specifically, the compound of formula (I) may be prepared by reacting the compound of formula (IV) with a compound of formula (II) under Mitsunobu reaction conditions. For example, a compound of formula (I) can be prepared by the reacting a compound of formula (II) with an alcohol of formula (IV) in a solution of dichloromethane or toluene with triphenyl phosphine and a dialkyl azodicarboxylate, such as diisopropyl azodicarboxylate or di-tent-butyl azodicarboxylate at an elevated temperature.

According to another embodiment, a compound of formula (I) may be prepared using the process depicted in Scheme 3, below.

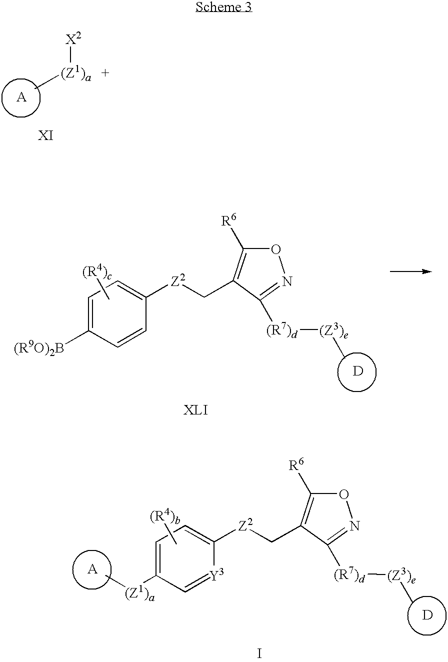

wherein:
R¹ is —CO₂alkyl;
if A is A-viii, then R² is H;
a is 0;
X² is chloro, bromo, iodo, or triflate
R⁹ is H or alkyl; and
all other variables are as defined above for formula (I).

In general, the process of Scheme 3 comprises the steps of:

a) reacting a compound of formula (XI) with a boronic acid or ester compound of formula (XLI) under Suzuki coupling conditions to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

More specifically, a compound of formula (I) may be prepared reacting a compound of formula (IX) with a compound of formula (XLI) under conventional Suzuki coupling reaction conditions as described in Scheme 1 above. A compound of formula (XI) may be prepared as described above.

A compound of formula (XLI) may be prepared by reacting a compound of formula (XLII) with a compound of formula (III) in the presence of a base such as cesium carbonate or potassium carbonate. The reaction may be carried out in a polar aprotic solvent, such as N,N-dimethylformamide.

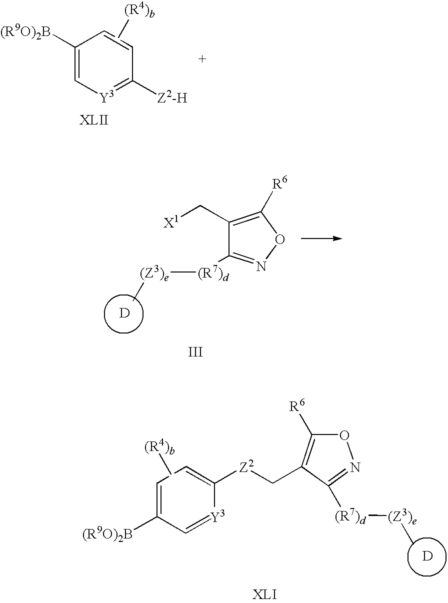

wherein:
X¹ is chloro, iodo, bromo, triflate, tosylate, nosylate, besylate or mesylate, (preferably chloro);
Y³ is CH;
R⁹ is alkyl; and
all other variables are as defined above.

The boronic ester of formula (XLI) wherein R⁹ is alkyl, may optionally be hydrolyzed to the corresponding boronic acid if desired.

A compound of formula (XLII) may be synthesized by techniques known to those skilled in the art or may be purchased commercially. A compound of formula (III) may be prepared as described above.

According to another embodiment, a compound of formula (I) may be prepared using the process depicted in Scheme 4, below.

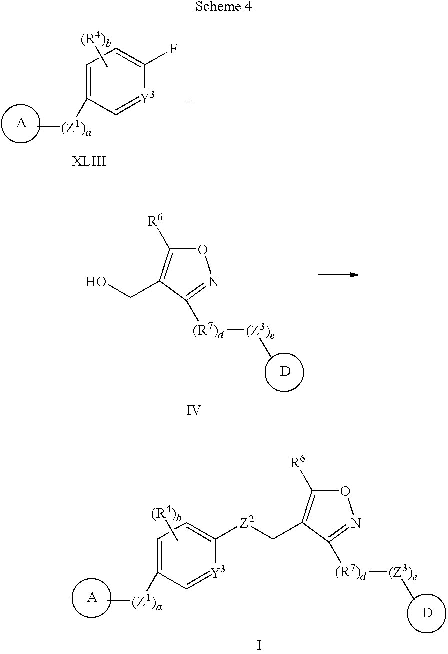

wherein R¹ is CO₂alkyl;
if A is A-viii, then R² is H;
Y³ is N;
Z² is —O—; and
all other variables are as defined above for formula (I).

In general, the process for preparing a compound of formula (I) as depicted in Scheme 4 comprises the steps of:

a) reacting a compound of formula (IV) with a base to prepare an anion;
b) condensing the anion with a compound of formula (XLIII) to prepare a compound of formula (I);
c) optionally converting the compound of formula (I) into a pharmaceutically acceptable salt thereof; and

d) optionally converting the compound of formula (I) or a pharmaceutically acceptable salt thereof into a different compound of formula (I) or a pharmaceutically acceptable salt thereof.

More specifically, the compound of formula (I) is prepared by reacting the compound of formula (IV) in a solution of 2-methyl-2-propanol with a base like potassium t-butoxide and condensing the anion formed with compound of formula (XLIII) at an elevated temperature. Conditions suitable for this condensation reaction are conventional in the art.

A compound of formula (XLIII) may be prepared by coupling the compound of formula (XI) with a boronic acid or ester compound of formula (XLIV) using conventional Suzuki coupling techniques. For example, the compound of formula (XLIII) may be prepared by coupling a compound of formula (XI) with a compound of formula (XLIV) in the presence of a suitable palladium complex such as tetrakis(triphenylphosphine)-palladium(0) and a base such as sodium carbonate in a mixture of water and ethereal solvent such as 1,2-dimethoxyethane, at an elevated temperature.

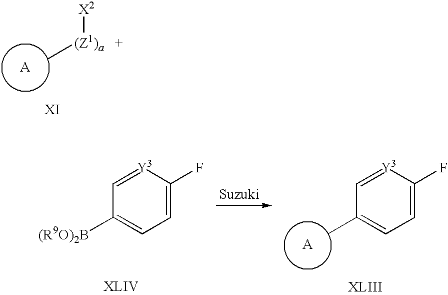

wherein: X² is chloro, bromo, iodo, or triflate;
R¹ is CO₂alkyl; if A is A-viii, then R² is H;
a is 0
Y³ is N;
R⁹ is H or alkyl; and
all other variables are as defined above.

Compounds of formula (XI) may be synthesized as previously detailed or purchased from commercial sources. Compounds of formula (XLIV) can be purchased from commercial sources.

According to another embodiment, a compound of formula (I) may be prepared using the process depicted in Scheme 5, below.

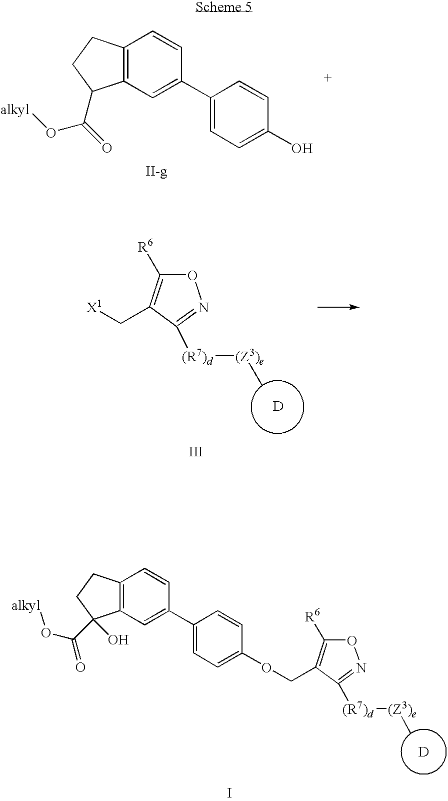

wherein X¹ is chloride; and
all other variables are as defined above for formula (I).

In general, the process for preparing a compound of formula (I) as depicted in Scheme 1 comprises the steps of:

a) reacting a compound of formula (II-g) with a compound of formula (III) to prepare a compound of formula (I);
b) optionally converting the compound of formula (I) into a pharmaceutically acceptable salt thereof; and
c) optionally converting the compound of formula (I) or a pharmaceutically acceptable salt thereof into a different compound of formula (I) or a pharmaceutically acceptable salt thereof.

More particularly, the compound of formula (I) may be prepared by reacting the compound of formula (II-g) with a compound of formula (III) in the presence of a suitable base such as cesium carbonate or potassium carbonate, in a polar aprotic solvent, such as N,N-dimethylformamide, at ambient or elevated temperature.

A compound of formula (II-g) may be prepared from a compound of formula (III-c) as described above.

A compound of formula (XIII-c) may be synthesized by reacting a compound of formula (XLV) with an alcohol and an acid catalyst at an elevated temperature.

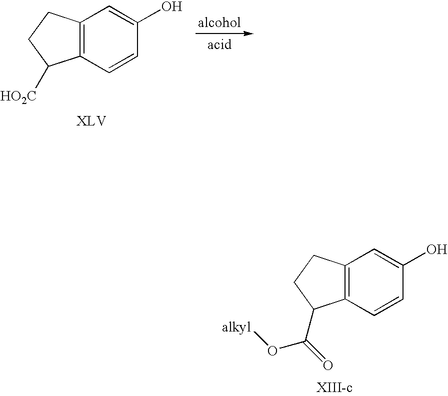

A compound of formula (XLV) may be synthesized by reacting a compound of formula (XLVI) with hydrochloric acid and acetic acid at elevated temperatures followed by reaction with hydrobromic acid at elevated temperatures.

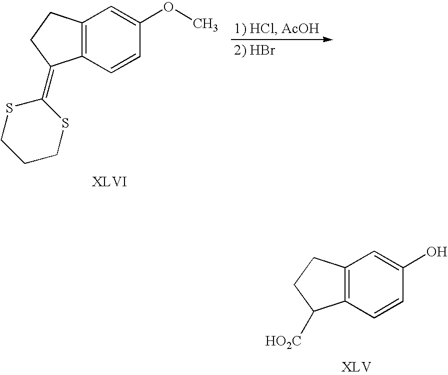

A compound of formula (XLVI) may be synthesized by reacting the anion of 1,3-dithiane with a compound of formula (XLVII). The resulting intermediate tertiary alcohol is then reacted with an acid like para-toluenesulfonic acid and heated in a solvent like toluene. A compound of formula (XLVII) may be purchased from commercial sources or may be made by literature procedures.

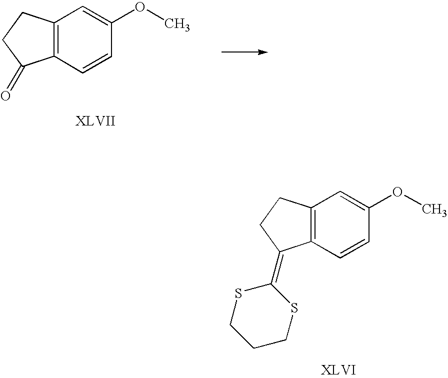

Based upon these examples and the disclosure contained herein one skilled in the art can readily convert compounds of formula (I) into other compounds of formula (I), or salts thereof. For example, an ester of a compound of formula (I) may be converted in an acid of a compound of formula (I) as in Examples 10-11, 13-17, 19 and 21.

The following examples are intended for illustration only and are not intended to limit the scope of the invention in any way, the present invention being defined by the claims.

In the examples, the following terms have the designated meaning:
aq=aqueous;
atm=atmosphere;
g=gram;
mg=milligram;
h=hour;
min=minute;
L=liter;
mL=milliliter;
M=molar;
mol=mole;
N=normal;
˜=approximately;
HPLC=high performance liquid chromatography;
NMR=nuclear magnetic resonance;
H=hydrogen atom;
Hz=Hertz; mHz=megaHertz;
DMSO=dimethylsulfoxide;

As used in the examples, 4-(Chloromethyl)-3-(2,6-dichlorophenyl)-5-(1-methylethyl)isoxazole may be prepared by a procedure similar to that described below:

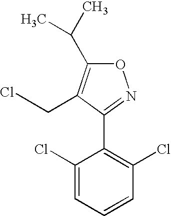

Thionyl chloride (123 mL, 202 g, 1.7 mol) was added dropwise during 30 min to a stirred suspension of benzotriazole (202 g, 1.7 mol) in dichloromethane (550 mL) at room temperature under N₂. The resulting yellow solution was transferred to an addition funnel and added dropwise during 1 hour to a stirred solution of [3-(2,6-dichlorophenyl)-5-(1-methylethyl)-4-isoxazolyl]methanol (372 g, 1.3 mol, Maloney, P. R., et al., 2000 J. Med. Chem. 43:2971-2974) in dichloromethane (975 mL). The reaction temperature gradually rose to a maximum of 28° C. After 1 hr the resulting suspension was filtered to remove the benzotriazole hydrochloride. The filtrate was washed with water (2×1 L), with 1 N NaOH (1 L), with water (1 L), dried over anhydrous Na₂SO₄, filtered and concentrated to yield 4-(chloromethyl)-3-(2,6-dichlorophenyl)-5-(1-methylethyl)isoxazole as a pale yellow oil (413 g, 80%). ¹H NMR (400 MHz, DMSO-d₆): δ 7.64 (m, 3H), 4.47 (s, 2H), 3.45 (m, 1H), 1.31 (d, J=7 Hz, 6H). ES-LCMS m/z 305 (M+H)⁺.

### Example 1

### 5-[4-({[3-(2,6-Dichlorophenyl)-5-(1-methylethyl)-4-isoxazolyl]methyl}oxy)phenyl]-1H-indole-2-carboxylic Acid

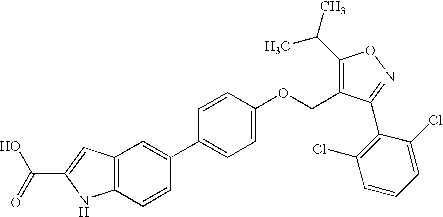

### 1a) 1-(1,1-Dimethylethyl) 2-ethyl 5-{[(trifluoromethyl)sulfonyl]oxy}-1H-indole-1,2-dicarboxylate

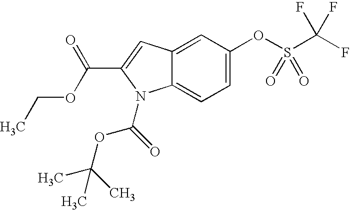

Bis(1,1-dimethylethyl) dicarbonate (1.78 g, 8.13 mmol) and N,N-dimethylaminopyridine (83 mg, 0.68 mmol) were added to ethyl 5-[(phenylmethyl)oxy]-1H-indole-2-carboxylate (2.0 g, 6.77 mmol) dissolved in tetrahydrofuran (20 mL) at room temperature. After stirring for 1.5 h, ethyl acetate was added and the mixture was washed with water and brine, then dried over sodium sulfate. This solution was concentrated, then the residue was taken up in methanol (40 mL) and chloroform (40 mL). Palladium on carbon (10%, 100 mg) was added and the mixture shaken in a Parr apparatus under hydrogen (40 psi) at room temperature for 1 h. The solution was filtered through a pad of Celite®, then concentrated. Dichloromethane (20 mL) was added and the solution cooled to 0° C. Triethylamine (1.95 mL, 13.8 mmol) and trifluoromethanesulfonic anhydride (930 μL, 5.54 mmol) were added and the solution stirred for 20 min. The solvent was evaporated and the residue taken up in ethyl acetate. This solution was washed with saturated sodium bicarbonate (aq) and brine, then concentrated. The residue was purified by silica gel chromatography eluting with 1:4 ethyl acetate:hexanes to give 1.12 g (38%) of 1-(1,1-dimethylethyl) 2-ethyl 5-{[(trifluoromethyl)sulfonyl]oxy}-1H-indole-1,2-dicarboxylate as a clear glass. ¹H NMR (400 MHz, CDCl₃): δ 8.15 (d, J=9 Hz, 1H), 7.51 (s, 1H), 7.29 (d, J=9 Hz, 1H), 7.07 (s, 1H), 4.39 (q, J=7 Hz, 2H), 1.62 (s, 9H), 1.39 (t, J=7 Hz, 3H). ESI-LCMS m/z 438 (M+H)⁺.

### 1b) 1-(1,1-Dimethylethyl) 2-ethyl 5-(4-hydroxyphenyl)-1H-indole-1,2-dicarboxylate

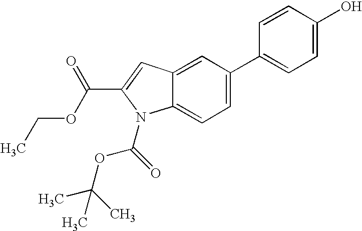

A mixture of 1-(1,1-dimethylethyl) 2-ethyl 5-{[(trifluoromethyl)sulfonyl]oxy}-1H-indole-1,2-dicarboxylate (660 mg, 1.51 mmol), 4-(4,4,5,5-tetramethyl-1,3,2-dioxaborolan-2-yl)phenol (830 mg, 3.77 mmol), palladium(II) acetate (17 mg, 0.08 mmol), triphenylphosphine (99 mg, 0.38 mmol), potassium phosphate (1.12 g, 5.28 mmol) and water (120 μL, 7.54 mmol) in dioxane (8 mL) was stirred at 130° C. for 1 h in a sealed tube. The mixture was filtered through a pad of Celite® and the pad washed with ethyl acetate. The combined filtrates were washed with water and brine, then concentrated. The residue was purified by silica gel chromatography eluting with 2:5 ethyl acetate:hexanes to give 290 mg (50%) of 1-(1,1-dimethylethyl) 2-ethyl 5-(4-hydroxyphenyl)-1H-indole-1,2-dicarboxylate as a clear glass. ¹H NMR (400 MHz, CDCl₃): δ 8.09 (d, J=9 Hz, 1H), 7.71 (s, 1H), 7.58 (d, J=9 Hz, 1H), 7.49 (d, J=9 Hz, 2H), 7.12 (s, 1H), 6.92 (d, J=9 Hz, 2H), 4.98-4.77 (br s, 1H), 4.39 (q, J=7 Hz, 2H), 1.63 (s, 9H), 1.37 (t, J=7 Hz, 3H).

### 1c) 5-[4-({[3-(2,6-Dichlorophenyl)-5-(1-methylethyl)-4-isoxazolyl]methyl}oxy)phenyl]-1H-indole-2-carboxylic Acid

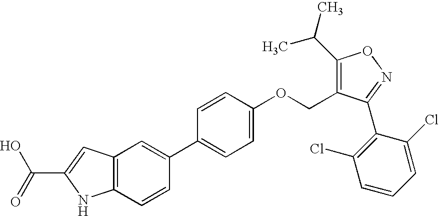

A mixture of 1-(1,1-dimethylethyl) 2-ethyl 5-(4-hydroxyphenyl)-1H-indole-1,2-dicarboxylate (150 mg, 0.39 mmol), 4-(chloromethyl)-3-(2,6-dichlorophenyl)-5-(1-methylethyl)isoxazole (144 mg, 0.47 mmol) and potassium carbonate (82 mg, 0.59 mmol) in N,N-dimethylformamide (2 mL) was stirred at room temperature for 16 h. Ethyl acetate was added and the mixture washed with water and brine, then concentrated. The residue was purified by silica gel chromatography eluting with 2:5 ethyl acetate:hexanes and the fractions containing product were combined and concentrated. To this residue was added sodium hydroxide (110 mg, 2.77 mmol) in a mixture of tetrahydrofuran (1 mL), ethyl alcohol (2 mL) and water (1 mL), then the solution was stirred at 50° C. for 3 h. The solution was concentrated to ⅓ volume, then added to a stirred solution of hydrochloric acid (0.5 M aq, 10 mL). The solution was extracted twice with ethyl acetate and the combined organics were washed with water and brine, then dried over sodium sulfate and concentrated to yield 96 mg (47%) of 5-[4-({[3-(2,6-dichlorophenyl)-5-(1-methylethyl)-4-isoxazolyl]methyl}oxy)phenyl]-1H-indole-2-carboxylic acid as a tan solid. ¹H NMR (400 MHz, d₆-DMSO): δ 12.93 (s, 1H), 11.75 (s, 1H), 7.78 (s, 1H), 7.61 (d, J=9 Hz, 1H), 7.55-7.40 (m, 5H), 7.08 (s, 1H), 6.82 (d, J=7 Hz, 2H), 4.82 (s, 2H), 3.44 (septet, J=7 Hz, 1H), 1.27 (d, J=7 Hz, 6H). ESI-LCMS m/z 521 (M+H)⁺.

### Example 2

### 6-[4-({[3-(2,6-Dichlorophenyl)-5-(1-methylethyl)-4-isoxazolyl]methyl}oxy)phenyl]-2,3-dihydro-1H-indene-1-carboxylic Acid

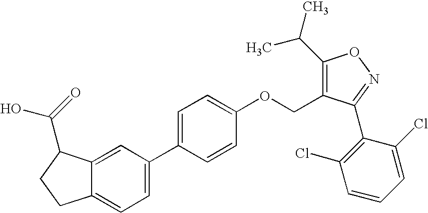

### 2a) Methyl 6-hydroxy-2,3-dihydro-1H-indene-1-carboxylate

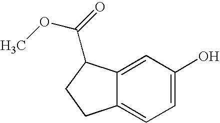

A solution of 1,3-dithiane (2.37 g, 19.7 mmol) in tetrahydrofuran (30 mL) was cooled to −15° C. and n-butyl lithium (2.5 M in hexanes, 7.40 mL, 18.5 mmol) was added. After stirring at −15° C. for 1 h, 6-(methyloxy)-2,3-dihydro-1H-inden-1-one (2.0 g, 12.3 mmol) in tetrahydrofuran (75 mL) was added dropwise, then the mixture was allowed to warm to room temperature over 3 h. Ethyl acetate was added and the mixture was washed with water and brine, then dried over sodium sulfate and concentrated. The residue was taken up in toluene (75 mL) and para-toluenesulfonic acid (350 mg, 1.85 mmol) was added. The mixture was heated at reflux in a Dean Stark apparatus for 1.5 h, then filtered through a plug of Celite®, and concentrated. The residue was purified by silica gel chromatography eluting with 1:3 ethyl acetate:hexanes to give 1.43 g (of a clear oil).

A portion of this oil (1.42 g) was heated at reflux in a mixture of acetic acid (25 mL) and hydrochloric acid (37% aq, 10 mL) for 16 h. Hydrobromic acid (30% in acetic acid, 20 mL) was added and the mixture heated at reflux for an additional 24 h. The mixture was concentrated to dryness and the residue was taken up in ethyl acetate, then washed with water and brine, then concentrated. The residue was taken up in methanol (40 mL) and thionyl chloride (790 μL, 10.7 mmol) was added dropwise. The solution was stirred at room temperature for 72 h. After concentration, the residue was purified by silica gel chromatography eluting with 2:5 ethyl acetate:hexanes to give 130 mg (13%) of methyl 6-hydroxy-2,3-dihydro-1H-indene-1-carboxylate as a clear glass. ¹H NMR (400 MHz, CDCl₃): δ 7.07 (d, J=8 Hz, 1H), 6.85 (s, 1H), 6.69 (d, J=8 Hz, 1H), 3.99 (dd, J=8, 7 Hz, 1H), 3.73 (s, 3H), 3.02-2.96 (m, 1H), 2.86-2.78 (m, 1H), 2.46-2.39 (m, 1H), 2.38-2.30 (m, 1H).

### 2b) Methyl 6-(4-hydroxyphenyl)-2,3-dihydro-1H-indene-1-carboxylate

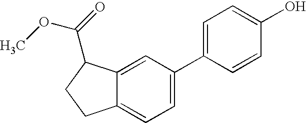

A solution of methyl 6-hydroxy-2,3-dihydro-1H-indene-1-carboxylate (130 mg, 0.68 mmol) in dichloromethane (4 mL) was cooled to 0° C. and triethylamine (240 μL, 1.69 mmol), then trifluoromethanesulfonic anhydride (140 μL, 0.81 mmol) were added dropwise. After stirring at room temperature for 30 min, the solution was concentrated and the residue taken up in ethyl acetate, washed with water and brine, then filtered through a pad of Celite®, and concentrated. The residue was taken up in dioxane (3 mL) and 4-(4,4,5,5-tetramethyl-1,3,2-dioxaborolan-2-yl)phenol (260 mg, 1.17 mmol), palladium(II) acetate (7 mg, 0.03 mmol), triphenylphosphine (16 mg, 0.06 mmol), potassium phosphate (435 mg, 2.05 mmol), and water (50 μL, 2.94 mmol) were added, and the mixture stirred at 90° C. for 20 min. The solution was filtered through a pad of Celite®, and the pad was washed with ethyl acetate. The combined filtrates were washed with water and brine, then concentrated. The residue was purified by silica gel chromatography eluting with 2:3 ethyl acetate:hexanes to give 84 mg (53%) of methyl 6-(4-hydroxyphenyl)-2,3-dihydro-1H-indene-1-carboxylate as a clear glass. ¹H NMR (400 MHz, CDCl₃): δ 7.52 (s, 1H), 7.44 (d, J=9 Hz, 2H), 7.38 (d, J=8 Hz, 1H), 7.28 (d, J=8 Hz, 1H), 6.87 (d, J=9 Hz, 2H), 4.83 (s, 1H), 4.12-4.08 (m, 1H), 3.74 (s, 3H), 3.16-3.08 (m, 1H), 2.97-2.89 (m, 1H), 2.51-2.40 (m, 1H), 2.39-2.34 (m, 1H).

2c) 6-[4-({[3-(2,6-Dichlorophenyl)-5-(1-methylethyl)-4-isoxazolyl]methyl}oxy)phenyl]-2,3-dihydro-1H-indene-1-carboxylic Acid

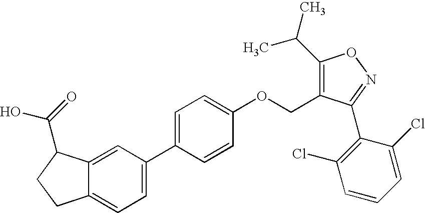

A solution of methyl 6-(4-hydroxyphenyl)-2,3-dihydro-1H-indene-1-carboxylate (84 mg, 0.31 mmol), 4-(chloromethyl)-3-(2,6-dichlorophenyl)-5-(1-methylethyl)isoxazole (190 mg, 0.63 mmol), and potassium carbonate (175 mg, 1.25 mmol) in N,N-dimethylformamide (2 mL) was stirred at 60° C. for 16 h. Ethyl acetate was added and the mixture washed with water and brine, then concentrated. The residue was purified by silica gel chromatography eluting with 2:3 ethyl acetate:hexanes and the fractions containing product were combined and concentrated. The residue was taken up in a mixture of tetrahydrofuran (1 mL), ethyl alcohol (3 mL), and water (1 mL), then sodium hydroxide (36 mg, 0.89 mmol) was added. After stirring at room temperature for 72 h, the solution was concentrated to ⅓ volume and poured into hydrochloric acid (1.0 M aq, 5 mL.) The resulting solids were collected by suction filtration, washed with water, then dried to give 36 mg (22%) of 6-[4-({[3-(2,6-dichlorophenyl)-5-(1-methylethyl)-4-isoxazolyl]methyl}oxy)phenyl]-2,3-dihydro-1H-indene-1-carboxylic acid as an off-white solid. ¹H NMR (400 MHz, d₆-DMSO): δ 7.60 (d, J=8 Hz, 2H), 7.54-7.48 (m, 2H), 7.38 (d, J=9 Hz, 2H), 7.34-7.32 (m, 1H), 7.23 (d, J=8 Hz, 1H), 6.82 (d, J=9 Hz, 2H), 4.81 (s, 2H), 3.94-3.90 (m, 1H), 3.47-3.41 (m, 1H), 2.95-2.90 (m, 1H), 2.83-2.78 (m, 1H), 2.27-2.19 (m, 2H), 1.31 (d, J=7 Hz, 6H). ESI-LCMS m/z 522 (M+H)⁺.

### Example 3

### 6-[4-({[3-(2,6-Dichlorophenyl)-5-(1-methylethyl)-4-isoxazolyl]methyl}oxy)phenyl]-1H-indole-3-carboxylic Acid

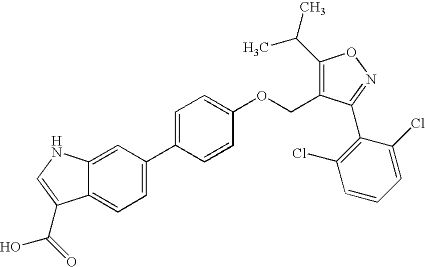

### 3a) 1-(1,1-Dimethylethyl) 3-ethyl 6-{[(trifluoromethyl)sulfonyl]oxy}-1H-indole-1,3-dicarboxylate

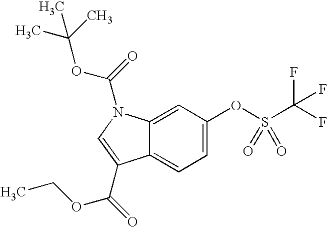

A mixture of ethyl 6-[(phenylmethyl)oxy]-1H-indole-3-carboxylate [may be prepared according to Bioorg. Med. Chem., 9 (8) 2119 (2001)] (350 mg, 1.19 mmol) and palladium on carbon (10%, 100 mg) in chloroform (10 mL) and methanol (5 mL) were stirred vigorously under hydrogen (1 atm) for 1.5 h. The mixture was filtered through a pad of Celite®, then concentrated. The residue was taken up in dichloromethane (8 mL), cooled to 0° C., and triethylamine (250 μL, 1.78 mmol), then trifluoromethanesulfonic anhydride (240 μL, 1.42 mmol) was added. After stirring at room temperature for 16 h the mixture was concentrated and the residue was taken up in ethyl acetate. The organics were washed with water and brine, then concentrated. The residue was taken up in tetrahydrofuran (4 mL). Bis(1,1-dimethylethyl)-dicarbonate (310 mg, 1.42 mmol) and N,N-dimethylaminopyridine (15 mg, 0.12 mmol) were added, then the mixture was stirred at room temperature for 16 h. Ethyl acetate was added. The solution was washed with water and brine, then concentrated. The residue was purified by silica gel chromatography eluting with 1:4 ethyl acetate:hexanes to give 265 mg (51%) of 1-(1,1-dimethylethyl) 3-ethyl 6-{[(trifluoromethyl)sulfonyl]oxy}-1H-indole-1,3-dicarboxylate as a pale yellow glass. ¹H NMR (400 MHz, CDCl₃): δ 8.33 (s, 1H), 8.21 (d, J=9 Hz, 1H), 8.16 (s, 1H), 7.27 (d, J=9 Hz, 1H), 4.40 (q, J=7 Hz, 2H), 1.69 (s, 9H), 1.42 (t, J=7 Hz, 3H).

### 3b) 1-(1,1-Dimethylethyl) 3-ethyl 6-(4-hydroxyphenyl)-1H-indole-1,3-dicarboxylate

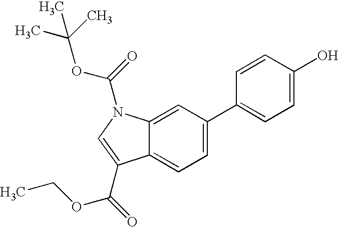

A mixture of 1-(1,1-dimethylethyl) 3-ethyl 6-{[(trifluoromethyl)sulfonyl]oxy}-1H-indole-1,3-dicarboxylate (265 mg, 0.61 mmol), 4-(4,4,5,5-tetramethyl-1,3,2-dioxaborolan-2-yl)phenol (270 mg, 1.21 mmol), palladium(II) acetate (7 mg, 0.03 mmol), triphenylphosphine (16 mg, 0.06 mmol), potassium phosphate (450 mg, 2.12 mmol) and water (50 μl, 3.03 mmol) in dioxane (3 mL) was stirred at 90° C. for 8 min. The mixture was filtered through a pad of Celite®, then the pad was washed with ethyl acetate. The combined filtrates were washed with water and brine, then concentrated. The residue was purified by silica gel chromatography eluting with 1:2 ethyl acetate:hexanes to give 140 mg (61%) of 1-(1,1-dimethylethyl) 3-ethyl 6-(4-hydroxyphenyl)-1H-indole-1,3-dicarboxylate as a white solid. ¹H NMR (400 MHz, CDCl₃): δ 8.39 (s, 1H), 8.25 (s, 1H), 8.15 (d, J=8 Hz, 1H), 7.57-7.53 (m, 3H), 6.92 (d, J=9 Hz, 2H), 4.41 (q, J=7 Hz, 2H), 1.69 (s, 9H), 1.42 (t, J=7 Hz, 3H). ESI-LCMS m/z 382 (M+H)⁺.

### 3c) 6-[4-({[3-(2,6-Dichlorophenyl)-5-(1-methylethyl)-4-isoxazolyl]methyl}oxy)phenyl]-1H-indole-3-carboxylic Acid

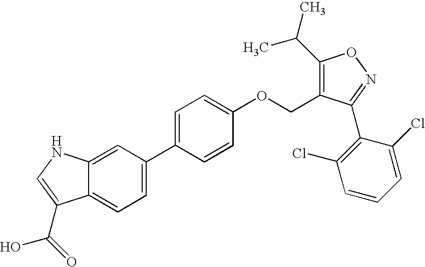

A solution of 1-(1,1-dimethylethyl) 3-ethyl 6-(4-hydroxyphenyl)-1H-indole-1,3-dicarboxylate (135 mg, 0.35 mmol), 4-(chloromethyl)-3-(2,6-dichlorophenyl)-5-(1-methylethyl)isoxazole (215 mg, 0.71 mmol) and potassium carbonate (200 mg, 1.42 mmol) in N,N-dimethylformamide (2 mL) was stirred at 60° C. for 16 h. Ethyl acetate was added and the mixture washed with water and brine, then concentrated. The residue was purified by silica gel chromatography eluting with 1:3 ethyl acetate:hexanes and the fractions containing product were combined and concentrated. The residue was taken up in a mixture of tetrahydrofuran (1 mL), ethyl alcohol (3 mL), and water (1 mL), then sodium hydroxide (80 mg, 1.92 mmol) was added. The solution was stirred at 50° C. for 16 h, then additional sodium hydroxide (40 mg, 0.96 mmol) and water (0.5 mL) were added and the mixture stirred at 65° C. for an additional 24 h. The solution was concentrated to ⅓ volume and the pH was adjusted to 6.0 with hydrochloric acid (1.0 M aq.). The aqueous solution was extracted twice with ethyl acetate and the combined extracts were washed with water and brine, then concentrated. The residue was taken up in a minimum of methanol, then added to water. The resulting solids were collected by suction filtration, washed with water, then dried to give 52 mg (28%) of 6-[4-({[3-(2,6-dichlorophenyl)-5-(1-methylethyl)-4-isoxazolyl]methyl}oxy)phenyl]-1H-indole-3-carboxylic acid as a beige solid. ¹H NMR (400 MHz, d₆-DMSO): δ 11.93 (s, 1H), 11.80 (s, 1H), 7.99-7.97 (m, 2H), 7.62 (d, J=8 Hz, 2H), 7.55-7.49 (m, 4H), 7.35 (d, J=8 Hz, 1H), 6.85 (d, J=9 Hz, 2H), 4.83 (s, 2H), 3.45 (septet, J=7 Hz, 1H), 1.32 (d, J=7 Hz, 6H). ESI-LCMS m/z 521 (M+H)⁺.

### Example 4

### 5-[4-({[3-(2,6-Dichlorophenyl)-5-(1-methylethyl)-4-isoxazolyl]methyl}oxy)phenyl]-1-benzofuran-2-carboxylic Acid

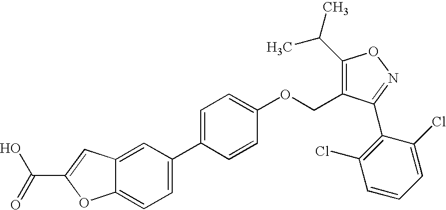

### 4a) Methyl 5-hydroxy-1-benzofuran-2-carboxylate

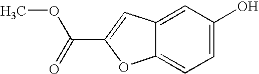

A solution of 5-(methyloxy)-1-benzofuran-2-carboxylic acid (2.0 g, 10.4 mmol) in dichloromethane (25 mL) was cooled to −15° C., then boron tribromide (1.0 M in dichloromethane, 26.0 mL, 26.0 mmol) was added dropwise. After stirring at −15° C. for 45 min, the reaction was quenched with saturated sodium bicarbonate (aq), acidified with hydrochloric acid (1.0 M, aq), then extracted twice with ethyl acetate. The combined extracts were washed with water and brine, then dried over sodium sulfate and concentrated. The residue was taken up in methanol (75 mL) and thionyl chloride (2.27 mL, 31.2 mmol) was added dropwise. The solution was stirred at 80° C. for 1 h, then concentrated. The residue was taken up in ethyl acetate, then washed with saturated sodium bicarbonate (aq), water, and brine, then dried over sodium sulfate and concentrated to give 1.91 g (95%) of methyl 5-hydroxy-1-benzofuran-2-carboxylate as a white solid. ¹H NMR (400 MHz, d₆-DMSO): δ 9.46 (s, 1H), 7.59 (s, 1H), 7.47 (d, J=9 Hz, 1H), 7.03 (s, 1H), 6.96 (d, J=9 Hz, 1H), 3.84 (s, 3H).

### 4b) Methyl 5-{[(trifluoromethyl)sulfonyl]oxy}-1-benzofuran-2-carboxylate

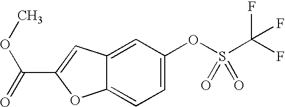

A solution of methyl 5-hydroxy-1-benzofuran-2-carboxylate (750 mg, 3.90 mmol) in dichloromethane (10 mL) was cooled to 0° C., then triethylamine (820 μl, 5.85 mmol) and trifluoromethanesulfonic anhydride (790 μL, 4.68 mmol) were added and the solution stirred at room temperature for 1 h. After concentration, the residue was taken up in ethyl acetate and washed with water and brine, then dried over sodium sulfate and concentrated. The residue was purified by silica gel chromatography eluting with 1:2 ethyl acetate:hexanes to give 1.12 g (33%) of methyl 5-{[(trifluoromethyl)sulfonyl]oxy}-1-benzofuran-2-carboxylate as a white solid. ¹H NMR (400 MHz, CDCl₃): δ 7.66-7.62 (m, 2H), 7.54 (s, 1H), 7.36 (dd, J=8, 2 Hz, 1H), 3.99 (s, 3H). ESI-LCMS m/z 325 (M+H)⁺.

### 4c) Methyl 5-(4-hydroxyphenyl)-1-benzofuran-2-carboxylate

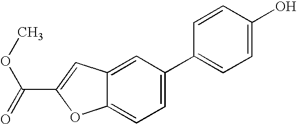

A mixture of methyl 5-{[(trifluoromethyl)sulfonyl]oxy}-1-benzofuran-2-carboxylate (300 mg, 0.93 mmol), 4-(4,4,5,5-tetramethyl-1,3,2-dioxaborolan-2-yl)phenol (510 mg, 2.31 mmol), palladium(II) acetate (10 mg, 0.05 mmol), triphenylphosphine (61 mg, 0.23 mmol), potassium phosphate (690 mg, 3.24 mmol) and water (75 μL, 4.63 mmol) in dioxane (4 mL) was stirred at 100° C. for 15 min. The mixture was filtered through a pad of Celite®, then the pad was washed with ethyl acetate. The combined filtrates were washed with water and brine, then concentrated. The residue was purified by silica gel chromatography eluting with 3:2 ethyl acetate:hexanes to give 98 mg (40%) of methyl 5-(4-hydroxyphenyl)-1-benzofuran-2-carboxylate as a white solid. ESI-LCMS m/z 269 (M+H)⁺.

### 4d) 5-[4-({[3-(2,6-Dichlorophenyl)-5-(1-methylethyl)-4-isoxazolyl]methyl}oxy)phenyl]-1-benzofuran-2-carboxylic Acid

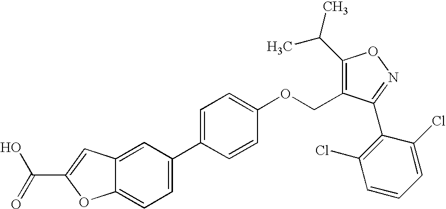

A solution of methyl 5-(4-hydroxyphenyl)-1-benzofuran-2-carboxylate (135 mg, from multiple batches, 0.50 mmol), 4-(chloromethyl)-3-(2,6-dichlorophenyl)-5-(1-methylethyl)isoxazole (305 mg, 1.00 mmol), and potassium carbonate (280 mg, 2.01 mmol) in N,N-dimethylformamide (2 mL) was stirred at 60° C. for 16 h. Ethyl acetate was added and the mixture washed with water and brine, then concentrated. The residue was purified by silica gel chromatography eluting with 2:3 ethyl acetate:hexanes and the fractions containing product were combined and concentrated. The residue was taken up in a mixture of tetrahydrofuran (2 mL), ethyl alcohol (5 mL), and water (2 mL), then sodium hydroxide (160 mg, 4.01 mmol) was added. The solution was stirred at 50° C. for 16 h, then concentrated to ⅓ volume and poured into hydrochloric acid (1.0 M aq, 5 mL). The solution was extracted twice with ethyl acetate and the combined extracts washed with water and brine, then dried over sodium sulfate and concentrated. The resulting solids were recrystrallized from ethyl acetate/hexanes to give 132 mg (50%) of 5-[4-({[3-(2,6-Dichlorophenyl)-5-(1-methylethyl)-4-isoxazolyl]methyl}oxy)phenyl]-1-benzofuran-2-carboxylic acid as an pinkish-white solid. ¹H NMR (400 MHz, d₆-DMSO): δ 13.55 (s, 1H), 7.91 (s, 1H), 7.72-7.60 (m, 5H), 7.55-7.51 (m, 3H), 6.86 (d, J=9 Hz, 2H), 4.48 (s, 2H), 3.44 (septet, J=7 Hz, 1H), 1.32 (d, J=7 Hz, 6H). ESI-LCMS m/z 522 (M+H)⁺.

### Example 5

### 6-[4-({[3-(2,6-Dichlorophenyl)-5-(1-methylethyl)-4-isoxazolyl]methyl}oxy)phenyl]-1H-indole-2-carboxylic Acid

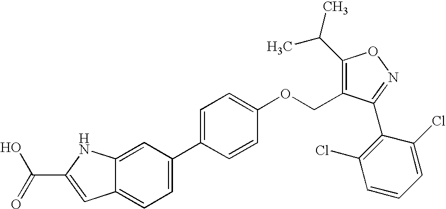

### 5a) Methyl 6-(hydroxy)-1H-indole-2-carboxylate

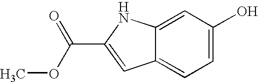

Boron tribromide (1.0 M in dichloromethane, 14.6 mL, 14.6 mmol) was added dropwise to a solution of methyl 6-(methyloxy)-1H-indole-2-carboxylate (1.0 g, 4.87 mmol) in dichloromethane (10 mL) at 0° C., and the resulting mixture was stirred at room temperature for 2 h. Boron tribromide (1.0 M in dichloromethane, 14.6 mL, 14.6 mmol) was added and the mixture stirred for 16 h. The mixture was poured into saturated sodium bicarbonate (aq, 150 mL), then the pH was adjusted to 6 with hydrochloric acid (37% aq). The solution was extracted three times with ethyl acetate, then the combined extracts were washed with water and brine, then concentrated. The residue was taken up in methanol (20 mL), and thionyl chloride (1.06 mL, 14.6 mmol) was added. The solution was heated at reflux for 1 h, then concentrated, and the residue was purified by silica gel chromatography eluting with 3:2 ethyl acetate:hexanes to give 580 mg (62%) of methyl 6-(hydroxy)-1H-indole-2-carboxylate as a white solid. ¹H NMR (400 MHz, d₆-DMSO): δ 11.44 (s, 1H), 9.34 (s, 1H), 7.40 (d, J=9 Hz, 1H), 7.00 (s, 1H), 6.75 (s, 1H), 6.58 (d, J=9 Hz, 1H), 3.80 (s, 3H).

### 5b) 1-(1,1-Dimethylethyl) 2-methyl 6-{[(trifluoromethyl)sulfonyl]oxy}-1H-indole-1,2-dicarboxylate

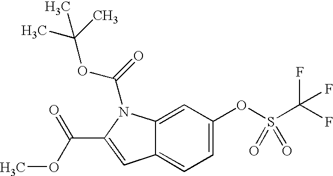

Trifluoromethanesulfonic anhydride (880 μL, 5.21 mmol) was added to a solution of methyl 6-(hydroxy)-1H-indole-2-carboxylate (830 mg, from multiple batches, 4.34 mmol) and triethylamine (910 μL, 6.51 mmol) in dichloromethane (40 mL) at 0° C., then the reaction was stirred for 30 min at room temperature. The solution was concentrated and the residue was taken up in ethyl acetate, washed with water and brine, then dried over sodium sulfate and concentrated. The residue was taken up in dichloromethane (10 mL) and then bis(1,1-dimethylethyl) dicarbonate (1.14 g, 5.21 mmol) and N,N-dimethylaminopyridine (55 mg, 0.43 mmol) were added. After stirring at room temperature for 16 h, ethyl acetate was added, and the solution was washed with water and brine, then concentrated. The residue was purified by silica gel chromatography eluting with 3:1 ethyl acetate:hexanes to give 1.42 g (77%) of 1-(1,1-dimethylethyl) 2-methyl 6-{[(trifluoromethyl)sulfonyl]oxy}-1H-indole-1,2-dicarboxylate as a clear viscous oil. ¹H NMR (400 MHz, CDCl₃): δ 8.07 (s, 1H), 7.67 (d, J=9 Hz, 1H), 7.20 (d, J=9 Hz, 1H), 7.08 (s, 1H), 3.93 (s, 3H), 1.63 (s, 9H).

### 5c) 1-(1,1-Dimethylethyl) 2-methyl 6-(4-hydroxyphenyl)-1H-indole-1,2-dicarboxylate

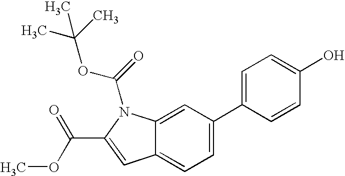

A mixture of 1-(1,1-dimethylethyl) 2-methyl 6-{[(trifluoromethyl)sulfonyl]oxy}-1H-indole-1,2-dicarboxylate (330 mg, 0.78 mmol), 4-(4,4,5,5-tetramethyl-1,3,2-dioxaborolan-2-yl)phenol (340 mg, 1.56 mmol), palladium(II) acetate (9 mg, 0.04 mmol), triphenylphosphine (51 mg, 0.19 mmol), potassium phosphate (580 mg, 2.73 mmol) and water (65 μl, 3.90 mmol) in dioxane (4 mL) was stirred at 100° C. for 30 min. The mixture was filtered through a pad of Celite®, then the pad was washed with ethyl acetate. The combined filtrates were washed with water and brine, then concentrated. The residue was purified by silica gel chromatography eluting with 3:2 ethyl acetate:hexanes to give 55 mg (19%) of 1-(1,1-dimethylethyl) 2-methyl 6-(4-hydroxyphenyl)-1H-indole-1,2-dicarboxylate as a white solid. ¹H NMR (400 MHz, CDCl₃): δ 8.29 (s, 1H), 7.62 (d, J=8 Hz, 1H), 7.55 (d, J=9 Hz, 2H), 7.46 (d, J=8 Hz, 1H), 7.11 (s, 1H), 6.92 (d, J=9 Hz, 2H), 3.92 (s, 3H), 1.62 (s, 9H).

### 5d) 6-[4-({[3-(2,6-Dichlorophenyl)-5-(1-methylethyl)-4-isoxazolyl]methyl}oxy)phenyl]-1H-indole-2-carboxylic Acid

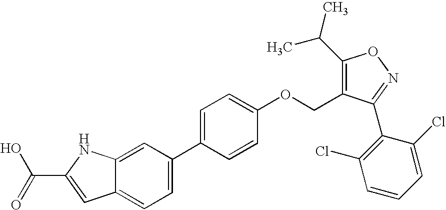

A mixture of 1-(1,1-dimethylethyl) 2-methyl 6-(4-hydroxyphenyl)-1H-indole-1,2-dicarboxylate (53 mg, 0.14 mmol), 4-(chloromethyl)-3-(2,6-dichlorophenyl)-5-(1-methylethyl)isoxazole (88 mg, 0.29 mmol), and potassium carbonate (80 mg, 0.58 mmol) in N,N-dimethylformamide (1 mL) was stirred at 60° C. for 16 h. Ethyl acetate was added, and the mixture washed with water and brine, then concentrated. The residue was purified by silica gel chromatography eluting with 1:5 ethyl acetate:hexanes and the fractions containing product were combined and concentrated. The residue was taken up in a mixture of tetrahydrofuran (1 mL), ethyl alcohol (3 mL), and water (1 mL), then sodium hydroxide (56 mg, 1.40 mmol) was added. The solution was stirred at 50° C. for 16 h, then concentrated to ½ volume. The pH was adjusted to 6.0 with hydrochloric acid (1.0 M aq), then the solution was extracted twice with ethyl acetate. The combined extracts were washed with water and brine, then dried over sodium sulfate and concentrated to give 49 mg (65%) of 6-[4-({[3-(2,6-dichlorophenyl)-5-(1-methylethyl)-4-isoxazolyl]methyl}oxy)phenyl]-1H-indole-2-carboxylic acid as a beige powder. ¹H NMR (400 MHz, d₆-DMSO): δ 12.91 (s, 1H), 11.74 (s, 1H), 7.66-7.60 (m, 3H), 7.55-7.45 (m, 4H), 7.26 (dd, J=8, 1 Hz, 1H), 7.06 (d, J=1 Hz, 1H), 6.87 (d, J=9 Hz, 2H), 4.83 (s, 2H), 3.45 (septet, J=7 Hz, 1H), 1.32 (d, J=7 Hz, 6H). ESI-LCMS m/z 521 (M+H)⁺.

### Example 6

### 5-[4-({[3-(2,6-Dichlorophenyl)-5-(1-methylethyl)-4-isoxazolyl]methyl}oxy)phenyl]-2,3-dihydro-1H-indene-2-carboxylic Acid

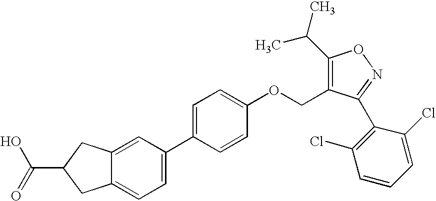

### 6a) Methyl 5-bromo-2,3-dihydro-1H-indene-2-carboxylate

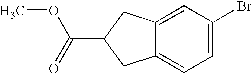

N-Bromosuccinimide (4.35 g, 24.4 mmol) and benzoyl peroxide (150 mg) were added to 4-bromo-1,2-dimethylbenzene (1.5 mL, 11.1 mmol) in carbon tetrachloride (30 mL), then stirred at 80° C. for 6 h. N-Bromosuccinimide (990 mg, 0.55 mmol) was added and the mixture was stirred at 80° C. for 16 h. The solution was washed with water and brine, then concentrated. The residue was purified by silica gel chromatography eluting with 1:10 ethyl acetate:hexanes and the fractions containing product were combined and concentrated. The residue was taken up in tetrahydrofuran (5 mL) and added to a solution of sodium hydride (440 mg of 60%, 10.9 mmol) and dimethylmalonate (560 μL, 4.96 mmol) in tetrahydrofuran (10 mL) which had been prestirred for 30 min at room temperature. The combined solution was stirred at room temperature for 16 h. Ethyl acetate was added and the solution was washed with water and brine, then concentrated. The residue was purified by silica gel chromatography eluting with 2:3 ethyl acetate:hexanes to give 170 mg (13%) of methyl 5-bromo-2,3-dihydro-1H-indene-2-carboxylate as a white solid. ¹H NMR (400 MHz, CDCl₃): δ 7.33 (s, 1H), 7.25 (d, J=8 Hz, 1H), 7.06 (d, J=8 Hz, 1H), 3.72 (s, 3H), 3.36-3.32 (m, 1H), 3.23-3.14 (m, 4H). ESI-LCMS m/z 256 (M+H)⁺.

### 6b) Methyl 5-(4-hydroxyphenyl)-2,3-dihydro-1H-indene-2-carboxylate

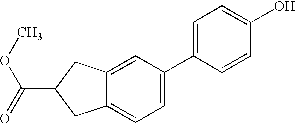

A mixture of methyl 5-bromo-2,3-dihydro-1H-indene-2-carboxylate (170 mg, 0.67 mmol), 4-(4,4,5,5-tetramethyl-1,3,2-dioxaborolan-2-yl)phenol (290 mg, 1.33 mmol), palladium(II) acetate (7 mg, 0.03 mmol), triphenylphosphine (17 mg, 0.07 mmol), potassium phosphate (495 mg, 2.33 mmol) and water (55 μL, 3.33 mmol) in dioxane (3 mL) was stirred at 90° C. for 6 h. The mixture was filtered through a pad of Celite®, then the pad was washed with ethyl acetate. The combined filtrates were washed with water and brine, then concentrated. The residue was purified by silica gel chromatography eluting with 1:1 ethyl acetate:hexanes to give 88 mg (50%) of methyl 5-(4-hydroxyphenyl)-2,3-dihydro-1H-indene-2-carboxylate as a beige solid. ¹H NMR (400 MHz, CDCl₃): δ 7.43 (d, J=7 Hz, 2H), 7.42 (s, 1H), 7.37-7.32 (m, 1H), 7.23 (s, 1H), 6.86 (d, J=7 Hz, 2H), 4.82 (s, 1H), 3.74 (s, 3H), 3.40-3.34 (m, 1H), 3.31-3.24 (m, 4H).

### 6c) 5-[4-({[3-(2,6-Dichlorophenyl)-5-(1-methylethyl)-4-isoxazolyl]methyl}oxy)phenyl]-2,3-dihydro-1H-indene-2-carboxylic Acid

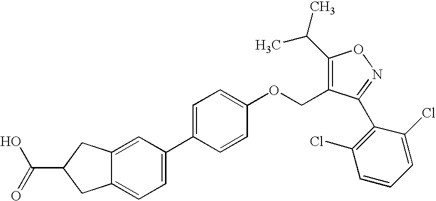

A solution of methyl 5-(4-hydroxyphenyl)-2,3-dihydro-1H-indene-2-carboxylate (85 mg, 0.32 mmol), 4-(chloromethyl)-3-(2,6-dichlorophenyl)-5-(1-methylethyl)isoxazole (195 mg, 0.63 mmol), and potassium carbonate (175 mg, 1.27 mmol) in N,N-dimethylformamide (2 mL) was stirred at 50° C. for 16 h. Ethyl acetate was added and the mixture washed with water and brine, then concentrated. The residue was purified by silica gel chromatography eluting with 1:3 ethyl acetate:hexanes and the fractions containing product were combined and concentrated. The residue was taken up in a mixture of tetrahydrofuran (1 mL), ethyl alcohol (3 mL), and water (1 mL), then sodium hydroxide (90 mg, 2.22 mmol) was added. The solution was stirred at 50° C. for 16 h, then concentrated to ⅓ volume and added dropwise to hydrochloric acid (1.0 M aq, 5 mL). The resulting solids were collected by suction filtration, washed with water, then dried to give 79 mg (48%) of 5-[4-({[3-(2,6-dichlorophenyl)-5-(1-methylethyl)-4-isoxazolyl]methyl}oxy)phenyl]-2,3-dihydro-1H-indene-2-carboxylic acid as an light gray solid. ¹H NMR (400 MHz, d₆-DMSO): δ 7.61 (d, J=8 Hz, 2H), 7.54-7.50 (m, 1H), 7.44 (d, J=9 Hz, 2H), 7.37 (s, 1H), 7.30 (d, J=8 Hz, 1H), 7.21 (d, J=8 Hz, 1H), 6.82 (d, J=9 Hz, 2H), 4.81 (s, 2H), 3.43 (septet, J=7 Hz, 1H), 3.40-3.22 (m, 3H), 3.13-3.09 (m, 2H), 1.31 (d, J=7 Hz, 6H). ESI-LCMS m/z 522 (M+H)⁺.

### Example 7

### 5-[4-({[3-(2,6-Dichlorophenyl)-5-(1-methylethyl)-4-isoxazolyl]methyl}oxy)phenyl]-1-hydroxy-2,3-dihydro-1H-indene-1-carboxylic Acid

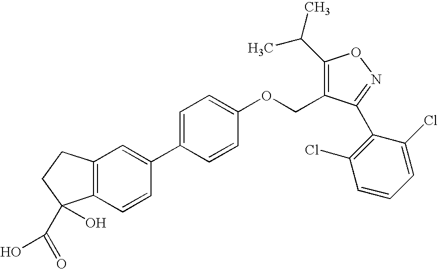

### 7a) Methyl 5-{[(trifluoromethyl)sulfonyl]oxy}-2,3-dihydro-1H-indene-1-carboxylate

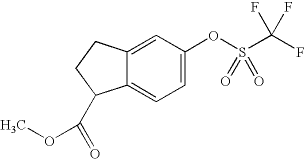

A solution of 1,3-dithiane (2.37 g, 19.7 mmol) in tetrahydrofuran (30 mL) was cooled to −15° C. and n-butyl lithium (2.5 M in hexanes, 7.40 mL, 18.5 mmol) was added. After stirring at −15° C. for 1 h, 5-(methyloxy)-2,3-dihydro-1H-inden-1-one (2.0 g, 12.3 mmol) in tetrahydrofuran (75 mL) was added dropwise, then the mixture was allowed to warm to room temperature over 4 h. Ethyl acetate was added and the mixture was washed with water and brine, then dried over sodium sulfate and concentrated. The residue was taken up in toluene (75 mL) and para-toluenesulfonic acid (350 mg, 1.85 mmol) was added. The mixture was heated at reflux in a Dean Stark apparatus for 2 h, then filtered through a plug of Celite®, and concentrated. The residue was purified by silica gel chromatography eluting with 1:4 ethyl acetate:hexanes to give 2.24 g (69%) of a viscous oil. A solution of a portion of this oil (1.42 g) in a mixture of acetic acid (25 mL) and hydrochloric acid (37% aq, 10 mL) was heated at reflux for 16 h. Hydrobromic acid (30% in acetic acid, 20 mL) was added and the mixture heated at reflux for an additional 24 h. The mixture was concentrated to dryness and the residue was taken up in toluene, then evaporated and dried in vacuo. The residue was taken up in methanol (50 mL), then thionyl chloride (3.05 mL, 41.8 mmol) was added dropwise. The solution was heated at reflux for 16 h. The solution was filtered through a plug of Celite®, then concentrated. The residue was purified by silica gel chromatography eluting with 1:2 ethyl acetate:hexanes and the fractions containing product were combined and concentrated. The residue was taken up in dichloromethane (2 mL), then cooled to 0° C. Triethylamine (240 μL, 1.69 mmol) and trifluoromethylsulfonic anhydride (140 μL, 0.81 mmol) were added and the solution stirred at room temperature for 16 h. The solution was washed with water and brine, then dried over sodium sulfate and concentrated to give 205 mg (8%) of methyl 5-{[(trifluoromethyl)sulfonyl]oxy}-2,3-dihydro-1H-indene-1-carboxylate as a beige oil. ¹H NMR (400 MHz, CDCl₃): δ 7.43 (d, J=9 Hz, 1H), 7.13 (s, 1H), 7.07 (d, J=9 Hz, 1H), 4.50 (t, J=8 Hz, 1H), 3.74 (s, 3H), 3.16-3.08 (m, 1H), 2.98-2.91 (m, 1H), 2.49-2.36 (m, 2H).

### 7b) Methyl 5-(4-hydroxyphenyl)-2,3-dihydro-1H-indene-1-carboxylate

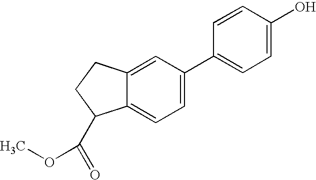

A mixture of methyl 5-{[(trifluoromethyl)sulfonyl]oxy}-2,3-dihydro-1H-indene-1-carboxylate (200 mg, 0.62 mmol), 4-(4,4,5,5-tetramethyl-1,3,2-dioxaborolan-2-yl)phenol (270 mg, 1.23 mmol), palladium(II) acetate (7 mg, 0.03 mmol), triphenylphosphine (16 mg, 0.06 mmol), potassium phosphate (460 mg, 2.16 mmol) and water (50 μL, 3.08 mmol) in dioxane (3 mL) was stirred at 90° C. for 30 min. The mixture was filtered through a pad of Celite®, then the pad was washed with ethyl acetate. The combined filtrates were washed with water and brine, then concentrated. The residue was purified by silica gel chromatography eluting with 2:3 ethyl acetate:hexanes to give 71 mg (43%) of methyl 5-(4-hydroxyphenyl)-2,3-dihydro-1H-indene-1-carboxylate as a clear glass. ¹H NMR (400 MHz, CDCl₃): δ 7.45-7.39 (m, 4H), 7.33 (s, 1H), 6.86 (d, J=8 Hz, 2H), 4.83 (s, 1H), 4.08 (t, J=8 Hz, 1H), 3.75 (s, 3H), 3.19-3.11 (m, 1H), 3.00-2.91 (m, 1H), 2.50-2.38 (m, 2H). ESI-LCMS m/z 269 (M+H)⁺.

### 7c) 5-[4-({[3-(2,6-Dichlorophenyl)-5-(1-methylethyl)-4-isoxazolyl]methyl}oxy)phenyl]-1-hydroxy-2,3-dihydro-1H-indene-1-carboxylic Acid

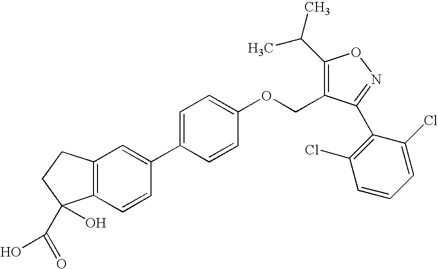

A solution of methyl 5-(4-hydroxyphenyl)-2,3-dihydro-1H-indene-1-carboxylate (69 mg, 0.26 mmol), 4-(chloromethyl)-3-(2,6-dichlorophenyl)-5-(1-methylethyl)isoxazole (157 mg, 0.51 mmol), and potassium carbonate (142 mg, 1.03 mmol) in N,N-dimethylformamide (2 mL) was stirred at room temperature for 72 h. 4-(chloromethyl)-3-(2,6-dichlorophenyl)-5-(1-methylethyl)isoxazole (80 mg, 0.26 mmol) and potassium carbonate (70 mg, 0.52 mmol) were added and the mixture stirred at 60° C. for 16 h. Ethyl acetate was added and the mixture washed with water and brine, then concentrated. The residue was purified by silica gel chromatography eluting with 2:3 ethyl acetate:hexanes and the fractions containing product were combined and concentrated. The residue was taken up in a mixture of tetrahydrofuran (1 mL), ethyl alcohol (3 mL), and water (1 mL), then sodium hydroxide (40 mg, 1.00 mmol) was added. The solution was stirred at 50° C. for 16 h, then concentrated to ⅓ volume and added dropwise to hydrochloric acid (1.0 M aq, 5 mL). The resulting solids were collected by suction filtration, washed with water, then dried to give 39 mg (29%) of 5-[4-({[3-(2,6-dichlorophenyl)-5-(1-methylethyl)-4-isoxazolyl]methyl}oxy)phenyl]-1-hydroxy-2,3-dihydro-1H-indene-1-carboxylic acid as a tan solid. ¹H NMR (400 MHz, d₆-DMSO): δ 12.52 (s, 1H), 7.61 (d, J=8 Hz, 2H), 7.54-7.52 (m, 1H), 7.49 (d, J=7 Hz, 2H), 7.46-7.40 (m, 2H), 7.36 (d, J=9 Hz, 1H), 6.83 (d, J=9 Hz, 2H), 4.82 (s, 2H), 3.42 (septet, J=7 Hz, 1H), 3.01-2.90 (m, 2H), 2.61-2.55 (m, 1H), 2.08-2.01 (m, 1H), 1.31 (d, J=7 Hz, 6H). ESI-LCMS m/z 538 (M+H)⁺.

### Example 8

### 6-[4-({[3-(2,6-Dichlorophenyl)-5-(1-methylethyl)-4-isoxazolyl]methyl}oxy)phenyl]-1H-indazole-3-carboxylic Acid

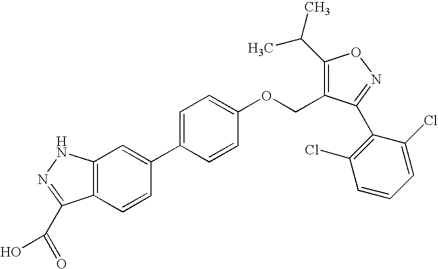

### 8a) Methyl (4-bromo-2-nitrophenyl)acetate

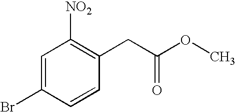

Dimethylmalonate (2.45 mL, 21.8 mmol) was added over 10 min at room temperature to a stirred suspension of sodium hydride (1.06 g of a 60% dispersion in mineral oil, 26.7 mmol) in 1-methyl-2-pyrrolidinone (25 mL). After stirring for an additional 15 min at room temperature, the solution was cooled to 0° C. and 2,5-dibromonitrobenzene (5.0 g, 17.8 mmol) was added and the mixture stirred at room temperature for 16 h. The mixture was poured into hydrochloric acid (1.0 M aq, 250 mL), then extracted with ethyl acetate twice. The combined extracts were washed with water and brine, then concentrated. The residue was purified by silica gel chromatography eluting with 3:2 ethyl acetate:hexanes and the fractions containing product were combined and concentrated. The residue was taken up in a mixture of 1-methyl-2-pyrrolidinone (20 mL) and water (2 mL), then stirred at 120° C. for 5 h. The mixture was poured into hydrochloric acid (1.0 M aq, 250 mL), then extracted with ethyl acetate twice. The combined extracts were washed with water and brine, then concentrated. The residue was purified by silica gel chromatography eluting with 2:3 ethyl acetate:hexanes to give 2.51 g (51%) of methyl (4-bromo-2-nitrophenyl)acetate as a pale yellow oil which solidified upon standing. ¹H NMR (400 MHz, CDCl₃): δ 8.26 (s, 1H), 7.71 (d, J=8 Hz, 1H), 7.24 (d, J=8 Hz, 1H), 3.98 (s, 2H), 3.71 (s, 3H). ESI-LCMS m/z 275 (M+H)⁺.

### 8b) Methyl {3-nitro-4′-[(phenylmethyl)oxy]-4-biphenylyl}acetate

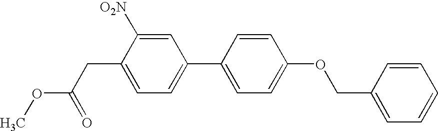

A mixture of methyl (4-bromo-2-nitrophenyl)acetate (1.31 g, 4.78 mmol), {4-[(phenylmethyl)oxy]phenyl}boronic acid (1.64 g, 7.17 mmol), tetrakis(triphenylphosphine)-palladium (0) (550 mg, 0.48 mmol), and sodium carbonate (2.0 M aq, 8.40 mL) in 1,2-dimethoxyethane (30 mL) was stirred at 70° C. for 2 h. The mixture was filtered through a pad of Celite®, and the pad washed with ethyl acetate. The combined filtrates were concentrated and the residue was purified by silica gel chromatography eluting with 2:3 ethyl acetate:hexanes to give 1.49 g (82%) of methyl {3-nitro-4′-[(phenylmethyl)oxy]-4-biphenylyl}acetate as a pale yellow solid. ESI-LCMS m/z 378 (M+H)⁺.

### 8c) Methyl {3-(acetylamino)-4′-[(phenylmethyl)oxy]-4-biphenylyl}acetate

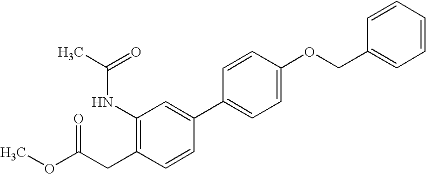

Methyl {3-nitro-4′-[(phenylmethyl)oxy]-4-biphenylyl}acetate (355 mg, 0.94 mmol) and palladium on carbon (10%, 200 mg) in a mixture of acetic acid (15 mL), acetic anhydride (1.5 mL) and water (100 μL) was shaken under a hydrogen atmosphere (30 psi) in a Parr apparatus at room temperature for 2 h. The mixture was filtered through a pad of Celite® and silica gel and the pad was washed with ethyl acetate. The combined extracts were washed with saturated sodium bicarbonate (aq) and brine, then dried over sodium sulfate and concentrated. The residue was taken up in N,N-dimethylformamide (4 mL). Benzyl bromide (100 μL, 0.84 mmol) and potassium carbonate (330 mg, 2.41 mmol) were added. The mixture was stirred at 40° C. for 16 h. Ethyl acetate was added and the mixture washed with water and brine, then dried over sodium sulfate and concentrated. The residue was purified by silica gel chromatography eluting with 3:1 ethyl acetate:hexanes to give 130 mg (36%) of methyl {3-(acetylamino)-4′-[(phenylmethyl)oxy]-4-biphenylyl}acetate as a beige solid. ESI-LCMS m/z 390 (M+H)⁺.

### 8d) 1-(1,1-Dimethylethyl) 3-methyl 6-(4-hydroxyphenyl)-1H-indazole-1,3-dicarboxylate

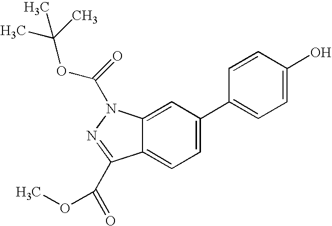

A solution of methyl {3-(acetylamino)-4′-[(phenylmethyl)oxy]-4-biphenylyl}acetate (130 mg, 0.33 mmol) and 1,1-dimethylethyl nitrite (60 μL, 0.50 mmol) in acetic acid (3 mL) was stirred at 80° C. for 16 h. After cooling to room temperature, the resulting solids were collected by suction filtration, washed with acetic acid and hexanes, then dried. Dichloromethane (1.5 mL) was added and the solution cooled to 0° C. Boron trichloride (1.0 M in dichloromethane, 230 μL, 0.23 mmol) was added and the solution stirred for 5 min. The reaction was quenched with methanol, then concentrated. The residue was taken up in ethyl acetate, then allowed to sit for 5 min. The solution was decanted away from the solids and the solids were then dried. The dried solids were dissolved in dichloromethane (2 mL). Bis(1,1-dimethylethyl) dicarbonate (51 mg, 0.23 mmol), triethylamine (80 μL, 0.58 mmol) and N,N-dimethylaminopyridine (2 mg, 0.02 mmol) were added and the solution stirred at room temperature for 16 h. The solution was concentrated and the residue taken up in ethyl acetate, then washed with water and brine, then dried over sodium sulfate and concentrated to give 78 mg (60%) of 1-(1,1-dimethylethyl) 3-methyl 6-(4-hydroxyphenyl)-1H-indazole-1,3-dicarboxylate as a beige solid. ¹H NMR (400 MHz, CDCl₃): δ 8.42 (s, 1H), 8.28 (d, J=9 Hz, 1H), 8.19 (s, 1H), 7.69-7.63 (m, 3H), 7.29 (d, J=9 Hz, 2H), 4.04 (s, 3H), 1.74 (s, 9H).

### 8e) 6-[4-({[3-(2,6-Dichlorophenyl)-5-(1-methylethyl)-4-isoxazolyl]methyl}oxy)phenyl]-1H-indazole-3-carboxylic Acid

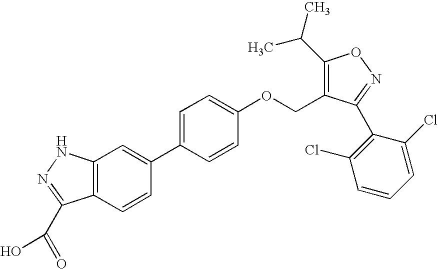

A solution of 1-(1,1-dimethylethyl) 3-methyl 6-(4-hydroxyphenyl)-1H-indazole-1,3-dicarboxylate (71 mg, 0.19 mmol), 4-(chloromethyl)-3-(2,6-dichlorophenyl)-5-(1-methylethyl)isoxazole (120 mg, 0.39 mmol), and potassium carbonate (94 mg, 0.68 mmol) in N,N-dimethylformamide (1 mL) was stirred at room temperature for 72 h. Ethyl acetate was added and the mixture washed with water and brine, then concentrated. The residue was purified by silica gel chromatography eluting with 1:3 ethyl acetate:hexanes and the fractions containing product were combined and concentrated. The residue was taken up in a mixture of tetrahydrofuran (0.5 mL), ethyl alcohol (1.5 mL), and water (0.5 mL), then sodium hydroxide (15 mg, 0.35 mmol) was added. The solution was stirred at 50° C. for 2 h, then concentrated to ⅓ volume and added dropwise to hydrochloric acid (0.5 M aq, 5 mL). The mixture was extracted with ethyl acetate twice and the combined extracts were washed with water and brine, then dried over sodium sulfate and concentrated to give 14 mg (14%) of 6-[4-({[3-(2,6-dichlorophenyl)-5-(1-methylethyl)-4-isoxazolyl]methyl}oxy)phenyl]-1H-indazole-3-carboxylic acid as a tan foam. ¹H NMR (400 MHz, d₆-DMSO): δ 12.32 (s, 1H), 9.61 (s, 1H), 7.92 (d, J=9 Hz, 1H), 7.39-7.28 (m, 3H), 7.25-7.18 (m, 3H), 7.18-7.09 (m, 1H), 6.86 (d, J=9 Hz, 2H), 5.50 (s, 2H), 3.69 (septet, J=7 Hz, 1H), 1.38 (d, J=7 Hz, 6H). ESI-LCMS m/z 522 (M+H)⁺.

### Example 9

### 3-[4-({[3-(2,6-Dichlorophenyl)-5-(1-methylethyl)-4-isoxazolyl]methyl}oxy)phenyl]-1-benzothiophene-5-carboxylic Acid

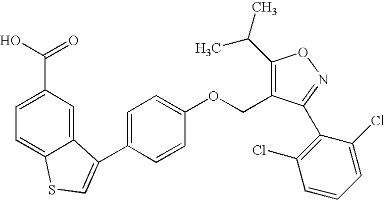

### 9a) Methyl 3-bromo-1-benzothiophene-5-carboxylate

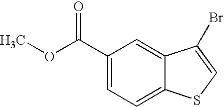

Bromine (48 μL, 0.93 mmol) was added to 1-benzothiophene-5-carboxylic acid (150 mg, 0.84 mmol) in acetic acid (4 mL). The solution was then stirred at room temperature for 4 h. Additional bromine (48 μL, 0.93 mmol) was added and the solution stirred at room temperature for 16 h. The solution was then poured into water (30 mL) with vigorous stirring and the resulting solids were collected by suction filtration, washed with water and dried. The residue was taken up in methanol (5 mL), and thionyl chloride (265 μL, 3.64 mmol) was added. The mixture was heated at reflux for 1 h, then concentrated to give 218 mg (95%) of methyl 3-bromo-1-benzothiophene-5-carboxylate as yellow oil that solidified upon standing. ¹H NMR (400 MHz, CDCl₃): δ 8.52 (s, 1H), 8.07 (d, J=8 Hz, 1H), 7.90 (d, J=8 Hz, 1H), 7.51 (s, 1H), 3.98 (d, 3H).

### 9b) Methyl 3-(4-hydroxyphenyl)-1-benzothiophene-5-carboxylate

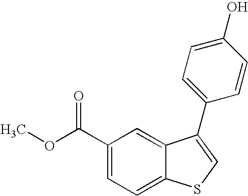

A solution of (4-hydroxyphenyl)boronic acid (102 mg, 0.74 mmol) in ethyl alcohol (0.25 mL) was added to a mixture of methyl 3-bromo-1-benzothiophene-5-carboxylate (100 mg, 0.37 mmol), tetrakis(triphenylphosphine)-palladium (0) (21 mg, 0.02 mmol) and sodium carbonate (2.0 M aq, 460 μL, 0.92 mmol) in 1,2-dimethoxyethane (2 mL.) The mixture was stirred at 90° C. for 2.5 h. Ethyl acetate was added, and the mixture washed with water and brine, then concentrated. The residue was purified by silica gel chromatography eluting with 1:1 ethyl acetate:hexanes to give 59 mg (56%) of methyl 3-(4-hydroxyphenyl)-1-benzothiophene-5-carboxylate as a white solid. ¹H NMR (400 MHz, CDCl₃): δ 8.55 (s, 1H), 8.03 (d, J=9 Hz, 1H), 7.94 (d, J=9 Hz, 1H), 7.46 (d, J=8 Hz, 2H), 7.39 (s, 1H), 6.98 (d, J=8 Hz, 2H), 5.02 (s, 1H), 3.93 (s, 3H).

### 9c) 3-[4-({[3-(2,6-Dichlorophenyl)-5-(1-methylethyl)-4-isoxazolyl]methyl}oxy)phenyl]-1-benzothiophene-5-carboxylic Acid

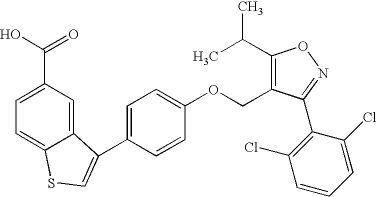

A solution of methyl 3-(4-hydroxyphenyl)-1-benzothiophene-5-carboxylate (58 mg, 0.20 mmol), 4-(chloromethyl)-3-(2,6-dichlorophenyl)-5-(1-methylethyl)isoxazole (125 mg, 0.41 mmol), and potassium carbonate (85 mg, 0.61 mmol) in N,N-dimethylformamide (1.5 mL) was stirred at 60° C. for 16 h. Ethyl acetate was added and the mixture washed with water and brine, then concentrated. The residue was purified by silica gel chromatography eluting with 1:3 ethyl acetate:hexanes and the fractions containing product were combined and concentrated. The residue was taken up in a mixture of tetrahydrofuran (1 mL), ethyl alcohol (3 mL), and water (1 mL). Sodium hydroxide (40 mg, 1.00 mmol) was added. The solution was stirred at room temperature for 72 h, then concentrated to ½ volume and added dropwise to hydrochloric acid (1.0 M aq, 5 mL). The resulting solids were collected by suction filtration, washed with water, then dried to give 80 mg (73%) of 3-[4-({[3-(2,6-dichlorophenyl)-5-(1-methylethyl)-4-isoxazolyl]methyl}oxy)phenyl]-1-benzothiophene-5-carboxylic acid as a white solid. ¹H NMR (400 MHz, d₆-DMSO): δ 8.34 (s, 1H), 8.05 (d, J=8 Hz, 1H), 7.92 (d, J=8 Hz, 1H), 7.73 (s, 1H), 7.63 (d, J=9 Hz, 2H), 7.55-7.51 (m, 1H), 7.42 (d, J=9 Hz, 2H), 6.93 (d, J=9 Hz, 2H), 4.88 (d, 2H), 3.46 (septet, J=7 Hz, 1H), 1.33 (d, J=7 Hz, 6H). ESI-LCMS m/z 538 (M+H)⁺.

### Example 10

### 5-[4-({[3-(2,6-Dichlorophenyl)-5-(1-methylethyl)-4-isoxazolyl]methyl}oxy)phenyl]thieno[3,2-b]pyridine-2-carboxylic Acid

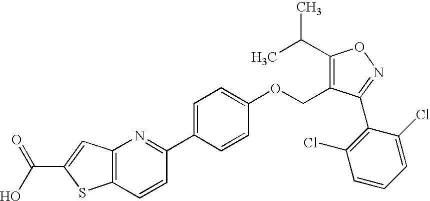

### 10a) 1-(3-Thienyl)ethanone oxime

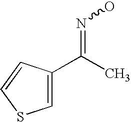

A solution of 3-acetyl thiophene (2.0 g, 15.8 mmol), hydroxylamine hydrochloride (2.06 g, 31.7 mmol), and sodium acetate (4.31 g, 31.7 mmol) in ethyl alcohol (40 mL) was stirred at 70° C. for 24 h. The solution was then poured into water (250 mL) with stirring. The resulting solids were collected by suction filtration, washed with water and dried to give 1.45 g (65%) of 1-(3-thienyl)ethanone oxime as a white solid. ¹H NMR (400 MHz, CDCl₃): δ 7.49 (d, J=3 Hz, 1H), 7.43 (d, J=5 Hz, 1H), 7.31 (dd, J=5, 3 Hz, 1H), 2.38 (s, 3H).

### 10b) 5-Chlorothieno[3,2-b]pyridine

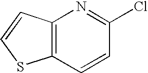

A solution of 1-(3-thienyl)ethanone oxime (250 mg, 1.77 mmol) in polyphosphoric acid (1.5 mL) was stirred at 100° C. for 30 min. The solution was poured into saturated sodium carbonate (aq), then extracted twice with ethyl acetate. The combined extracts were washed with water and brine, then concentrated. The residue was purified by silica gel chromatography eluting with 3:1 ethyl acetate:hexanes and the fractions containing product were combined and concentrated. The residue was taken up in 1,2-dichloroethane (1.5 mL) and then added to a solution of phosphorus oxychloride (225 μL, 2.24 mmol) and N,N-dimethylformamide (63 μL, 0.81 mmol) in 1,2-dichloroethane (0.5 mL) at 0° C. The resulting solution was stirred at room temperature for 15 min, then heated at reflux for 16 h. The solution was then poured in to water (10 mL) containing sodium acetate (550 mg, 4.05 mmol) and the mixture stirred at 100° C. for 20 min. After cooling, water (25 mL) was added and the mixture extracted with ethyl acetate twice. The combined extracts were washed with saturated sodium bicarbonate (aq) and brine, then dried over sodium sulfate and concentrated to give 115 mg (38%) of 5-chlorothieno[3,2-b]pyridine as a pale yellow oil which solidified upon standing. ¹H NMR (400 MHz, CDCl₃): δ 8.12 (d, J=9 Hz, 1H), 7.80 (d, J=5 Hz, 1H), 7.50 (d, J=5 Hz, 1H), 7.28 (d, J=9 Hz, 1H). ESI-LCMS m/z 170 (M+H)⁺.

### 10c) Ethyl 5-{4-[(phenylmethyl)oxy]phenyl}thieno[3,2-b]pyridine-2-carboxylate

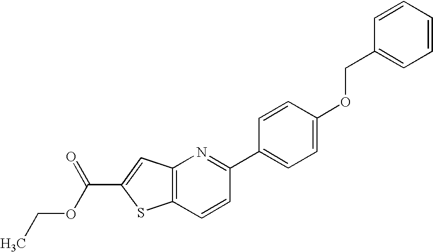

A mixture of 5-chlorothieno[3,2-b]pyridine (115 mg, 0.68 mmol), {4-[(phenylmethyl)oxy]phenyl}boronic acid (230 mg. 1.02 mmol), tetrakis(triphenylphosphine)-palladium (0) (78 mg, 0.07 mmol), and sodium carbonate (2.0 M aq, 1.20 mL, 2.37 mmol) in 1,2-dimethoxyethane (3.5 mL) was stirred at 70° C. for 30 min. Ethyl acetate was added and the mixture filtered through a pad of Celite® and silica gel. The filtrate was washed with water and brine, then concentrated. The residue was purified by silica gel chromatography eluting with 1:1 ethyl acetate:hexanes to give 156 mg of a tan solid. To a solution of the tan solid (78 mg, 0.40 mmol) in tetrahydrofuran (1.5 mL) was added n-butyl lithium (2.5 M in hexanes, 170 μL, 0.43 mmol) at −78° C. The solution was stirred at −78° C. for 1.5 h. Ethyl chloroformate (70 μL, 0.74 mmol) in tetrahydrofuran (0.25 mL) was then added, and the solution was allowed to warm to room temperature over 1 h. Ethyl acetate was added and the solution washed with water and brine, then concentrated. The residue was purified by silica gel chromatography eluting with 1:1 ethyl acetate:hexanes to give 50 mg (26%) of ethyl 5-{4-[(phenylmethyl)oxy]phenyl}thieno[3,2-b]pyridine-2-carboxylate as a beige solid. ¹H NMR (400 MHz, d₆-DMSO): δ 8.57 (d, J=9 Hz, 1H), 8.17-8.12 (m, 2H), 8.05 (d, J=9 Hz, 1H), 7.46 (d, J=8 Hz, 2H), 7.41-7.34 (m, 2H), 7.34-7.32 (m, 1H), 7.15-7.11 (m, 2H), 7.01 (s, 1H), 5.17 (s, 2H), 4.36 (q, J=7 Hz, 2H), 1.34 (t, J=7 Hz, 3H). ESI-LCMS m/z 390 (M+H)⁺.

### 10d) 5-[4-({[3-(2,6-Dichlorophenyl)-5-(1-methylethyl)-4-isoxazolyl]methyl}oxy)phenyl]thieno[3,2-b]pyridine-2-carboxylic Acid

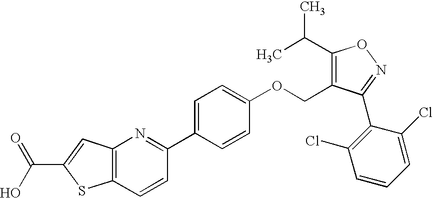

Boron trichloride (1.0 M in dichloromethane, 190 μL, 0.19 mmol) was added to ethyl 5-{4-[(phenylmethyl)oxy]phenyl}thieno[3,2-b]pyridine-2-carboxylate (49 mg, 0.13 mmol) in 1.0 mL dichloromethane at −20° C., then stirred at −20° C. for 5 min. The reaction was quenched with methanol, then concentrated. The residue was taken up in ethyl acetate then washed with water and brine, then concentrated. The residue was taken up in N,N-dimethylformamide (1 mL), then 4-(chloromethyl)-3-(2,6-dichlorophenyl)-5-(1-methylethyl)isoxazole (77 mg, 0.25 mmol) and potassium carbonate (52 mg, 0.38 mmol) were added and the mixture stirred at 60° C. for 16 h. Ethyl acetate was added and the mixture washed with water and brine, then concentrated. The residue was purified by silica gel chromatography eluting with 3:2 ethyl acetate:hexanes and the fractions containing product were combined and concentrated. The residue was taken up in a mixture of tetrahydrofuran (0.5 mL), ethyl alcohol (1 mL), and water (0.25 mL), then sodium hydroxide (10 mg, 0.23 mmol) was added. The solution was stirred at 60° C. for 5 h, then concentrated to ⅓ volume and added dropwise to hydrochloric acid (1.0 M aq, 4 mL). The resulting solids were collected by suction filtration, washed with water, then dried to give 9 mg (13%) of 5-[4-({[3-(2,6-dichlorophenyl)-5-(1-methylethyl)-4-isoxazolyl]methyl}oxy)phenyl]thieno[3,2-b]pyridine-2-carboxylic acid as an pale yellow solid. ¹H NMR (400 MHz, CDCl₃): δ 8.61 (s, 1H), 8.39 (s, 1H), 8.04 (d, J=8 Hz, 2H), 7.82 (d, J=9 Hz, 1H), 7.41 (d, J=8 Hz, 2H), 7.34-7.30 (m, 1H), 6.95 (d, J=8 Hz, 2H), 4.81 (s, 2H), 3.33 (septet, J=7 Hz, 1H), 1.44 (d, J=7 Hz, 6H). ESI-LCMS m/z 539 (M+H)⁺.

### Example 11

### 6-[4-({[3-(2,6-Dichlorophenyl)-5-(1-methylethyl)-4-isoxazolyl]methyl}oxy)phenyl]-1-benzothiophene-3-carboxylic Acid

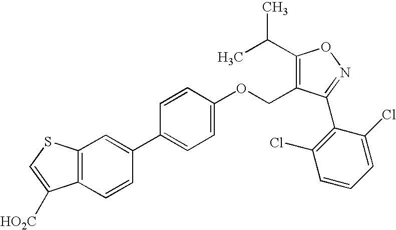

### 11a) 1-{[2,2-Bis(ethyloxy)ethyl]thio}-3-(methyloxy)benzene

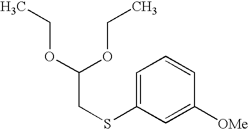

1-{[2,2-Bis(ethyloxy)ethyl]thio}-3-(methyloxy)benzene was prepared according to the general procedure described by S. L. Graham et. al. (J. Med. Chem. (1989), 32(12), 2548-2554) by employing bromoacetaldehyde diethyl acetal (11 mL, 73.1 mmol), 3-methoxybenzenethiol (10 mL, 80.6 mmol), potassium carbonate (11.2 g, 81 mmol) and acetone (100 mL) to give 18.82 g of 1-{[2,2-bis(ethyloxy)ethyl]thio}-3-(methyloxy)benzene as a yellow liquid. The crude product was used without further purification.

### 11b) 6-(Methyloxy)-1-benzothiophene

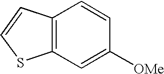

6-(Methyloxy)-1-benzothiophene was prepared according to the general procedure described by S. L. Graham et. al. (J. Med. Chem. (1989), 32(12), 2548-2554) with modification and purified as described by K. Takeuchi et. al. (Bioorg. Med. Chem. Lett. (1999), 9, 759-764). To a stirred solution of boron trifluoride diethyl etherate (9.7 mL, 76.8 mmol) in dichloromethane (1000 mL) was added, very slowly, dropwise, a solution of 1-{[2,2-bis(ethyloxy)ethyl]thio}-3-(methyloxy)benzene (18.8 g) in dichloromethane (150 mL) at room temperature under a nitrogen atmosphere. The reaction mixture was stirred for 30 min. To the stirred reaction mixture was slowly added a saturated aqueous solution of sodium bicarbonate. The reaction mixture was stirred at room temperature for three days. To the reaction mixture was slowly added an additional 500 mL of saturated aqueous sodium bicarbonate and the reaction mixture was stirred for 1 hour. The organic phase was separated, dried over magnesium sulfate, filtered, and the filtrate was concentrated to give the crude product as a dark brown-orange liquid. The crude product was partially purified by flash chromatography over silica gel with hexanes:ethyl acetate (95:5) to give 8.3 g of a ˜3:1 mixture of 6-(methyloxy)-1-benzothiophene and 4-(methyloxy)-1-benzothiophene, respectively. Purification of the 3:1 mixture by flash chromatography over silica gel with a hexanes:ethyl acetate (100:0 to 95:5) gradient failed to purify the desired 6-isomer. Purification of the impure product by flash chromatography over silica gel with hexanes as eluant gave 4.86 g (40% over two steps) of 6-(methyloxy)-1-benzothiophene as a colorless liquid. ¹H NMR (CDCl₃, 400 MHz): δ 7.69 (d, J=9 Hz, 1H), 7.35 (d, J=2 Hz, 1H), 7.25 (m, 2H), 7.00 (dd, J=9, 2 Hz, 1H), 3.87 (s, 3H).

### 11c) 6-(Methyloxy)-1-benzothiophene-3-carboxylic Acid

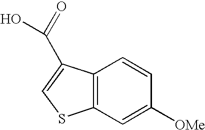

Finely ground aluminum chloride (16.67 g, 125 mmol) was suspended in dichloromethane (150 mL) under a nitrogen atmosphere. The mixture was cooled to −75° C. and a solution of trichloroacetyl chloride (22.73 g, 125 mmol) in dichloromethane (80 mL) was added dropwise over ˜30 min. The mixture was warmed to −40° C. and stirred at that temperature for an additional 45 min. To the reaction mixture was added dropwise over a one hour period a solution of 6-(methyloxy)-1-benzothiophene (20.53 g, material purchased from contract synthesis, 125 mmol) in dichloromethane (80 mL) over a one hour period. The reaction mixture was allowed to warm to 0° C. and stirred at this temperature for 30 min. The reaction mixture was quenched with 1 N hydrochloric acid. To the quenched reaction mixture was added dichloromethane. The organic phase was separated, washed with water followed by saturated aqueous sodium bicarbonate. The organic phase was dried, and the solvent was removed to obtain the crude 2,2,2-trichloro-1-[6-(methyloxy)-1-benzothien-3-yl]ethanone as a complex mixture of compounds. The crude product was applied to a silica gel column and eluted with hexanes:ethyl acetate (95:5) to give ˜10 g of starting material (i.e. 6-(methyloxy)-1-benzothiophene) as well as a mixture of the other components. The recovered starting material was resubmitted to the reaction conditions as described above to provide additional crude 2,2,2-trichloro-1-[6-(methyloxy)-1-benzothien-3-yl]ethanone. The crude 2,2,2-trichloro-1-[6-(methyloxy)-1-benzothien-3-yl]ethanone obtained from each reaction was combined and dissolved in cold tetrahydrofuran (200 mL). To the cold solution was added a chilled 10% aqueous potassium hydroxide solution (200 mL) and the reaction mixture was stirred at room temperature overnight. The volatiles were removed in vacuo and the residue was partitioned between ethyl acetate and potassium hydroxide solution. The organic phase was separated and the pH of the aqueous phase was adjusted to pH˜3 with 1 N hydrochloric acid. The acidic aqueous phase was extracted two times with ethyl acetate. The combined extracts were washed with brine, dried, filtered, and the filtrate was concentrated to give the crude product. The crude product was purified over silica gel with dichloromethane:methanol (97:3) to yield a solid. The solid was ground under cold diethyl ether and the mixture was filtered to give 0.82 g of 6-(methyloxy)-1-benzothiophene-3-carboxylic acid as an off-white solid. ¹H NMR (d₆-DMSO, 400 MHz): δ 12.84 (br s, 1H), 8.39 (s, 1H), 8.32 (d, J=9 Hz, 1H), 7.62 (d, J=2 Hz, 1H), 7.09 (dd, J=2, 9 Hz, 1H), 3.80 (s, 3H). ES-LCMS m/z 207 (M−H)⁻.

### 11d) Methyl 6-(methyloxy)-1-benzothiophene-3-carboxylate

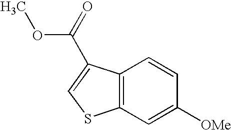

To a mixture of 6-(methyloxy)-1-benzothiophene-3-carboxylic acid (0.341 g, 1.63 mmol) in methanol (15 mL) was slowly added thionyl chloride (0.32 mL, 4.38 mmol) dropwise with stirring at room temperature under a nitrogen atmosphere. The reaction mixture was heated at reflux for 4 hours. The solvent was removed in vacuo. To the solid was added toluene and the solvent was removed in vacuo. The addition of toluene and removal of solvent in vacuo was repeated twice more to give the crude methyl 6-(methyloxy)-1-benzothiophene-3-carboxylate as a brown solid (0.375 g). The compound was used without further purification. ¹H NMR (400 MHz, CDCl₃): δ 8.44 (d, J=9 Hz, 1H), 8.19 (s, 1H), 7.31 (d, J=2 Hz, 1H), 7.10 (dd, J=9, 2 Hz, 1H), 3.93 (s, 3H), 3.88 (s, 3H). ESI-LCMS: m/z 223 (M+H)⁺.

### 11e) Methyl 6-hydroxy-1-benzothiophene-3-carboxylate

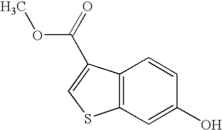

To an ice-water cooled solution of methyl 6-(methyloxy)-1-benzothiophene-3-carboxylate (0.366 g, 1.6 mmol) in dichloromethane (12 mL) was slowly added boron tribromide (1 M in dichloromethane) (7 mL, 7 mmol) with stirring under a nitrogen atmosphere. The reaction mixture was stirred with cooling for 1 hour. The reaction mixture was poured onto ice and the quenched mixture was partitioned between water and dichloromethane. The organic phase was separated and the aqueous phase was extracted with dichloromethane. The organic extracts were combined, washed with brine, dried over magnesium sulfate, filtered, and the filtrate was concentrated to give the crude product as a solid. The crude product was purified by flash chromatography over silica gel with a hexanes:ethyl acetate gradient (100:0 to 50:50) to give 0.174 g of methyl 6-hydroxy-1-benzothiophene-3-carboxylate as a white solid (50% over two steps). ¹H NMR (400 MHz, CDCl₃): δ 8.44 (d, J=9 Hz, 1H), 8.19 (s, 1H), 7.29 (d, J=2 Hz, 1H), 7.02 (dd, J=9, 2 Hz, 1H), 4.83 (br s, 1H), 3.93 (s, 3H). ESI-LCMS m/z 207 (M−H)⁻.

### 11f) Methyl 6-{[(trifluoromethyl)sulfonyl]oxy}-1-benzothiophene-3-carboxylate

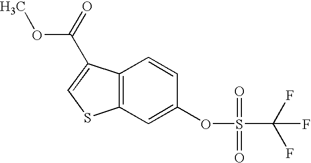

To an ice-water cooled suspension of methyl 6-hydroxy-1-benzothiophene-3-carboxylate (0.164 g, 0.787 mmol) in dichloromethane (7 mL) was slowly added pyridine (0.38 mL, 4.7 mmol) with stirring under a nitrogen atmosphere. The reaction mixture was allowed to stir for 5 min and trifluoromethanesulfonic anhydride (0.18 mL, 1.06 mmol) was slowly added dropwise. The reaction mixture was stirred with cooling for 3 hours. To the reaction mixture was added trifluoromethanesulfonic anhydride (0.05 mL, 0.28 mmol). The reaction mixture was stirred with cooling for 1 hour. The reaction mixture was partitioned between 1 N hydrochloric acid and diethyl ether. The organic phase was separated, dried over magnesium sulfate, filtered, and the filtrate was concentrated to give 0.27 g (100%) of methyl 6-{[(trifluoromethyl)sulfonyl]oxy}-1-benzothiophene-3-carboxylate as a solid. The product was stored in the freezer. ¹H NMR (400 MHz, CDCl₃): δ 8.68 (d, J=9 Hz, 1H), 8.46 (s, 1H), 7.81 (d, J=2 Hz, 1H) 7.40 (dd, J=9, 2 Hz, 1H), 3.96 (s, 3H).

### 11g) Methyl 6-(4-hydroxyphenyl)-1-benzothiophene-3-carboxylate

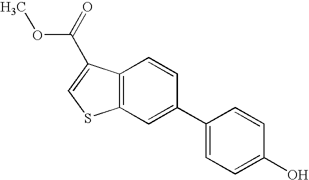

Methyl 6-{[(trifluoromethyl)sulfonyl]oxy}-1-benzothiophene-3-carboxylate (0.27 g, 0.79 mmol), 4-hydroxybenzene boronic acid (0.18 g, 1.3 mmol), tetrakistriphenylphosphine palladium (0) (0.052 g, 0.045 mmol), sodium carbonate (2 M) (4 mL, 8 mmol) and 1,2-dimethoxyethane (10 mL) were combined and the reaction mixture was heated at 80° C. with stirring under a nitrogen atmosphere for 3 hours. The reaction mixture was allowed to cool at room temperature and partitioned between water and ethyl acetate. The organic phase was separated and the aqueous phase was extracted with ethyl acetate. The organic extracts were combined, washed with brine, dried over magnesium sulfate, filtered, and the filtrate was concentrated to give an oil. The crude product was purified by flash chromatography over silica gel with a hexanes:ethyl acetate gradient (100:0 to 60:40) to give 0.17 g (76%) of methyl 6-(4-hydroxyphenyl)-1-benzothiophene-3-carboxylate as a solid. ¹H NMR (400 MHz, CDCl₃): δ 8.59 (d, J=9 Hz, 1H), 8.35 (s, 1H), 8.00 (d, J=2 Hz, 1H), 7.67 (dd, J=9, 2 Hz, 1H), 7.55 (m, 2H), 6.93 (m, 2H), 4.76 (br s, 1H), 3.96 (s, 3H). ES LCMS m/z 283 (M−H)⁻.

### 11h) Methyl 6-[4-({[3-(2,6-dichlorophenyl)-5-(1-methylethyl)-4-isoxazolyl]methyl}oxy)phenyl]-1-benzothiophene-3-carboxylate

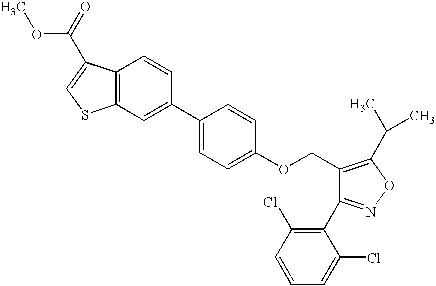

Methyl 6-(4-hydroxyphenyl)-1-benzothiophene-3-carboxylate (0.085 g, 0.30 mmol), [3-(2,6-dichlorophenyl)-5-(1-methylethyl)-4-isoxazolyl]methanol (prepared using Maloney, P. R.; et al; J. Med. Chem.; 43; 16; 2000; 2971-2974) (0.091 g, 0.32 mmol), triphenylphosphine (Polymer-bound on polystyrene, 1 mmol/g) (0.34 g, 0.34 mmol), and dichloromethane (10 mL) were combined in a round-bottom flask and diisopropylazodicarboxylate (0.065 mL, 0.33 mmol) was slowly added dropwise to the reaction mixture with stirring at room temperature under a nitrogen atmosphere. The reaction mixture was stirred overnight, filtered, and the resin was washed with dichloromethane several times. The filtrate was concentrated to give an oil. The crude product was purified by flash chromatography over silica gel with a hexanes:ethyl acetate gradient (100:0 to 75:25) to give 0.090 g (54%) of methyl 6-[4-({[3-(2,6-dichlorophenyl)-5-(1-methylethyl)-4-isoxazolyl]methyl}oxy)phenyl]-1-benzothiophene-3-carboxylate as a colorless oil. ¹H NMR (400 MHz, CDCl₃): δ 8.59 (d, J=9 Hz, 1H), 8.35 (s, 1H), 7.98 (d, J=1 Hz, 1H), 7.64 (dd, J=9, 2 Hz, 1H), 7.52 (m, 2H), 7.42, (d, J=1 Hz, 1H), 7.40 (s, 1H), 7.32 (m, 1H), 6.88 (m, 2H), 4.78 (s, 2H), 3.96 (s, 3H), 3.35 (septet, J=7 Hz, 1H), 1.44 (d, J=7 Hz, 6H). ES-LCMS m/z 552 (M+H)⁺.

### 11i) 6-[4-({[3-(2,6-Dichlorophenyl)-5-(1-methylethyl)-4-isoxazolyl]methyl}oxy)phenyl]-1-benzothiophene-3-carboxylic Acid

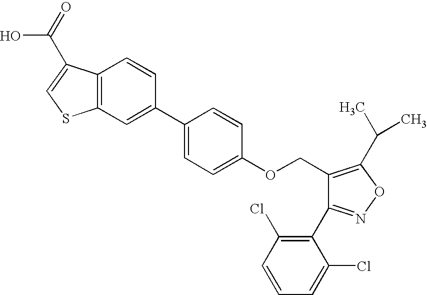

To a stirred solution of methyl 6-[4-({[3-(2,6-dichlorophenyl)-5-(1-methylethyl)-4-isoxazolyl]methyl}oxy)phenyl]-1-benzothiophene-3-carboxylate (0.09 g, 0.16 mmol) in 1,4-dioxane (3 mL) was added 1 N lithium hydroxide (0.32 mL, 0.32 mmol) at room temperature under a nitrogen atmosphere. After 4 hours, methanol (0.75 mL) was added to the reaction mixture and stirring was continued for 23.5 hours. The solvent was removed in vacuo and the crude product was partitioned between water (3 mL), saturated sodium hydrogen sulfate (0.1 mL), and ethyl acetate (10 mL). The organic phase was separated, washed with water (2 mL) followed by brine (2 mL), dried over magnesium sulfate, filtered, and the filtrate was concentrated to give an oil. The oil was dissolved in dichloromethane and the solution was concentrated. The product was once again dissolved in dichloromethane and the solvent was removed in vacuo. The product was dried under high vacuum at 60° C.-75° C. to give 0.059 g (69%) of 6-[4-({[3-(2,6-dichlorophenyl)-5-(1-methylethyl)-4-isoxazolyl]methyl}oxy)phenyl]-1-benzothiophene-3-carboxylic acid as a white solid. ¹H NMR (400 MHz, d₆-DMSO): δ 12.90 (br s, 1H), 8.58 (s, 1H), 8.47 (d, J=9 Hz, 1H), 8.28 (d, J=1 Hz, 1H), 7.71 (dd, J=9, 2 Hz, 1H), 7.62 (m, 4H), 7.53 (m, 1H), 6.88 (d, J=9 Hz, 2H), 4.85 (s, 2H), 3.45 (septet, J=7 Hz, 1H), 1.32 (d, J=7 Hz, 6H). AP-LCMS m/z 538 (M+H)⁺.

### Example 12

### 5-[4-({[3-(2,6-Dichlorophenyl)-5-(1-methylethyl)-4-isoxazolyl]methyl}oxy)phenyl]-1-benzothiophene-2-carboxylic Acid

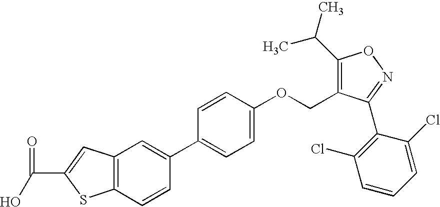

### 12a) 3-(2,6-Dichlorophenyl)-5-(1-methylethyl)-4-({[4-(4,4,5,5-tetramethyl-1,3,2-dioxaborolan-2-yl)phenyl]oxy}methyl)isoxazole

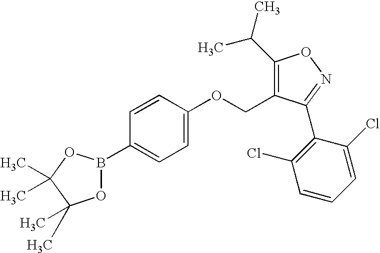

4-(4,4,5,5-Tetramethyl-1,3,2-dioxaborolan-2yl)phenol (1.0 g, 4.5 mmol), [3-(2,6-dichlorophenyl)-5-(1-methylethyl)-4-isoxazolyl]methanol (prepared using Maloney, P. R.; et al; J. Med. Chem.; 43; 16; 2000; 2971-2974) (1.3 g, 4.5 mmol), triphenylphosphine (Polymer-bound on polystyrene resin, 3 mmol/g) (1.57 g, 4.7 mmol), and dichloromethane (50 mL) were combined, and the stirred mixture was cooled in an ice-water bath. To the cold mixture was added dropwise diisopropylazodicarboxylate (0.9 mL, 4.57 mmol) under a nitrogen atmosphere. The ice-water bath was removed and the reaction mixture was allowed to stir at room temperature overnight. The reaction mixture was filtered, and the resin was washed with dichloromethane. The filtrate was concentrated to give the crude product as a yellow oil. The crude product was purified by flash chromatography over silica gel with a hexanes:ethyl acetate gradient (100:0 to 70:30) to give 1.2 g (55%) of 3-(2,6-dichlorophenyl)-5-(1-methylethyl)-4-({[4-(4,4,5,5-tetramethyl-1,3,2-dioxaborolan-2-yl)phenyl]oxy}methyl)isoxazole as a white solid. ¹H NMR (CDCl₃, 400 MHz): δ 7.68 (d, J=9 Hz, 2H), 7.39 (m, 2H), 7.30 (m, 1H), 6.76 (d, J=9 Hz, 2H), 4.73 (s, 2H), 3.32 (septet, J=7 Hz, 1H), 1.41 (d, J=7 Hz, 6H), 1.31 (s, 12H). ES-LCMS m/z 488 (M+H)⁺.

### 12b) 5-[4-({[3-(2,6-Dichlorophenyl)-5-(1-methylethyl)-4-isoxazolyl]methyl}oxy)phenyl]-1-benzothiophene-2-carboxylic Acid

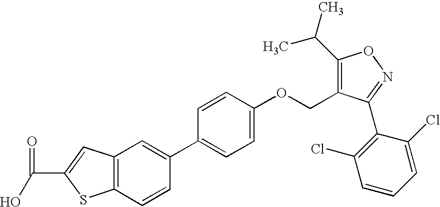

3-(2,6-Dichlorophenyl)-5-(1-methylethyl)-4-({[4-(4,4,5,5-tetramethyl-1,3,2-dioxaborolan-2-yl)phenyl]oxy}methyl)isoxazole (0.178 g, 0.36 mmol), 5-bromo-1-benzothiophene-2-carboxylic acid (0.114 g, 0.44 mmol), tetrakistriphenylphosphine palladium (0) (0.030 g, 0.026 mmol), sodium carbonate (2 M) (0.8 mL, 1.6 mmol), and 1,2-dimethoxyethane (15 mL) were combined and heated at 85° C. with stirring under a nitrogen atmosphere for 4 hours. The reaction mixture was allowed to cool at room temperature. To the reaction mixture was added water and the pH of the aqueous mixture was adjusted to 2-3 (litmus paper) with 1 N hydrochloric acid. The acidic aqueous mixture was extracted with ethyl acetate. The organic phase was separated, washed with brine, dried over magnesium sulfate, filtered, and the filtrate was concentrated to give the crude product as a brown-orange oil. The crude product was purified by reverse phase preparative HPLC using a gradient of acetonitrile:water (50:50 to 100:0) with 0.05% trifluoroacetic acid as a modifier to give a white amorphous solid which was dried at 50° C. under high vacuum to give 0.019 g (10%) of 5-[4-({[3-(2,6-Dichlorophenyl)-5-(1-methylethyl)-4-isoxazolyl]methyl}oxy)phenyl]-1-benzothiophene-2-carboxylic acid as a white amorphous solid. ¹H NMR (d₆-DMSO, 400 MHz): δ 13.53 (br s, 1H), 8.21 (s, 1H), 8.13 (s, 1H), 8.08 (d, J=8 Hz, 1H), 7.75 (d, J=8 Hz, 1H), 7.61 (m, 5H), 6.92 (d, J=9 Hz, 2H), 4.81 (s, 2H), 3.49 (septet, J=7 Hz, 1H), 1.36 (d, J=7 Hz, 6H). ES-LCMS m/z 538 (M+H)⁺.

### Example 13

### 6-[6-({[3-(2,6-Dichlorophenyl)-5-(1-methylethyl)-4-isoxazolyl]methyl}oxy)-3-pyridinyl]-1H-indole-3-carboxylic Acid

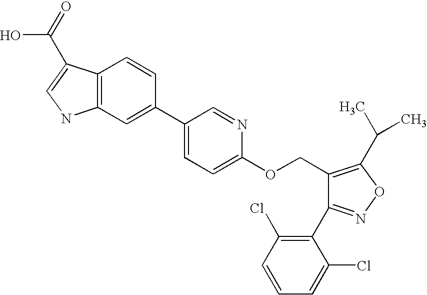

### 13a) 6-Bromo-1H-indole-3-carbaldehyde

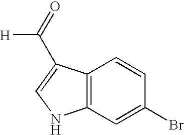

This compound was prepared according to the general procedure described by M. A. Wuonola et. al. (J. Org. Chem., (1994), 59, 6823-6827). To ice-water cooled N,N-dimethylformamide (10 mL) was slowly added phosphorus oxychloride (3.2 mL, 34.6 mmol) with stirring under a nitrogen atmosphere while maintaining the temperature between 0° C. and 8° C. The reaction mixture was stirred at 0° C. for 30 min. To the cold reaction mixture was slowly added a solution of 6-bromoindole (5.5 g, 28.1 mmol) in N,N-dimethylformamide (28 mL) while maintaining the temperature of the reaction mixture between 0° C. and 10° C. The ice-water bath was removed and the reaction mixture was allowed to stir at room temperature for 2 hours. The viscous mixture was poured into ice-water (250 g) and the pH of the cold aqueous mixture was adjusted to ˜7 (litmus paper) with 1 N sodium hydroxide. The mixture was allowed to stand at room temperature overnight. The mixture was filtered to give a pink solid which was washed with water and recrystallized from ethyl alcohol to give 1.6 g (25%) of 6-bromo-1H-indole-3-carbaldehyde as a pale tan solid. ¹H NMR (d₆-DMSO, 400 MHz): δ 12.20 (br s, 1H), 9.91 (s, 1H), 8.31 (d, J=3 Hz, 1H), 8.00 (d, J=9 Hz, 1H), 7.69 (d, J=2 Hz, 1H), 7.34 (dd, J=8, 2 Hz, 1H).

### 13b) Methyl 6-bromo-1H-indole-3-carboxylate

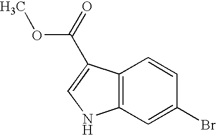

To a stirred solution of 6-bromo-1H-indole-3-carbaldehyde (1.6 g, 7.1 mmol) in methanol (70 mL) was added sodium cyanide (1.7 g, 34.7 mmol) at room temperature. The reaction mixture was stirred for five minutes and then manganese (IV) oxide (7.4 g, 85.1 mmol) was added portionwise over a period of 2.5 hours. The reaction mixture was stirred overnight at room temperature under a nitrogen atmosphere. To the reaction mixture was added dichloromethane (75 mL). The reaction mixture was filtered through a pad of Celite® and the pad was washed with dichloromethane. The cloudy filtrate was concentrated in vacuo and the residue was partitioned between water and ethyl acetate. The organic phase was separated, washed with water, dried over magnesium sulfate, filtered, and the filtrate was concentrated to give the crude product as an off-white solid. The crude product was purified by flash chromatography over silica gel with a hexanes:ethyl acetate gradient (100:0 to 0:100) to give 0.636 g (51% based on recovered starting material) of methyl 6-bromo-1H-indole-3-carboxylate as an off-white solid. ¹H NMR (d₆-DMSO, 400 MHz): δ 12.02 (br s, 1H), 8.09 (s, 1H), 7.90 (d, J=9 Hz, 1H), 7.65 (d, J=2 Hz, 1H), 7.31 (dd, J=9, 2 Hz, 1H), 3.78 (s, 3H). ES-LCMS m/z 252 (M−H)⁻.

### 13c) Methyl 6-(6-fluoro-3-pyridinyl)-1H-indole-3-carboxylate

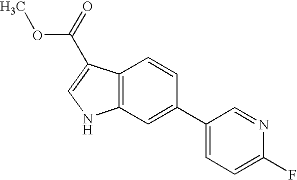

Methyl 6-bromo-1H-indole-3-carboxylate (0.63 g, 2.48 mmol), 2-fluoropyridyl-5-boronic acid (0.435 g, 3.09 mmol), tetrakistriphenylphosphine palladium (0) (0.14 g, 0.012 mmol), 2 M sodium carbonate (5 mL, 10 mmol), and 1,2-dimethoxyethane (20 mL) were combined and heated at reflux with stirring under a nitrogen atmosphere for 15 hours. The reaction mixture was allowed to cool at room temperature and partitioned between water and ethyl acetate. The organic phase was separated, dried over magnesium sulfate, filtered, and the filtrate was concentrated to give the crude product as a yellow solid. The crude product was purified by flash chromatography over silica gel with a dichloromethane:methanol gradient (100:0 to 97:3) to give 0.606 g of methyl 6-(6-fluoro-3-pyridinyl)-1H-indole-3-carboxylate as a yellow solid. ¹H NMR indicates that a minor impurity is present. The compound was used without further purification. ¹H NMR (d₆-DMSO, 400 MHz): δ 12.09 (br s, 1H), 8.54 (d, J=2 Hz, 1H), 8.28 (dt, J=8, 3 Hz, 1H), 8.13 (d, J=3 Hz, 1H), 8.06 (d, J=8 Hz, 1H), 7.73 (s, 1H), 7.51 (dd, J=8, 2 Hz, 1H), 7.27 (dd, J=9, 3 Hz, 1H), 3.80 (s, 3H). ES-LCMS m/z 269 (M−H)⁻.

### 13d) Methyl 6-[6-({[3-(2,6-dichlorophenyl)-5-(1-methylethyl)-4-isoxazolyl]methyl}oxy)-3-pyridinyl]-1H-indole-3-carboxylate

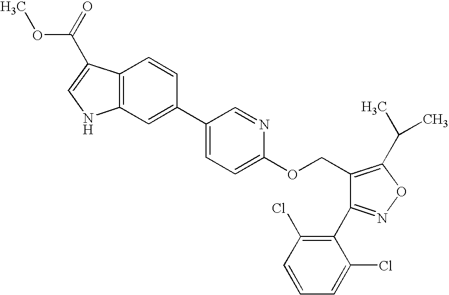

Methyl 6-(6-fluoro-3-pyridinyl)-1H-indole-3-carboxylate (0.606 g), [3-(2,6-dichlorophenyl)-5-(1-methylethyl)-4-isoxazolyl]methanol (prepared using Maloney, P. R.; et al; J. Med. Chem.; 43; 16; 2000; 2971-2974) (0.71 g, 2.48 mmol), and 2-methyl-2-propanol (20 mL) were combined followed by potassium tert-butoxide (0.526 g, 4.69 mmol). The reaction mixture was heated at 80° C. with stirring under a nitrogen atmosphere for 3 hours. The reaction mixture was allowed to stand at room temperature overnight. The reaction mixture was partitioned between water and ethyl acetate. The organic phase was separated and the pH of the aqueous phase was adjusted to ˜5-6 (litmus paper) with 10% citric acid. The slightly acidic aqueous phase was combined with the aforementioned ethyl acetate phase and the mixture was agitated. The organic phase was separated, washed with brine, dried over magnesium sulfate, filtered, and the filtrate was concentrated to give a gold-yellow viscous oil. The crude product was purified by flash chromatography over silica gel with a dichloromethane:methanol gradient (100:0 to 96:4) to give 0.235 g (18% over two steps; i.e. the previous yield was a crude yield and the combined yield between steps of 13c and 13d is 18%) of methyl 6-[6-({[3-(2,6-dichlorophenyl)-5-(1-methylethyl)-4-isoxazolyl]methyl}oxy)-3-pyridinyl]-1H-indole-3-carboxylate as an off-white solid. ¹H NMR (d₆-DMSO, 400 MHz): δ 12.00 (br s, 1H), 8.25 (d, J=2 Hz, 1H), 8.10 (d, J=3 Hz, 1H), 8.02 (d, J=8 Hz, 1H), 7.93 (dd, J=9, 3 Hz, 1H), 7.61 (m, 3H), 7.54 (m, 1H), 7.40 (dd, J=8, 2 Hz, 1H), 6.67 (d, J=9 Hz, 1H), 5.13 (s, 2H), 3.79 (s, 3H), 3.56 (septet, J=7 Hz, 1H), 1.34 (d, J=7 Hz, 6H). AP-LCMS m/z 558 (M+Na)⁺.

### 13e) 6-[6-({[3-(2,6-Dichlorophenyl)-5-(1-methylethyl)-4-isoxazolyl]methyl}oxy)-3-pyridinyl]-1H-indole-3-carboxylic Acid

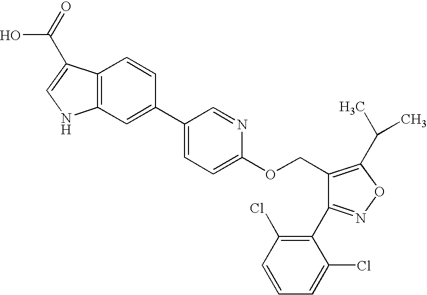

To a round-bottom flask were added methyl 6-[6-({[3-(2,6-dichlorophenyl)-5-(1-methylethyl)-4-isoxazolyl]methyl}oxy)-3-pyridinyl]-1H-indole-3-carboxylate (0.102 g, 0.19 mmol), tetrahydrofuran (4 mL), and methanol (2 mL) followed by 1 N sodium hydroxide (0.40 mL, 0.40 mmol). The reaction mixture was stirred at room temperature under a nitrogen atmosphere for 21 hours. To the reaction mixture was added 1 N sodium hydroxide (0.80 mL, 0.80 mmol). The reaction mixture was heated at 50° C. with stirring under a nitrogen atmosphere. After 2 hours, 1 N sodium hydroxide (0.8 mL, 0.80 mmol) was added to the reaction mixture and heating was continued at 50° C. for 46 hours. The methanol and tetrahydrofuran were removed in vacuo and the aqueous mixture was diluted with water (5 mL). The pH of the aqueous mixture was adjusted to ˜4-5 (litmus paper) with 10% citric acid. To the acidic aqueous mixture was added dichloromethane and the mixture was agitated. The two phases separated minimally upon standing. Brine was added to the mixture to facilitate phase separation. The organic phase was separated, dried over magnesium sulfate, filtered, and the filtrate was concentrated to give a white solid. The crude product was purified by flash chromatography over silica gel with a dichloromethane:methanol gradient (100:0 to 95:5) to give 0.034 g (34%) of 6-[6-({[3-(2,6-dichlorophenyl)-5-(1-methylethyl)-4-isoxazolyl]methyl}oxy)-3-pyridinyl]-1H-indole-3-carboxylic acid as a white solid. ¹H NMR (d₆-DMSO, 400 MHz): δ 11.97 (br s, 1H), 11.87 (br s, 1H), 8.25 (d, J=3 Hz, 1H), 8.02 (m, 2H), 7.93 (dd, J=9, 3 Hz, 1H), 7.60 (m, 3H), 7.53 (m, 1H), 7.37 (d, J=9 Hz, 1H), 6.66 (d, J=9 Hz, 1H), 5.13 (s, 2H), 3.56 (septet, J=7 Hz, 1H), 1.34 (d, J=7 Hz, 6H). ES-LCMS m/z 522 (M+H)⁺.

### Example 14

### 6-[4-({[3-(2,6-Dichlorophenyl)-5-(1-methylethyl)-4-isoxazolyl]methyl}oxy)phenyl]-4-oxo-4H-chromene-2-carboxylic Acid

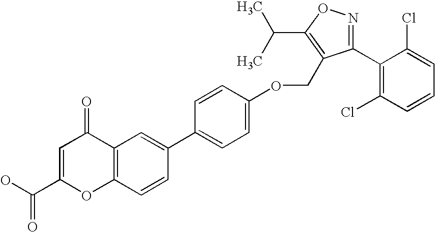

### 14a) Ethyl 6-(4-hydroxyphenyl)-4-oxo-4H-chromene-2-carboxylate

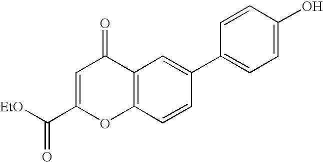

A mixture of ethyl-6-iodo-4-oxo-4H-chromene-2-carboxylate (0.96 g, 2.8 mmol), 4-hydroxyphenyl boronic acid (0.60 g, 4.35 mmol), 1,1′-(bis(diphenylphosphino)ferrocene)dichloropalladium (II) (0.10 g, 0.137 mmol), potassium phosphate (2.4 g, 11.3 mmol), and 1,2-dimethoxyethane (typically 20-40 mL) was heated in a flask at 85° C. with stirring under a nitrogen atmosphere for 37 hours. The reaction mixture was allowed to cool at room temperature and partitioned between water and ethyl acetate. The organic phase was separated, dried over magnesium sulfate, filtered, and the filtrate was concentrated to give a brown solid. The crude product was partially purified by flash chromatography over silica gel with a hexanes:ethyl acetate gradient (100:0 to 0:100) to give a tan solid. The tan solid was purified by flash chromatography over silica gel with a dichloromethane:methanol gradient (100:0 to 99:1) to give 0.117 g (13%) of ethyl 6-(4-hydroxyphenyl)-4-oxo-4H-chromene-2-carboxylate as a gold-yellow solid. ¹H NMR (CDCl₃, 400 MHz): δ 8.34 (d, J=2 Hz, 1H), 7.94 (dd, J=9, 2 Hz, 1H), 7.66 (d, J=9 Hz, 1H), 7.55 (d, J=9 Hz, 2H), 7.14 (s, 1H), 6.94 (d, J=9 Hz, 2H), 6.71 (s, 1H), 4.47 (q, J=7 Hz, 2H), 1.44 (t, J=7 Hz, 3H). AP-LCMS m/z 311 (M+H⁺)⁺.

### 14b) 4-(Chloromethyl)-3-(2,6-dichlorophenyl)-5-(1-methylethyl)isoxazole

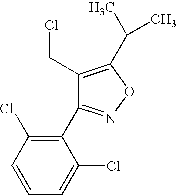

To a stirred solution of [3-(2,6-dichlorophenyl)-5-(1-methylethyl)-4-isoxazolyl]methanol (prepared using Maloney, P. R.; et al; J. Med. Chem.; 43; 16; 2000; 2971-2974) (0.25 g, 0.87 mmol) in dichloromethane (10 mL) was added dropwise thionyl chloride (0.2 mL, 2.74 mmol) at room temperature under a nitrogen atmosphere. The reaction mixture was stirred for 2.5 hours. The reaction mixture was concentrated in vacuo. The crude product was dissolved in dichloromethane and the solution was concentrated in vacuo. This was repeated twice more to give 0.293 g (>100%) of 4-(chloromethyl)-3-(2,6-dichlorophenyl)-5-(1-methylethyl)isoxazole as a yellow oil. The compound was used without further purification. ¹H NMR (d₆-DMSO, 400 MHz): δ 7.64 (m, 2H), 7.58 (m, 1H), 4.47 (s, 2H), 3.45 (septet, J=7 Hz, 1H), 1.31 (d, J=7 Hz, 6H).

### 14c) Ethyl 6-[4-({[3-(2,6-dichlorophenyl)-5-(1-methylethyl)-4-isoxazolyl]methyl}oxy)phenyl]-4-oxo-4H-chromene-2-carboxylate

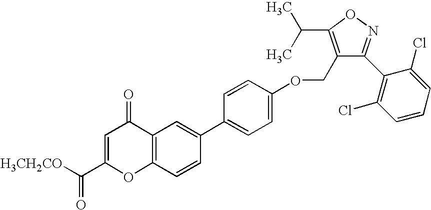

Ethyl 6-(4-hydroxyphenyl)-4-oxo-4H-chromene-2-carboxylate (79% pure according to diode array of AP-LCMS) (0.13 g), cesium carbonate (0.256 g, 0.79 mmol), and N,N-dimethylformamide (6 mL) were combined and the reaction mixture was heated at 65° C. with stirring under a nitrogen atmosphere for 2.5 hours. The oil bath was removed and the reaction mixture was allowed to stand at room temperature for 2 hours. To the reaction mixture was added a solution of 4-(chloromethyl)-3-(2,6-dichlorophenyl)-5-(1-methylethyl)isoxazole (0.158 g, 0.52 mmol) in N,N-dimethylformamide (2.4 mL) and the reaction mixture was heated at 65° C. for 18 hours. The reaction mixture was partitioned between water and ethyl acetate. The organic phase was separated and the aqueous phase was extracted with an additional portion of ethyl acetate. The organic phase was separated. To the turbid aqueous phase was added brine. The aqueous phase was once again extracted with ethyl acetate. The organic extracts were combined, washed with water followed by brine, dried over magnesium sulfate, filtered, and the filtrate was concentrated to give the crude product as a brown oil. The crude product was purified by flash chromatography over silica gel with a hexanes:ethyl acetate gradient (100:0 to 60:40) to give 0.043 g of ethyl 6-[4-({[3-(2,6-dichlorophenyl)-5-(1-methylethyl)-4-isoxazolyl]methyl}oxy)phenyl]-4-oxo-4H-chromene-2-carboxylate as a yellow oil which solidified upon standing. ¹H NMR (CDCl₃, 400 MHz): δ 8.31 (d, J=2 Hz, 1H), 7.90 (dd, J=9, 2 Hz, 1H), 7.65 (d, J=9 Hz, 1H), 7.52 (d, J=9 Hz, 2H), 7.41 (d, J=8 Hz, 2H), 7.32 (m, 1H), 7.13 (s, 1H), 6.86 (d, J=9 Hz, 2H), 4.77 (s, 2H), 4.47 (q, J=7 Hz, 2H), 3.35 (septet, J=7 Hz, 1H), 1.44 (m, 9H). ES-LCMS m/z 578 (M+H⁺)⁺.

### 14d) 6-[4-({[3-(2,6-Dichlorophenyl)-5-(1-methylethyl)-4-isoxazolyl]methyl}oxy)phenyl]-4-oxo-4H-chromene-2-carboxylic Acid

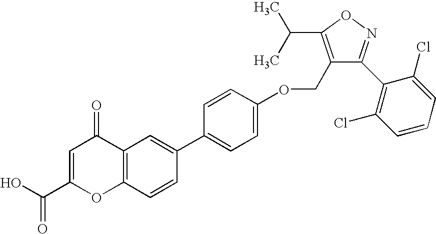

To a solution of ethyl 6-[4-({[3-(2,6-dichlorophenyl)-5-(1-methylethyl)-4-isoxazolyl]methyl}oxy)phenyl]-4-oxo-4H-chromene-2-carboxylate (0.041 g, 0.071 mmol) in tetrahydrofuran (2 mL) and ethyl alcohol (1 mL) was added 2 M sodium bicarbonate (0.37 mL, 0.74 mmol). The turbid reaction mixture was stirred at 70° C. under a nitrogen atmosphere for 4.5 h. The reaction mixture was allowed to cool at room temperature and concentrated in vacuo. Water (5 mL) was added to the crude product and the pH of the aqueous mixture was adjusted to ˜1 (litmus paper) with 1 N hydrochloric acid. The acidic aqueous mixture was extracted with ethyl acetate. The organic extract was washed with water followed by brine, dried over magnesium sulfate, filtered, and the filtrate was allowed to stand overnight at room temperature. The filtrate was concentrated to give the crude product as a yellow solid. To the crude product was added dichloromethane. The yellow solid was filtered and dried at 50° C. under high vacuum to give 0.0089 g (23%) of 6-[4-({[3-(2,6-dichlorophenyl)-5-(1-methylethyl)-4-isoxazolyl]methyl}oxy)phenyl]-4-oxo-4H-chromene-2-carboxylic acid. ¹H NMR (d₆-DMSO, 400 MHz): δ 8.12 (m, 1H), 8.09 (dd, J=9, 2 Hz, 1H), 7.77 (d, J=9 Hz, 1H), 7.61 (m, 4H), 7.53 (dd, J=9, 7 Hz, 1H), 6.89 (m, 3H), 4.85 (s, 2H), 3.45 (septet, J=7 Hz, 1H), 1.32 (d, J=7 Hz, 6H).

### Example 15

### 7-[4-({[3-(2,6-Dichlorophenyl)-5-(1-methylethyl)-4-isoxazolyl]methyl}oxy)phenyl]-2-oxo-2H-chromene-4-carboxylic Acid

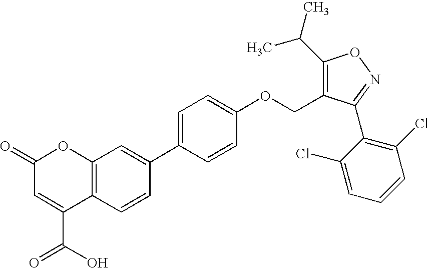

### 15a) Ethyl 2-oxo-7-{[(trifluoromethyl) sulfonyl]oxy}-2H-chromene-4-carboxylate

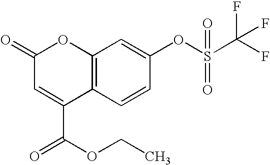

Ethyl 7-hydroxy-2-oxo-2H-chromene-4-carboxylate (1.5 g, 6.40 mmol) and pyridine (3.1 mL, 38.4 mmol) in dichloromethane (20 mL) were cooled to 0° C. Trifluoromethanesulfonic anhydride (1.3 mL, 7.69 mmol) in dichloromethane (10 mL) was slowly added to the reaction mixture. The reaction mixture was stirred at 0° C. for 40 mins. The reaction mixture was then diluted with water followed by diethyl ether. The layers were separated and the ether layer was washed with water several times, followed by brine, then dried over magnesium sulfate, filtered, and concentrated to afford a crude oil. The crude material was purified using hexanes:ethyl acetate (30% ethyl acetate) to afford 2.05 g (87%) of ethyl 2-oxo-7-{[(trifluoromethyl) sulfonyl]oxy}-2H-chromene-4-carboxylate. ¹H NMR (400 MHz, d₆-DMSO): δ 8.31 (d, J=9 Hz, 1H), 7.83 (d, J=3 Hz, 1H), 7.55 (dd, J=9, 3 Hz, 1H), 7.00 (s, 1H), 4.39 (q, J=7 Hz, 2H), 1.33 (t, J=7 Hz, 3H).

### 15b) Ethyl 7-[4-({[3-(2,6-dichlorophenyl)-5-(1-methylethyl)-4-isoxazolyl]methyl}oxy)phenyl]-2-oxo-2H-chromene-4-carboxylate

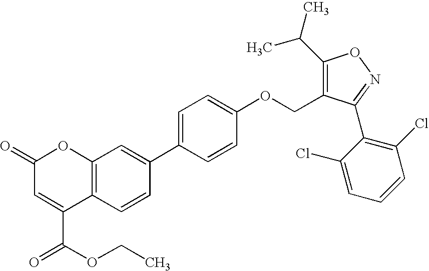

Ethyl 2-oxo-7-{[(trifluoromethyl)sulfonyl]oxy}-2H-chromene-4-carboxylate (0.3 g, 0.819 mmol), 3-(2,6-dichlorophenyl)-5-(1-methylethyl)-4-({[4-(4,4,5,5-tetramethyl-1,3,2-dioxaborolan-2-yl)phenyl]oxy}methyl)isoxazole (0.56 g, 1.15 mmol), (1,1′-bis(diphenylphosphino)ferrocene)dichloropalladium (II) complex (0.033 g, 0.040 mmol) and potassium phosphate (0.695 g, 3.28 mmol) in ethylene glycol dimethyl ether (9 mL) were heated for 2 hours at 85° C. The reaction mixture was cooled to room temperature and diluted with water followed by ethyl acetate. The layers were separated and the ethyl acetate layer was washed several times with water followed by brine, dried over magnesium sulfate, filtered and concentrated to afford dark brown oil. The crude oil was purified using hexanes:ethyl acetate (30% ethyl acetate) to afford 0.16 g (34%) of ethyl 7-[4-({[3-(2,6-dichlorophenyl)-5-(1-methylethyl)-4-isoxazolyl]methyl}oxy)phenyl]-2-oxo-2H-chromene-4-carboxylate as yellow solid. ¹H NMR (400 MHz, d₆-DMSO): δ 8.10 (d, J=9 Hz, 1H), 7.68 (m, 4H), 7.61 (m, 2H), 7.52 (m, 1H), 6.89 (d, J=9 Hz, 2H), 6.84 (s, 1H), 4.86 (s, 2H), 4.40 (q, J=7 Hz, 2H), 3.45 (septet, J=7 Hz, 1H), 1.34 (t, J=7 Hz, 3H), 1.32 (d, J=7 Hz, 6H).

### 15c) 7-[4-({[3-(2,6-Dichlorophenyl)-5-(1-methylethyl)-4-isoxazolyl]methyl}oxy)phenyl]-2-oxo-2H-chromene-4-carboxylic Acid

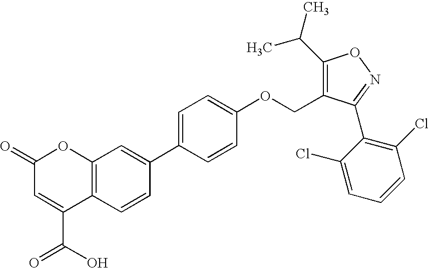

2 N Sodium bicarbonate (0.14 mL) was added to a solution of ethyl 7-[4-({[3-(2,6-dichlorophenyl)-5-(1-methylethyl)-4-isoxazolyl]methyl}oxy)phenyl]-2-oxo-2H-chromene-4-carboxylate (0.16 g, 0.277 mmol), tetrahydrofuran (8 mL) and ethanol (4 mL) and the reaction mixture was stirred at room temperature. After 4 hours the reaction mixture contained only starting material. 2N Sodium bicarbonate (0.70 mL) was added, and the reaction mixture was stirred. After 4 days, there was no reaction, 2 N potassium hydroxide (0.14 mL) was added to the mixture and the reaction was refluxed for 2 hours. The reaction was concentrated to oil then diluted with 1 N hydrochloric acid and ethyl acetate. The layers were separated and the ethyl acetate layer washed with brine, dried over magnesium sulfate, filtered, and concentrated to give an oil. The crude oil was purified using reverse phase HPLC, acetonitrile:water (50 to 100% acetonitrile gradient), to afford 0.07 g (46%) of 7-[4-({[3-(2,6-dichlorophenyl)-5-(1-methylethyl)-4-isoxazolyl]methyl}oxy)phenyl]-2-oxo-2H-chromene-4-carboxylic acid. ¹H NMR (400 MHz, d₆-DMSO): δ 14.34 (br s, 1H), 8.18 (d, J=8 Hz, 1H), 7.67 (m, 4H), 7.61 (m, 2H), 7.52 (m, 1H), 6.89 (d, J=9 Hz, 2H), 6.80 (s, 1H), 4.86 (s, 2H), 3.45 (septet, J=7 Hz, 1H), 1.32 (d, J=7 Hz, 6H). HRMS C₂₉H₂₁Cl₂NO₆ m/z 550.0824 (M+H)⁺_{Cal}; 550.0829 (M+H)⁺_{Obs}.

### Example 16

### 7-[4-({[3-(2,6-Dichlorophenyl)-5-(1-methylethyl)-4-isoxazolyl]methyl}oxy)phenyl]-4-oxo-4H-chromene-2-carboxylic Acid

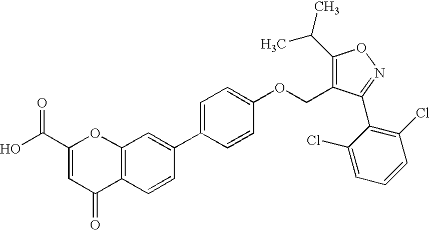

### 16a) Ethyl 4-oxo-7-{[(trifluoromethyl)sulfonyl]oxy}-4H-chromene-2-carboxylate

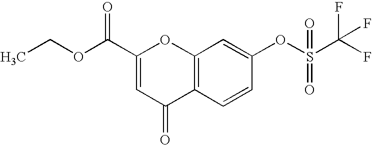

Ethyl 7-hydroxy-4-oxo-4H-chromene-2-carboxylate (1.5 g, 6.40 mmol) and pyridine (3.1 mL, 38.4 mmol) in dichloromethane (20 mL) were brought to 0° C. Trifluoromethanesulfonic anhydride (1.3 mL, 7.69 mmol) in dichloromethane (10 mL) was slowly added to the reaction mixture and the reaction mixture was stirred for 1 hour at room temperature. Then it was diluted with water followed by diethyl ether. The layers were separated. The ether layer was washed with water several times, followed by brine, then dried over magnesium sulfate, filtered, and concentrated to afford a crude oil. The crude material was purified using hexanes:ethyl acetate (30% ethyl acetate) to afford 1.74 g (74%) of ethyl 4-oxo-7-{[(trifluoromethyl)sulfonyl]oxy}-4H-chromene-2-carboxylate. ¹H NMR (400 MHz, d₆-DMSO): δ 8.21 (m, 2H), 7.66 (dd, J=9, 2 Hz, 1H), 7.00 (s, 1H), 4.38 (q, J=7 Hz, 2H), 1.32 (t, J=7 Hz, 3H).

### 16b) Ethyl 7-[4-({[3-(2,6-dichlorophenyl)-5-(1-methylethyl)-4-isoxazolyl]methyl}oxy)phenyl]-4-oxo-4H-chromene-2-carboxylate

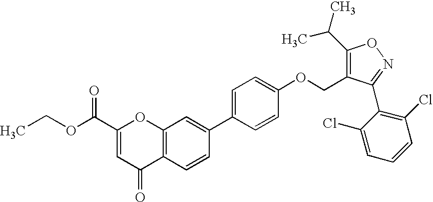

Ethyl 4-oxo-7-{[(trifluoromethyl)sulfonyl]oxy}-4H-chromene-2-carboxylate (0.1 g, 0.273 mmol), 3-(2,6-dichlorophenyl)-5-(1-methylethyl)-4-({[4-(4,4,5,5-tetramethyl-1,3,2-dioxaborolan-2-yl)phenyl]oxy}methyl)isoxazole (0.19 g, 0.382 mmol), (1,1′-bis(diphenylphosphino)ferrocene)dichloropalladium (II) complex (0.011 g, 0.013 mmol) and potassium phosphate (0.23 g, 1.09 mmol) in ethylene glycol dimethyl ether (3 mL) were heated for 4 hours at 85° C. The reaction mixture was cooled to room temperature and diluted with water followed by ethyl acetate. The layers were separated. The ethyl acetate layer was washed several times with water, followed by brine, dried over magnesium sulfate, filtered, and concentrated to afford a dark brown oil. The crude oil was purified using hexanes:ethyl acetate (30% ethyl acetate) to afford 0.11 g (70%) of ethyl 7-[4-({[3-(2,6-dichlorophenyl)-5-(1-methylethyl)-4-isoxazolyl]methyl}oxy)phenyl]-4-oxo-4H-chromene-2-carboxylate. ¹H NMR (400 MHz, d₆-DMSO): δ 8.04 (d, J=8 Hz, 1H), 7.94 (s, 1H), 7.80 (d, J=9 Hz, 1H), 7.75 (d, J=9 Hz, 2H), 7.61 (m, 2H), 7.53 (m, 1H), 6.94 (s, 1H), 6.90 (d, J=9 Hz, 2H), 4.88 (s, 2H), 4.38 (q, J=7 Hz, 2H), 3.46 (septet, J=7 Hz, 1H), 1.34 (m, 9H).

### 16c) 7-[4-({[3-(2,6-Dichlorophenyl)-5-(1-methylethyl)-4-isoxazolyl]methyl}oxy)phenyl]-4-oxo-4H-chromene-2-carboxylic Acid

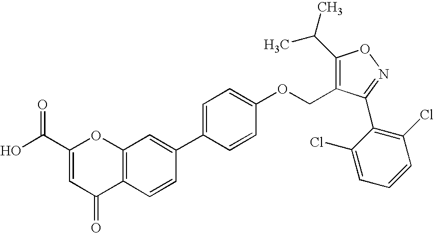

2 N Sodium bicarbonate (0.99 mL) was added to a solution of ethyl 7-[4-({[3-(2,6-dichlorophenyl)-5-(1-methylethyl)-4-isoxazolyl]methyl}oxy)phenyl]-4-oxo-4H-chromene-2-carboxylate (0.11 g, 0.190 mmol) in tetrahydrofuran (5.4 mL) and ethanol (2.7 mL). The reaction mixture was stirred at room temperature for 1 h, then heated at 70° C. for 2 hours. The reaction mixture stirred at room temperature overnight. It was stirred at 70° C. for another hour. 2N sodium carbonate (0.1 mL) was added to the reaction mixture and the reaction mixture was stirred at room temperature. The reaction mixture was concentrated to white solid then diluted with water, followed by 1 N hydrochloric acid. It was then extracted with ethyl acetate. The ethyl acetate layer was washed with brine, dried over magnesium sulfate, filtered, and concentrated to give yellow solid. The yellow solid was dissolved in dichloromethane and dried to afford 79.4 mg (76%) of 7-[4-({[3-(2,6-dichlorophenyl)-5-(1-methylethyl)-4-isoxazolyl]methyl}oxy)phenyl]-4-oxo-4H-chromene-2-carboxylic acid as a yellow powder. ¹H NMR (400 MHz, d₆-DMSO): δ 14.55 (br s, 1H), 8.03 (d, J=9 Hz, 1H), 7.90 (s, 1H), 7.77 (d, J=9 Hz, 1H), 7.73 (d, J=9 Hz, 2H), 7.61 (m, 2H), 7.53 (m, 1H), 6.91 (d, J=9 Hz, 2H), 6.86 (s, 1H), 4.87 (s, 2H), 3.47 (septet, J=7 Hz, 1H), 1.32 (d, J=7 Hz, 6H).

### Example 17

### 6-[4-({[3-(2,6-Dichlorophenyl)-5-(1-methylethyl)-4-isoxazolyl]methyl}oxy)phenyl]-1,2,3,4-tetrahydro-1-naphthalenecarboxylic Acid

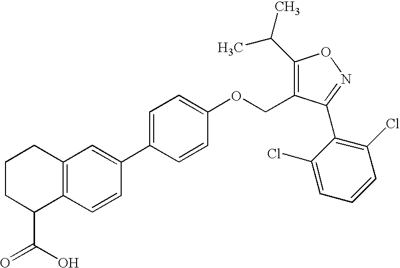

### 17a) Methyl 6-hydroxy-1,2,3,4-tetrahydro-1-naphthalenecarboxylate

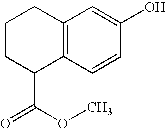

To a round bottom flask containing 6-hydroxy-1,2,3,4-tetrahydro-1-naphthalenecarboxylic acid (0.5 g, 2.60 mmol) in methanol (23 mL) was added thionyl chloride (0.38 mL, 5.2 mmol), and the reaction mixture was refluxed for 3 days. The reaction mixture was cooled to room temperature, then concentrated. The crude material was diluted with water, followed by 5% sodium bicarbonate, and extracted with ethyl acetate. The ethyl acetate layer was washed with brine, dried over magnesium sulfate, filtered, and concentrated to afford 0.5 g (93%) of methyl 6-hydroxy-1,2,3,4-tetrahydro-1-naphthalenecarboxylate. ¹H NMR (400 MHz, d₆-DMSO): δ 9.15 (s, 1H), 6.85 (d, J=8 Hz, 1H), 6.50 (d, J=8 Hz, 1H), 6.45 (s, 1H), 3.67 (t, J=6 Hz, 1H), 3.59 (s, 3H), 2.60 (m, 2H), 1.70-1.98 (m, 3H), 1.55-1.66 (m, 1H).

### 17b) Methyl 6-{[(trifluoromethyl)sulfonyl]oxy}-1,2,3,4-tetrahydro-1-naphthalenecarboxylate

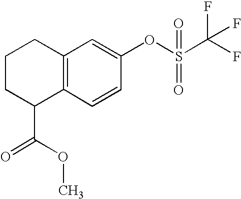

A round bottom flask containing methyl 6-hydroxy-1,2,3,4-tetrahydro-1-naphthalenecarboxylate (0.5 g, 2.42 mmol) and pyridine (1.2 mL, 14.5 mmol) in dichloromethane (7 mL) was cooled to 0° C. Trifluoromethanesulfonic anhydride (0.49 mL, 2.91 mmol) in dichloromethane (3 mL) was slowly added to the reaction mixture and left to stir in an ice-bath. The reaction mixture was stirred at 0° C. for 1.5 hours. It was then diluted with water, followed by diethyl ether. The layers were separated. The ether layer was washed with water several times, followed by brine, then dried over magnesium sulfate, filtered, and concentrated to afford a crude material. The crude material was purified using hexanes:ethyl acetate (30% ethyl acetate) to afford 0.557 g (68%) of methyl 6-{[(trifluoromethyl)sulfonyl]oxy}-1,2,3,4-tetrahydro-1-naphthalenecarboxylate. ¹H NMR (400 MHz, d₆-DMSO): δ 7.28 (d, J=8 Hz, 1H), 7.21 (m, 2H), 3.91 (t, J=6 Hz, 1H), 3.62 (s, 3H), 2.77 (m, 2H), 2.01 (m, 1H), 1.92 (m, 1H). 1.73 (m, 2H)

### 17c) Methyl 6-(4-hydroxyphenyl)-1,2,3,4-tetrahydro-1-naphthalenecarboxylate

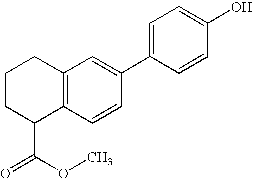

Methyl 6-{[(trifluoromethyl)sulfonyl]oxy}-1,2,3,4-tetrahydro-1-naphthalenecarboxylate (0.3 g, 0.887 mmol), (4-hydroxyphenyl)boronic acid (0.147 g, 1.06 mmol), tetrakis(triphenylphosphine)palladium (0) (0.041 g, 0.035 mmol) and 2 M sodium carbonate (4 mL) in 1,2-dimethoxyethane (5 mL) were heated at 80° C. After 2 hours of heating, the reaction was cooled to room temperature, then diluted with water, followed by ethyl acetate. The layers were separated and the ethyl acetate layer was washed with brine, dried over magnesium sulfate, filtered, and concentrated to afford the crude material. The crude material was purified using hexanes:ethyl acetate (0 to 30% ethyl acetate) to afford 0.165 g (66%) of methyl 6-(4-hydroxyphenyl)-1,2,3,4-tetrahydro-1-naphthalenecarboxylate as a white foam. ¹H NMR (400 MHz, d₆-DMSO): δ 9.48 (s, 1H), 7.42 (d, J=9 Hz, 2H), 7.29 (m, 2H), 7.10 (d, J=8 Hz, 1H), 6.80 (d, J=9 Hz, 2H), 3.83 (t, J=6 Hz, 1H), 3.62 (s, 3H), 2.75 (m, 2H), 1.95 (m, 2H), 1.83 (m, 1H), 1.69 (m, 1H).

### 17d) Methyl 6-[4-({[3-(2,6-dichlorophenyl)-5-(1-methylethyl)-4-isoxazolyl]methyl}oxy)phenyl]-1,2,3,4-tetrahydro-1-naphthalenecarboxylate

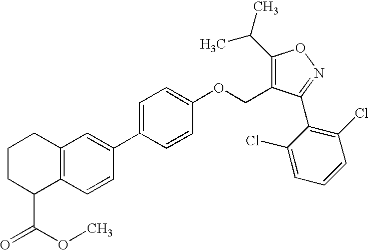

To a stirring solution of methyl 6-(4-hydroxyphenyl)-1,2,3,4-tetrahydro-1-naphthalenecarboxylate (0.165 g, 0.584 mmol), [3-(2,6-dichlorophenyl)-5-(1-methylethyl)-4-isoxazolyl]methanol (0.167 g, 0.584 mmol) and triphenylphosphine (0.153 g, 0.584 mmol) in dichloromethane (9 mL) at 0° C. was added diisopropyl azodicarboxylate (0.115 mL, 0.584 mmol). After stirring at room temperature overnight, the reaction mixture was concentrated to an oil and purified using hexanes:ethyl acetate (25% ethyl acetate) to afford 0.147 g (46%) of methyl 6-[4-({[3-(2,6-dichlorophenyl)-5-(1-methylethyl)-4-isoxazolyl]methyl}oxy)phenyl]-1,2,3,4-tetrahydro-1-naphthalenecarboxylate as an oil. ¹H NMR (400 MHz, d₆-DMSO): δ 7.61 (m, 2H), 7.52 (dd, J=9, 7 Hz, 1H), 7.46 (d, J=9 Hz, 2H), 7.29 (m, 2H), 7.11 (d, J=8 Hz, 1H), 6.82 (d, J=9 Hz, 2H), 4.82 (s, 2H), 3.83 (t, J=6 Hz, 1H), 3.62 (s, 3H), 3.43 (septet, J=7 Hz, 1H), 2.75 (m, 2H), 1.96 (m, 2H), 1.81 (m, 1H), 1.69 (m, 1H), 1.36 (d, J=7 Hz, 6H).

### 17e) 6-[4-({[3-(2,6-Dichlorophenyl)-5-(1-methylethyl)-4-isoxazolyl]methyl}oxy)phenyl]-1,2,3,4-tetrahydro-1-naphthalenecarboxylic Acid

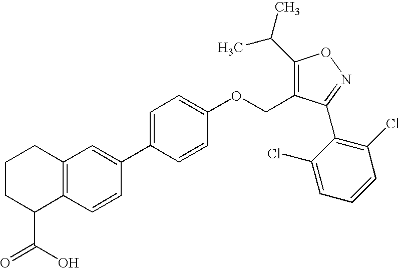

Methyl 6-[4-({[3-(2,6-dichlorophenyl)-5-(1-methylethyl)-4-isoxazolyl]methyl}oxy)phenyl]-1,2,3,4-tetrahydro-1-naphthalenecarboxylate (0.14 g, 0.254 mmol) was dissolved in a mixture of 1 N lithium hydroxide (1 mL) and 1,4-dioxane (1 mL) and stirred at room temperature. The reaction mixture was concentrated after stirring for 24 hours and the white solid was diluted with water, followed by saturated sodium hydrogensulfate. The reaction was extracted with ethyl acetate and the ethyl acetate layer washed with brine, dried over magnesium sulfate, filtered, and concentrated. The crude material was purified using hexanes:ethyl acetate (30%) to afford 0.134 g (99%) of 6-[4-({[3-(2,6-dichlorophenyl)-5-(1-methylethyl)-4-isoxazolyl]methyl}oxy)phenyl]-1,2,3,4-tetrahydro-1-naphthalenecarboxylic acid as a white foam. ¹H NMR (400 MHz, d₆-DMSO): δ 12.37 (s, 1H), 7.61 (m, 2H), 7.52 (dd, J=9, 7 Hz, 1H), 7.45 (d, J=9 Hz, 2H), 7.28 (m, 2H), 7.15 (d, J=8 Hz, 1H), 6.82 (d, J=9 Hz, 2H), 4.82 (s, 2H), 3.70 (t, J=6 Hz, 1H), 3.43 (septet, J=7 Hz, 1H), 2.74 (m, 2H), 2.00 (m, 1H), 1.85 (m, 2H), 1.69 (m, 1H), 1.31 (d, J=7 Hz, 6H). HRMS C₃₀H₂₇Cl₂NO₄ m/z 536.1395 (M+H)⁺_{Cal}; 536.1400 (M+H)⁺_{Obs}.

### Example 18

### 8-{[4-({[3-(2,6-Dichlorophenyl)-5-(1-methylethyl)-4-isoxazolyl]methyl}oxy)phenyl]amino}-2-naphthalenecarboxylic Acid

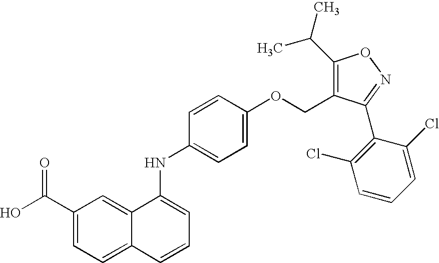

### 18a) Methyl 8-hydroxy-2-naphthalenecarboxylate

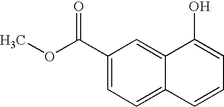

8-Hydroxy-2-naphthalenecarboxylic acid (1.96 g, 10.4 mmol) in methanol (93 mL) was stirred while thionyl chloride (1.5 mL, 21.04 mmol) was added. After addition of the thionyl chloride, the reaction mixture was heated at reflux overnight. The reaction mixture was cooled to room temperature, and concentrated to a light brown solid, then redissolved in ethyl acetate. The mixture was washed with 5% sodium bicarbonate, followed by brine, then dried over magnesium sulfate, filtered, and concentrated to afford 2.17 g (100%) of methyl 8-hydroxy-2-naphthalenecarboxylate as a light brown solid. ¹H NMR (400 MHz, d₆-DMSO): δ 10.55 (s, 1H), 8.81 (s, 1H), 7.91 (m, 2H), 7.42 (m, 2H), 6.93 (d, 7 Hz, 1H), 3.88 (s, 3H).

### 18b) Methyl 8-{[(trifluoromethyl)sulfonyl]oxy}-2-naphthalenecarboxylate

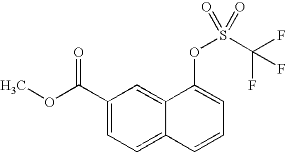

A round bottom flask containing methyl 8-hydroxy-2-naphthalenecarboxylate (2.1 g, 10.4 mmol) and pyridine (5 mL, 62.3 mmol) in dichloromethane (12 mL) was cooled to 0° C. Trifluoromethanesulfonic anhydride (2.1 mL, 12.5 mmol) in dichloromethane (5 mL) was slowly added to the reaction mixture. The reaction mixture was stirred in an ice-bath for 1.5 hours. It was then diluted with water, followed by diethyl ether. The layers were separated and the ether layer was washed with water several times, followed by brine, then dried over magnesium sulfate, filtered, and concentrated to afford a crude oil. The crude material was purified using hexanes:ethyl acetate (10% ethyl acetate) to afford 3.05 g (88%) of methyl 8-{[(trifluoromethyl)sulfonyl]oxy}-2-naphthalenecarboxylate. ¹H NMR (400 MHz, d₆-DMSO): δ 8.59 (s, 1H), 8.23 (d, J=9 Hz, 1H), 8.18 (m, 1H), 8.13 (dd, J=9, 1 Hz, 1H), 7.79 (m, 2H), 3.92 (s, 3H).

### 18c) Methyl 8-{[4-(methyloxy)phenyl]amino}-2-naphthalenecarboxylate

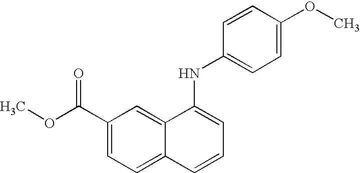

Methyl 8-{[(trifluoromethyl)sulfonyl]oxy}-2-naphthalenecarboxylate (0.5 g, 1.50 mmol), p-anisidine (0.269 g, 2.18 mmol), tris(dibenzylideneacetone)dipalladium(0) (0.06 g, 0.066 mmol), 2,2′-bis(diphenylphosphino)-1,1′-binaphthyl (0.06 g, 0.096 mmol) and cesium carbonate (0.8 g, 2.45 mmol) were heated in toluene (20 mL) at reflux for 48 hours. The reaction mixture was diluted with 1 N hydrochloric acid (50 mL) followed by ethyl acetate. The layers were separated and the organic layer was washed with brine, dried over magnesium sulfate, filtered over a pad of Celite®, and concentrated to afford a dark oil. The crude material was purified using hexanes:ethyl acetate (20% ethyl acetate) to afford 0.33 g (72%) of methyl 8-{[4-(methyloxy)phenyl]amino}-2-naphthalenecarboxylate as a red oil. ¹H NMR (400 MHz, d₆-DMSO): δ 8.98 (s, 1H), 8.35 (s, 1H), 7.92 (m, 2H), 7.38 (m, 2H), 7.13 (d, J=9 Hz, 2H), 7.03 (d, J=7 Hz, 1H), 6.90 (d, J=9 Hz, 2H), 3.89 (s, 3H), 3.72 (s, 3H).

### 18d) Methyl 8-[(4-hydroxyphenyl)amino]-2-naphthalenecarboxylate

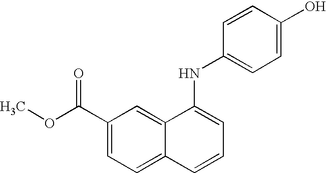

To a solution of methyl 8-{[4-(methyloxy)phenyl]amino}-2-naphthalenecarboxylate (0.3 g, 0.976 mmol) in dichloromethane (10 mL) at 0° C. was added 1 M boron tribromide (3.9 mL, 3.90 mmol) in dichloromethane dropwise. The reaction mixture was stirred at 0° C. for 4 hours and then poured into ice and stirred for several minutes. The mixture was extracted with dichloromethane and ethyl acetate. The organic layer was washed with brine, dried over magnesium sulfate, filtered, and concentrated to an orange oil. The crude oil was purified using hexanes:ethyl acetate (25% ethyl acetate) to afford 0.196 g (68%) of methyl 8-[(4-hydroxyphenyl)amino]-2-naphthalenecarboxylate. ¹H NMR (400 MHz, d₆-DMSO): δ 9.11 (s, 1H), 8.99 (s, 1H), 8.25 (s, 1H), 7.90 (m, 2H), 7.35 (t, J=8 Hz, 1H), 7.30 (d, J=8 Hz, 1H), 7.03 (d, J=9 Hz, 2H), 6.93 (d, J=8 Hz, 1H), 6.74 (d, J=9 Hz, 2H), 3.89 (s, 3H)

### 18e) 8-{[4-({[3-(2,6-Dichlorophenyl)-5-(1-methylethyl)-4-isoxazolyl]methyl}oxy)phenyl]amino}-2-naphthalenecarboxylic Acid

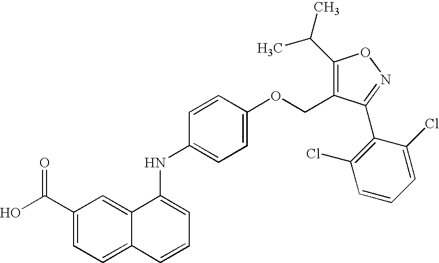

Methyl 8-[(4-hydroxyphenyl)amino]-2-naphthalenecarboxylate (0.196 g, 0.668 mmol), [3-(2,6-dichlorophenyl)-5-(1-methylethyl)-4-isoxazolyl]methanol (0.191 g, 0.668 mmol) and triphenylphosphine (0.175 g, 0.668 mmol) were added to stirred dichloromethane (10 mL) at 0° C., then diisopropyl azodicarboxylate (0.132 mL, 0.668 mmol) was slowly added to the reaction mixture. The reaction was allowed to warm to room temperature. After stirring for 2 days at room temperature, the reaction mixture was concentrated to an oil. The crude oil was partially purified by flash chromatography over silicon dioxide using hexanes:ethyl acetate (100:0 to 95:5) followed by a second flash chromatography column over silicon dioxide using hexanes:dichloromethane (50% dichloromethane) to obtain 0.1 g of impure methyl 8-{[4-({[3-(2,6-dichlorophenyl)-5-(1-methylethyl)-4-isoxazolyl]methyl}oxy)phenyl]amino}-2-naphthalenecarboxylate. The impure ester intermediate was taken on without further purification.

Methyl 8-{[4-({[3-(2,6-dichlorophenyl)-5-(1-methylethyl)-4-isoxazolyl]methyl}oxy)phenyl]amino}-2-naphthalenecarboxylate (0.1 g, 0.178 mmol) and 1 N lithium hydroxide (1 mL) were stirred in tetrahydrofuran (1 mL) for 2 days, then 1,4-dioxane (1 mL) was added and stirred reaction mixture for another day. The reaction mixture was concentrated, then diluted with water, followed by saturated sodium hydrogensulfate then the mixture was extracted with ethyl acetate. The organic layer was washed with brine, dried over magnesium sulfate, filtered, and concentrated to afford a crude material. The crude material was purified using dichloromethane:methanol (5% methanol) to afford a sample with an impurity. The partially purified sample was repurified using acetonitrile:water (50-100% acetonitrile) to afford 0.007 g (7.1%) of 8-{[4-({[3-(2,6-dichlorophenyl)-5-(1-methylethyl)-4-isoxazolyl]methyl}oxy)phenyl]amino}-2-naphthalenecarboxylic acid. ¹H NMR (400 MHz, d₆-DMSO): δ 12.95 (s, 1H), 8.92 (s, 1H), 8.30 (s, 1H), 7.92 (dd, J=8, 1 Hz, 1H), 8.87 (d, J=9 Hz, 1H), 7.62 (m, 2H), 7.54 (m, 1H), 7.38 (m, 2H), 7.03 (m, 3H), 6.73 (d, J=9 Hz, 2H), 4.75 (s, 2H), 3.41 (septet, J=7 Hz, 1H), 1.31 (d, J=7 Hz, 6H). HRMS C₃₀H₂₄Cl₂N₂O₄ m/z 547.1191 (M+H)⁺_{Cal}; 547.1195 (M+H)⁺_{Obs}.

### Example 19

### 4-{[4-({[3-(2,6-Dichlorophenyl)-5-(1-methylethyl)-4-isoxazolyl]methyl}oxy)phenyl]thio}-1-benzothiophene-2-carboxylic Acid

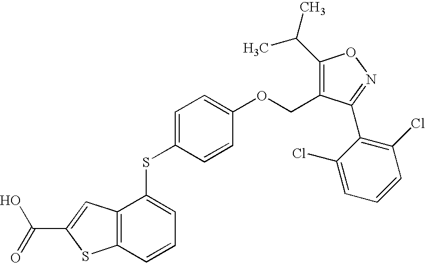

### 19a) 2-Chloro-6-{[4-(methyloxy)phenyl]thio}benzaldehyde

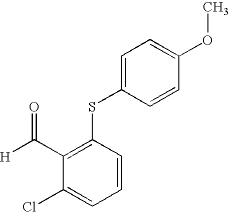

To a solution of 2-chloro-6-nitrobenzaldehyde (2 g, 10.8 mmol) and 4-methoxythiophenol (1.3 mL, 10.8 mmol) in N,N-dimethylformamide (22 mL) was added potassium carbonate (1.49 g, 10.8 mmol). The reaction mixture was stirred at room temperature. After 2 days of stirring, the reaction mixture was poured into ice-water, then filtered, and ethyl acetate was added to the filtrate. The layers were separated. The organic layer was washed with brine, dried over magnesium sulfate, filtered, and concentrated to afford a crude material. The crude material was purified using hexanes:ethyl acetate (15% ethyl acetate) to afford 1.2 g (40%) of 2-chloro-6-{[4-(methyloxy)phenyl]thio}benzaldehyde as a yellow solid. ¹H NMR (400 MHz, d₆-DMSO): δ 10.51 (s, 1H) 7.46 (d, J=9 Hz, 2H), 7.40 (t, J=8 Hz, 1H), 7.33 (d, J=8 Hz, 1H), 7.08 (d, J=9 Hz, 2H), 6.63 (d, J=8 Hz, 1H), 3.80 (s, 3H).

### 19b) Methyl 4-{[4-(methyloxy)phenyl]thio}-1-benzothiophene-2-carboxylate

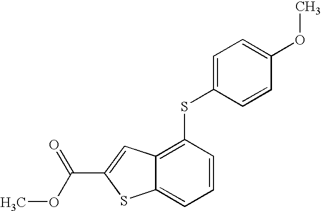

To a solution of methyl thioglycolate (0.385 mL, 4.30 mmol) and 2-chloro-6-{[4-(methyloxy)phenyl]thio}benzaldehyde (1.2 g, 4.30 mmol) in N,N-dimethylformamide (12 mL) was added sodium methoxide (0.233 g, 4.30 mmol). The reaction mixture was stirred at room temperature. Excess sodium methoxide (0.23 g, 4.30 mmol) and methyl thioglycolate (0.39 mL, 4.30 mmol) were added to the reaction mixture after 3 days of stirring. Stirring continued. There was no change in the reaction mixture. The reaction mixture was heated at 65° C. for 3 days. The reaction mixture was poured into ice-water and extracted with ethyl acetate. The organic layer was washed with brine, dried over magnesium sulfate, filtered, and concentrated to an oil. The crude oil was purified using hexanes:acetone (20% acetone) to afford 0.399 g (28%) of methyl 4-{[4-(methyloxy)phenyl]thio}-1-benzothiophene-2-carboxylate. ¹H NMR (400 MHz, d₆-DMSO): δ 8.09 (s, 1H) 7.95 (d, J=8 Hz, 1H), 7.44 (t, J=8 Hz, 1H), 7.40 (d, J=9 Hz, 2H), 7.07 (d, J=8 Hz, 1H), 7.00 (d, J=9 Hz, 2H), 3.87 (s, 3H), 3.75 (s, 3H).

### 19c) Methyl 4-[(4-hydroxyphenyl)thio]-1-benzothiophene-2-carboxylate

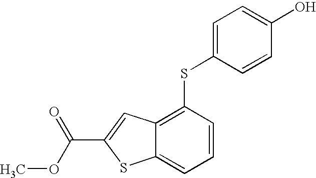

To a solution of methyl 4-{[4-(methyloxy)phenyl]thio}-1-benzothiophene-2-carboxylate (0.39 g, 1.18 mmol) in dichloromethane (12 mL) at 0° C. was added 1M boron tribromide (4.72 mL, 4.72 mmol) in dichloromethane drop wise. The reaction mixture was stirred at 0° C. for 5.5 hours, and then poured into ice and stirred for several minutes. The mixture was extracted with ethyl acetate and the organic layer was washed with brine, dried over magnesium sulfate, filtered, and concentrated to an orange oil. The crude oil was purified using hexanes:ethyl acetate (50% ethyl acetate) to afford 0.3 g (81%) of methyl 4-[(4-hydroxyphenyl)thio]-1-benzothiophene-2-carboxylate. ¹H NMR (400 MHz, d₆-DMSO): δ 9.89 (s, 1H), 8.09 (s, 1H) 7.91 (d, J=8 Hz, 1H), 7.41 (t, J=8 Hz, 1H), 7.32 (d, J=9 Hz, 2H), 6.98 (d, J=7 Hz, 1H), 6.82 (d, J=9 Hz, 2H), 3.87 (s, 3H).

### 19d) Methyl 4-{[4-({[3-(2,6-dichlorophenyl)-5-(1-methylethyl)-4-isoxazolyl]methyl}oxy)phenyl]thio}-1-benzothiophene-2-carboxylate

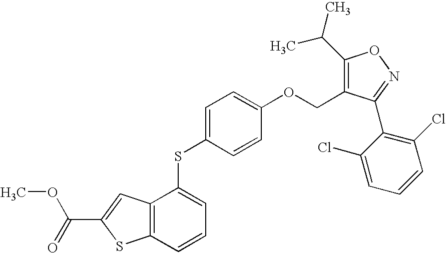

Methyl 4-[(4-hydroxyphenyl)thio]-1-benzothiophene-2-carboxylate (0.3 g, 0.948 mmol), [3-(2,6-dichlorophenyl)-5-(1-methylethyl)-4-isoxazolyl]methanol (0.27 g, 0.948 mmol) and triphenylphosphine (0.25 g, 0.948 mmol) were stirred in dichloromethane (15 mL) at 0° C. then diisopropyl azodicarboxylate (0.187 mL, 0.948 mmol) was slowly added to the reaction mixture. The reaction was allowed to warm to room temperature. After stirring for 2 days at room temperature, the reaction mixture was concentrated to an oil. The crude oil was purified using hexanes:ethyl acetate (40% ethyl acetate) to afford 0.45 g (82%) of methyl 4-{[4-({[3-(2,6-dichlorophenyl)-5-(1-methylethyl)-4-isoxazolyl]methyl}oxy)phenyl]thio}-1-benzothiophene-2-carboxylate. ¹H NMR (400 MHz, d₆-DMSO): δ 8.07 (s, 1H), 7.96 (d, J=8 Hz, 1H), 7.58 (m, 2H), 7.50 (dd, J=9, 7 Hz, 1H), 7.43 (t, J=8 Hz, 1H), 7.30 (d, J=9 Hz, 2H), 7.07 (d, J=8 Hz, 1H), 6.82 (d, J=9 Hz, 2H), 4.82 (s, 2H), 3.86 (s, 3H), 3.41 (septet, J=7 Hz, 1H), 1.29 (d, J=7 Hz, 6H).

### 19e) 4-{[4-({[3-(2,6-Dichlorophenyl)-5-(1-methylethyl)-4-isoxazolyl]methyl}oxy)phenyl]thio}-1-benzothiophene-2-carboxylic Acid

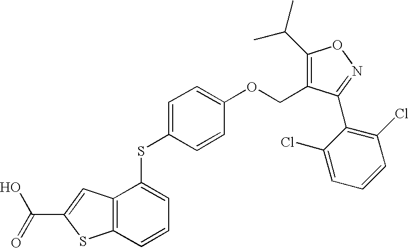

Methyl 4-{[4-({[3-(2,6-dichlorophenyl)-5-(1-methylethyl)-4-isoxazolyl]methyl}oxy)phenyl]thio}-1-benzothiophene-2-carboxylate (0.418 g, 0.715 mmol) and 1 N lithium hydroxide (2.5 mL) were added to a stirred mixture of tetrahydrofuran (1.5 mL) and 1,4-dioxane (1 mL). The mixture was stirred for 6.5 hours. The reaction mixture was concentrated, then diluted with water, followed by saturated sodium hydrogensulfate, then the mixture was extracted with ethyl acetate. The organic layer was washed with brine, dried over magnesium sulfate, filtered, and concentrated to afford crude material. The crude material was purified using hexanes:ethyl acetate (50% ethyl acetate) to afford 0.18 g (44%) of 4-{[4-({[3-(2,6-dichlorophenyl)-5-(1-methylethyl)-4-isoxazolyl]methyl}oxy)phenyl]thio}-1-benzothiophene-2-carboxylic acid. ¹H NMR (400 MHz, d₆-DMSO): δ 13.62 (s, 1H), 7.99 (s, 1H), 7.94 (d, J=8 Hz, 1H), 7.58 (m, 2H), 7.50 (dd, J=9, 7 Hz, 1H), 7.41 (t, J=8 Hz, 1H), 7.29 (d, J=9 Hz, 2H), 7.07 (d, J=8 Hz, 1H), 6.82 (d, J=9 Hz, 2H), 4.82 (s, 2H), 3.41 (septet, J=7 Hz, 1H), 1.29 (d, J=7 Hz, 6H). HRMS C₂₈H₂₁C₁₂NO₄S₂ m/z 570.0367 (M+H)⁺_{Cal}; 570.0373 (M+H)⁺_{Obs}.

### Example 20

### Ethyl 6-[4-({[3-(2,6-dichlorophenyl)-5-(1-methylethyl)-4-isoxazolyl]methyl}oxy)phenyl]-1,2-benzisoxazole-3-carboxylate

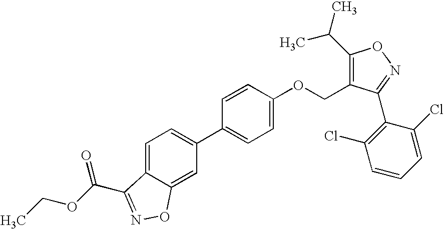

### 20a) Ethyl 6-(4-hydroxyphenyl)-1,2-benzisoxazole-3-carboxylate

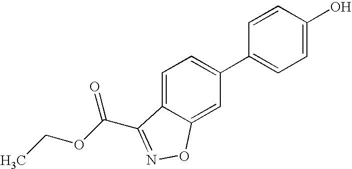

Ethyl 6-bromo-1,2-benzisoxazole-3-carboxylate (0.34 g, 1.26 mmol), (4-hydroxyphenyl)boronic acid (0.21, 1.51 mmol), tetrakis(triphenylphosphine)palladium (0) (0.06 g, 0.05 mmol) and 2 M sodium carbonate (5 mL) in 1,2-dimethoxyethane (6 mL) were stirred at 80° C. for 1 hour. The reaction mixture was diluted with water, followed by ethyl acetate. The layers were separated and the ethyl acetate layer was washed with brine, dried over magnesium sulfate, filtered, and concentrated to afford the crude material. The crude material was purified using hexanes:ethyl acetate (0 to 40% ethyl acetate) to afford a mixture of desired product, plus the free acid of the desired product, 6-(4-hydroxyphenyl)-1,2-benzisoxazole-3-carboxylic acid. All the fractions were combined and concentrated. The mixture was then dissolved in ethanol. Thionyl chloride was added, and the reaction mixture was reflux for 8 days. The reaction mixture was cooled to room temperature and concentrated. The concentrated material was diluted with 5% sodium bicarbonate, and extracted with ethyl acetate. The ethyl acetate layer was washed with water followed by brine, dried over magnesium sulfate, filtered, and concentrated. The crude material was purified using hexanes:ethyl acetate (0 to 100% ethyl acetate) to afford 0.044 g (13%) of ethyl 6-(4-hydroxyphenyl)-1,2-benzisoxazole-3-carboxylate. ¹H NMR (400 MHz, d₆-DMSO): δ 9.76 (s, 1H), 8.05 (m, 2H), 7.79 (dd, J=8, 1 Hz, 1H), 7.65 (d, J=9 Hz, 2H), 6.88 (d, J=9 Hz, 2H), 4.47 (q, J=7 Hz, 2H), 1.39 (t, J=7 Hz, 3H).

### 20b) Ethyl 6-[4-({[3-(2,6-dichlorophenyl)-5-(1-methylethyl)-4-isoxazolyl]methyl}oxy)phenyl]-1,2-benzisoxazole-3-carboxylate

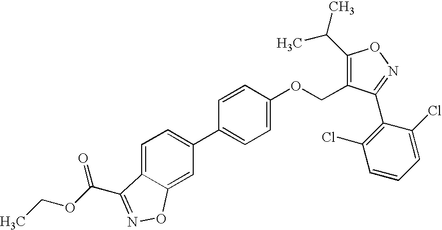

Ethyl 6-(4-hydroxyphenyl)-1,2-benzisoxazole-3-carboxylate (0.044 g, 0.155 mmol), [3-(2,6-dichlorophenyl)-5-(1-methylethyl)-4-isoxazolyl]methanol (0.044 g, 0.155 mmol) and triphenylphosphine (0.041 g, 0.155 mmol) were added to stirred dichloromethane (4 mL) at 0° C., then diisopropyl azodicarboxylate (0.031 mL, 0.155 mmol) was added slowly to the reaction mixture. The reaction was allowed to warm to room temperature. After stirring for 3 days at room temperature, the reaction mixture was concentrated to a yellowish oil. The crude oil was purified using hexanes:ethyl acetate (20% ethyl acetate) to afford 0.056 g (65%) of ethyl 6-[4-({[3-(2,6-dichlorophenyl)-5-(1-methylethyl)-4-isoxazolyl]methyl}oxy)phenyl]-1,2-benzisoxazole-3-carboxylate. ¹H NMR (400 MHz, d₆-DMSO): δ 8.10 (s, 1H), 8.05 (d, J=9 Hz, 1H), 7.79 (dd, J=9, 1 Hz, 1H), 7.68 (d, J=9 Hz, 2H), 7.61 (m, 2H), 7.53 (dd, J=9, 7 Hz, 1H), 6.91 (d, J=9 Hz, 2H), 4.87 (s, 2H), 4.47 (q, J=7 Hz, 2H), 3.45 (septet, J=7 Hz, 1H), 1.38 (t, J=7 Hz, 3H), 1.32 (d, J=7 Hz, 6H).

### Example 21

### 2-[4-({[3-(2,6-Dichlorophenyl)-5-(1-methylethyl)-4-isoxazolyl]methyl}oxy)phenyl]-1H-benzimidazole-4-carboxylic Acid

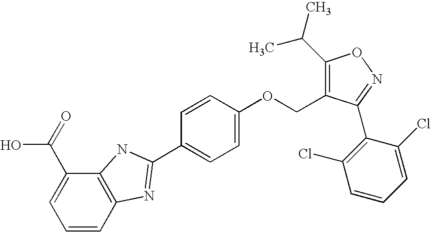

### 21a) Methyl 2,3-diaminobenzoate

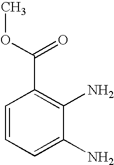

10% Palladium on carbon (0.25 g) was placed in a 3-neck round bottom flask then flushed with nitrogen and evacuated. This was repeated several times, then ethanol (100 mL) was added to the flask. Partially dissolved methyl 2-amino-3-nitrobenzoate (3.5 g, 17.8 mmol) in ethanol (60 mL) was added, and the reaction mixture purged with nitrogen and evacuated. The reaction mixture was left to stir under hydrogen (1.6 L). After stirring for 24 h, the reaction mixture was filtered over a pad of Celite®, washed with ethanol and the filtrate was concentrated to afford 3.4 g (100%) of methyl 2,3-diaminobenzoate. ¹H NMR (400 MHz, d₆-DMSO): δ 7.07 (dd, J=8, 1 Hz, 1H), 6.67 (m, 1H), 6.36 (t, J=8 Hz, 1H), 6.17 (br s, 2H), 4.74 (br s, 2H), 3.73 (s, 3H).

### 21b) Methyl 2-[4-(methyloxy)phenyl]-1H-benzimidazole-4-carboxylate

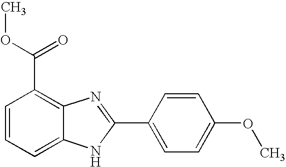

To a solution of triphenylphosphine oxide (12.6 g, 45.1 mmol) in 1,2-dichloroethane (68 mL) at 0° C. was added trifluoromethanesulfonic anhydride (3.8 mL, 22.6 mmol). The reaction mixture was stirred at 0° C. for 20 minutes then methyl 2,3-diaminobenzoate (1.5 g, 9.03 mmol) and 4-(methyloxy)benzoic acid (1.72 g, 11.28 mmol) in dichloromethane (23 mL) were slowly added to the reaction mixture at 0° C. The reaction mixture was allowed to stir at 0° C. for 2 hours. The reaction mixture was diluted with 5% sodium bicarbonate, followed by water and extracted with dichloromethane. The organic layer was washed with brine, dried over magnesium sulfate, filtered, and concentrated. The crude material was purified using hexanes:ethyl acetate (50% ethyl acetate) to afford partially purified material. This partially purified material was repurified using hexanes:ethyl acetate (50% ethyl acetate) to afford a mixture of uncyclized and cyclized compounds (0.95 g). The mixture of uncyclized and cyclized compounds was dissolved in acetic acid and heated at 120° C. for 40 minutes. The mixture was cooled to room temperature, poured into ice-cold water and extracted with dichloromethane. The organic layer was washed with saturated sodium bicarbonate, followed by brine, then it was dried over sodium sulfate, filtered, and concentrated. The crude material was purified using hexanes:ethyl acetate (70% ethyl acetate) to afford 0.64 g (26%) of methyl 2-[4-(methyloxy)phenyl]-1H-benzimidazole-4-carboxylate. ¹H NMR (400 MHz, C₆D₆): δ 10.35 (s, 1H), 8.01 (d, J=8 Hz, 1H), 7.84 (d, J=8 Hz, 1H), 7.79 (d, J=9 Hz, 2H), 7.00 (t, J=8 Hz, 1H), 6.62 (d, J=9 Hz, 2H), 3.46 (s, 3H), 3.17 (s, 3H).

### 21c) Methyl 2-(4-hydroxyphenyl)-1H-benzimidazole-4-carboxylate

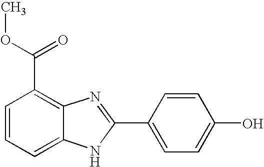

To a solution of methyl 2-[4-(methyloxy)phenyl]-1H-benzimidazole-4-carboxylate (0.30 g, 1.06 mmol) in dichloromethane (7 mL) at 0° C. was added 1 M boron tribromide (5.3 mL, 5.31 mmol) in dichloromethane, slowly. The reaction mixture was stirred at the above temperature for approximately 2 hours, and then poured into ice and stirred for several minutes. The mixture was extracted with ethyl acetate and the organic layer was washed with brine, dried over magnesium sulfate, filtered, and concentrated to give a brown solid. The crude material was purified using hexanes:ethyl acetate (50% ethyl acetate) to afford 0.140 g (49%) of methyl 2-(4-hydroxyphenyl)-1H-benzimidazole-4-carboxylate. ¹H NMR (400 MHz, d₆-DMSO): δ 10.32 (br s, 1H), 8.10 (d, J=9 Hz, 2H), 7.94 (d, J=8 Hz, 1H), 7.89 (d, J=8 Hz, 1H), 7.44 (t, J=8 Hz, 1H), 6.97 (d, J=9 Hz, 2H), 3.97 (s, 3H).

### 21d) Methyl 2-[4-({[3-(2,6-dichlorophenyl)-5-(1-methylethyl)-4-isoxazolyl]methyl}oxy)phenyl]-1H-benzimidazole-4-carboxylate

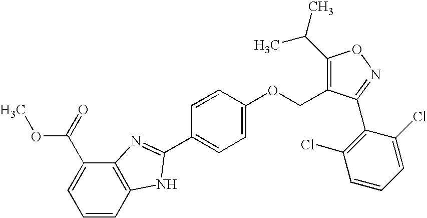

Methyl 2-(4-hydroxyphenyl)-1H-benzimidazole-4-carboxylate (0.14 g, 0.52 mmol), [3-(2,6-dichlorophenyl)-5-(1-methylethyl)-4-isoxazolyl]methanol (0.15 g, 0.52 mmol) and triphenylphosphine (0.14 g, 0.52 mmol) were added to stirred dichloromethane (6 mL) at 0° C., then diisopropyl azodicarboxylate (0.103 mL, 0.52 mmol) in dichloromethane (2 mL) was slowly added to the reaction mixture. The reaction mixture was allowed to warm to room temperature. After stirring for 18 hours at room temperature, the reaction mixture was concentrated. The crude oil was purified using hexanes:ethyl acetate (50% ethyl acetate) to afford partially pure methyl 2-[4-({[3-(2,6-dichlorophenyl)-5-(1-methylethyl)-4-isoxazolyl]methyl}oxy)phenyl]-1H-benzimidazole-4-carboxylate (0.14 g). The impure ester was used without further purification. ES-LCMS (m/z) 534 (M−H⁺)⁻.

### 21e) 2-[4-({[3-(2,6-Dichlorophenyl)-5-(1-methylethyl)-4-isoxazolyl]methyl}oxy)phenyl]-1H-benzimidazole-4-carboxylic Acid

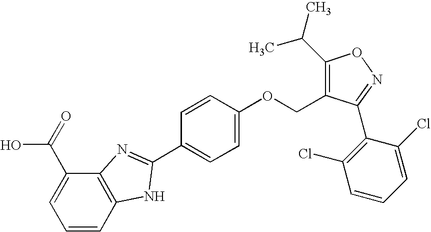

Methyl 2-[4-({[3-(2,6-dichlorophenyl)-5-(1-methylethyl)-4-isoxazolyl]methyl}oxy)phenyl]-1H-benzimidazole-4-carboxylate (0.14 g, 0.260 mmol) and 1 N lithium hydroxide (1 mL) were stirred in tetrahydrofuran (1 mL) for 24 hours, then 1,4-dioxane (1 mL) was added. The reaction mixture was stirred for another 24 hours. The reaction mixture was concentrated, then diluted with saturated sodium hydrogensulfate, followed by water. The mixture was then extracted with ethyl acetate. The organic layer was washed with brine, dried over magnesium sulfate, filtered, and concentrated to afford a crude material. The crude material was purified using dichloromethane:methanol (9% methanol) to afford a partially pure sample. The impure sample was repurified using acetonitrile:water (50-100% acetonitrile) to afford 0.0096 g (7%) of 2-[4-({[3-(2,6-dichlorophenyl)-5-(1-methylethyl)-4-isoxazolyl]methyl}oxy)phenyl]-1H-benzimidazole-4-carboxylic acid as a white powder. ¹H NMR (400 MHz, d₆-DMSO): δ 8.15 (d, J=9 Hz, 2H), 7.87 (d, J=8 Hz, 1H), 7.81 (d, J=8 Hz, 1H), 7.62 (m, 2H), 7.53 (dd, J=9, 7 Hz, 1H), 7.34 (br t, J=8 Hz, 1H), 6.95 (d, J=9 Hz, 2H), 4.92 (s, 2H), 3.47 (septet, J=7 Hz, 1H), 1.33 (d, J=7 Hz, 6H). HRMS C₂₇H₂₁Cl₂N₃O₄ m/z 522.0987 (M+H)⁺_{Cal}; 522.0984 (M+H)⁺_{Obs}.

### Biological Example 22

### FXR Cofactor Binding Assay

Determination of a ligand mediated cofactor peptide interaction to quantify ligand binding to the nuclear receptor Farnesoid X Receptor (FXR). The method measures the ability of putative ligands to modulate the interaction between the purified bacterial expressed FXRα ligand binding domain (LBD) and a synthetic biotinylated peptide based on residues 676-700 of steroid receptor coactivator-1 (SRC-1) (LXXLL-containing domain-2 where L is the amino acid leucine and X indicates any other amino acid (LCD2), 676-700). The sequence of the SRC-1 peptide used is as published in Iannone, M. A., et al., 2001 Cytometry 44:326-337 where the N-terminus was biotinylated (B) and the C-terminus was amidated. Detection of the associated complex was measured by time resolved fluorescence (TRF). The purified LBD of FXR was labeled with biotin then mixed with stoichiometric amounts of allophycocyanin (APC) labeled streptavidin (Molecular Probes). The biotinylated peptide was then mixed with a ½ stoichiometric amount of europium labeled streptavidin (Wallac Inc). Each was then blocked with a 5 fold excess of biotin and allowed to equilibrate for 15 min. Equimolar amounts of receptor and peptide were mixed together and were allowed to equilibrate for at least 30 min prior to the addition to either a variable or constant concentrations of the sample for which the affinity is to be determined. After equilibration, the time-resolved fluorescent signal was quantitated using a fluorescent plate reader. The affinity of the test compound was estimated from a plot of fluorescence versus concentration of test compound added.

A basal level of FXR: peptide formation is observed in the absence of added ligand. Ligands that promote the complex formation induce a concentration-dependent increase in time-resolved fluorescent signal. Compounds which bind equally well to both monomeric FXR and to the FXR: peptide complex would be expected to give no change in signal, whereas ligands which bind preferentially to the monomeric receptor would be expected to induce a concentration-dependent decrease in the observed signal.

### Methods & Materials

Advance Preparation: Human Farnesoid X Receptor α Ligand Binding Domain Human FXRα Ligand Binding Domain (FXRα LBD) was expressed in E. coli strain BL21 (DE3) as an amino-terminal polyhistidine tagged fusion protein. Expression was under the control of an isopropyl-β-D-thiogalactopyranoside (IPTG) inducible T7 promoter. DNA encoding this recombinant protein is subcloned into the pRSET-A expression vector (Invitrogen). The coding sequence of Human FXRα LBD was derived from Genbank accession number U 68233 (amino acids 237 to 472).

Ten-liter fermentation batches were grown in Rich PO₄ media with 0.1 mg/mL Ampicillin at 25° C. for 12 hours, cooled to 9° C. and held at that temperature for 36 hours to a density of OD₆₀₀=14. At this cell density, 0.25 mM IPTG is added and induction proceeded for 24 hours at 9° C., to a final OD₆₀₀=16. Cells are harvested by centrifugation (20 minutes, 3500× gravity, 4° C.), and concentrated cell slurries were stored in phosphate buffered saline (PBS) at −8° C.

### Purification of Receptor Ligand Binding Domain

Routinely, 30-40 g cell paste (equivalent to 2-3 liters of the fermentation batch) was resuspended in 200-250 mL Tris buffered saline (TBS), pH 7.2 (25 mM Tris-hydroxymethylamino methane (Tris), 150 mM sodium chloride). Cells were lysed by passing 3 times through a French Press and cell debris was removed by centrifugation (30 minutes, 20,000× gravity, 4° C.). The cleared supernatant was filtered through course pre-filters, and TBS, pH 7.2, 500 mM imidazole was added to obtain a final imidazole concentration of 50 mM. This lysate was loaded onto a column (6×8 cm) packed with Sepharose [Ni⁺⁺ charged] Chelation resin (Pharmacia) and pre-equilibrated with TBS pH 7.2/50 mM imidazole. After washing to baseline absorbance with equilibration buffer, the column was washed with one column volume of TBS pH 7.2 containing 90 mM imidazole. FXRα LBD was eluted directly with 365 mM imidazole. Column fractions were pooled and dialyzed against TBS, pH 7.2, containing 0.5 mM EDTA and 5 mM DTT. The dialyzed protein sample was concentrated using Centri-prep 10 K (Amicon) and subjected to size exclusion, using a column (3×90 cm) packed with Sepharose S-75 resin (Pharmacia) pre-equilibrated with TBS, pH 7.2, containing 0.5 mM ethylene diamine tetraacetic acid (EDTA) and 5 mM dithiothreitol (DTT).

### Biotinylation of FXR

Purified FXRα LBD was desalted/buffer exchanged using PD-10 gel filtration columns into PBS [100 mM Na₂PO₄, pH 7.2, 150 mM NaCl]. FXRα LBD was diluted to approximately 60 μM in PBS and five-fold molar excess of NHS-LC-Biotin (Pierce) is added in a minimal volume of PBS. This solution was incubated with gentle mixing for 30 minutes at room temperature. The biotinylation modification reaction was stopped by the addition of 2000× molar excess of Tris-HCl, pH 8. The modified FXRα LBD was dialyzed against 4 buffer changes, each of at least 50 volumes, PBS containing 5 mM DTT, 2 mM EDTA and 2% sucrose. The biotinylated FXRα LBD was then subjected to mass spectrometric analysis to reveal the extent of modification by the biotinylation reagent. In general, approximately 95% of the protein had at least a single site of biotinylation; and the overall extent of biotinylation followed a normal distribution of multiple sites, ranging from zero to four.

### Preparation of Streptavidin-(Europium Chelate)-SRC 1: Streptavdin-(APC)-FXR Complex

Biotinylated SRC-1 (LCD2, 676-700) peptide and a ½ stoichiometric amount of streptavidin-conjugated europium chelate was incubated in assay buffer containing 10 mM DTT for at least 30 minutes. A second solution of stoichiometric amounts of biotinylated FXR and streptavidin-conjugated APC was incubated in assay buffer containing 10 mM DTT for at least 30 minutes. Each solution was then blocked with a 5 fold molar excess of biotin and allowed to equilibrate for at least 30 min. The labeled receptor and cofactor were mixed and again allowed to equilibrate for at least 30 min, added to the compound plate, utilizing e.g., a Titertek Multidrop 384.

Materials:

Assay Buffer: 50 mM 3-(N-morpholino)propanesulfonic acid (MOPS) pH 7.5, 50 mM NaF, 50 μM 3-[(3-cholamidopropyl)-demethylammonio]-1-propanesulfonate (CHAPS), 0.1 mg/ml Fraction 5 fatty acid free bovine serum albumin (BSA), 1 mM ethylenediaminetetraacetic acid (EDTA). Solid DTT is added to the assay buffer to a final concentration of 10 mM just before use in the assay. BSA, fatty acid free

DTT

NaF

Europium labeled Streptavidin: (Wallac CR28-100)

384 Well Plates

### Methods:

### Experimental Details:

Test compounds and controls were serial diluted in DMSO and 0.1 μL at the desired concentration were added to a 384 well plate.

To each well to be assayed a previously prepared solution of FXR-APC and Europium labeled SRC1 was added to 0.1 μL of test compound and controls for a final assay volume of 10 μL.

The plates were incubated for at least 1 hour at room temperature and the fluorescent signal determined in a Fluorescence Reader in a time resolved mode utilizing e.g., a Wallac Viewlux Imager or Wallac Victor Multilabel counter.

Data Reduction:

For each concentration of test compound, the results of each test well was expressed as % of control, C, calculated according to eq. 1.

$$C = 100 * \frac{{F}_{sample} - {F}_{basal}}{{F}_{std} - {F}_{basal}} \qquad ( 1 )$$

where Fₛₐₘₚₗₑ is the signal observed in a particular sample well, F_{std} is the signal observed in the presence of control agonist and F_{basal} is the count rate observed in the presence of no ligand. The values used for F_{std} and F_{basal} are averages of the corresponding control wells included on every plate. The results are reported in Table 1 below. In Table 1, + indicates a pEC₅₀ of 5-5.99; ++ indicates a pEC₅₀ 6-6.99 and +++ indicates a pEC₅₀ greater than 7.

TABLE 1

| Example | Activity (pEC₅₀) |
|---|---|
| 1 | +++ |
| 2 | ++ |
| 3 | +++ |
| 4 | +++ |
| 5 | ++ |
| 6 | ++ |
| 7 | ++ |
| 8 | + |
| 9 | + |
| 10 | ++ |
| 11 | +++ |
| 12 | ++ |
| 13 | ++ |
| 14 | +++ |
| 15 | ++ |
| 16 | ++ |
| 17 | ++ |
| 18 | ++ |
| 19 | ++ |
| 20 | <4.70 |
| 21 | + |

1. A compound of formula (I): 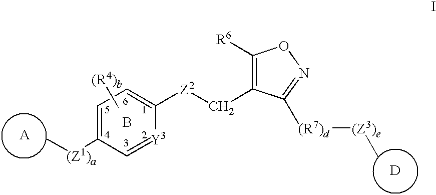
wherein:
Ring A is selected from 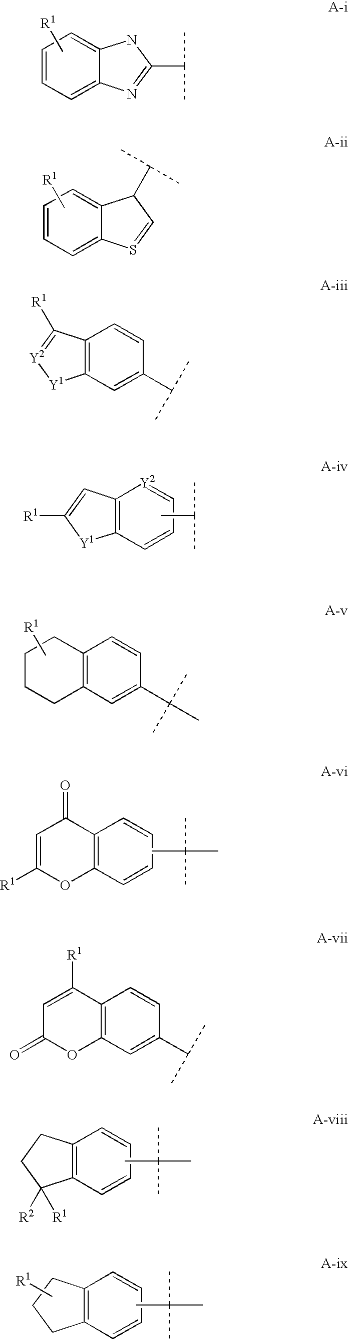
wherein
R¹ is selected from —CO₂H, —C(O)NH₂, —CO₂alkyl, and an acid equivalent group;
R² is H or —OH;
Y¹ is selected from —CH₂—, —NH—, —O— and —S—;
Y² is selected from —CH— and —NH—; or
Ring A is a substituted naphthalene when a=1; 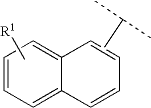
Z¹ is —NH— or —S—;
a is 0 or 1;
each R⁴ is selected from halo, alkyl and fluoroalkyl;
b is 0, 1 or 2, except that when b is 2 and Y³ is C, R⁴ is not bound at position 2 or
6 of Ring B;
Y³ is —N— or —CH—;
Z² is —O—, —S— or —N(R⁵)—, wherein R⁵ is H or alkyl;
R⁶ is selected from alkyl, 2,2,2-trifluoroethyl, C₃₋₆cycloalkyl, alkenyl, C₃₋₆cycloalkenyl and fluoro-substituted C₃₋₆cycloalkyl;
R⁷ is —C₁₋₃alkylene-,
Z³ is —O—, —S(O)_{S}, or —NH—, where c is 0, 1 or 2;
d and e are both 0 or d is 1 and e is 0 or 1;
Ring D is selected from C₃₋₆cycloalkyl and a moiety selected from formula D-i, D-ii and D-iii: 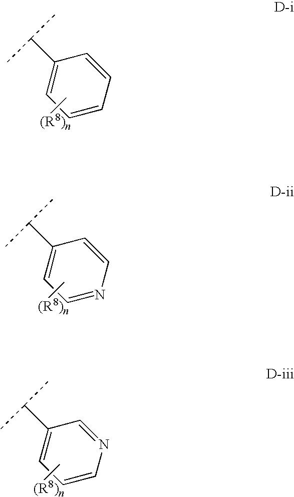
wherein
n is 0, 1, 2 or 3;
each R⁸ is the same or different and is independently selected from halo, alkyl, alkenyl, —O-alkyl, haloalkyl, —O-haloalkyl, hydroxyl substituted alkyl, and —OCF₃;
and pharmaceutically acceptable salts thereof.

2. The compound according to claim 1 wherein Ring A is A-iii:

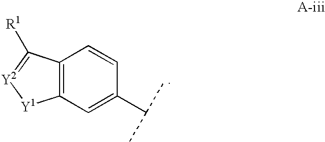

3. The compound according to claim 1 where Ring A is

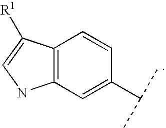

4. The compound according to claim 1 wherein Ring A is A-iv:

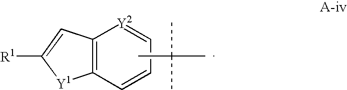

5. The compound according to claim 1 wherein Ring A is

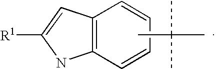

6. The compound according to claim 1 wherein R¹ is —CO₂H.

7. The compound according to claim 1 wherein a is 0.

8. The compound according to claim 1 wherein b is 0.

9. The compound according to claim 1 wherein Y³ is —CH—.

10. The compound according to claim 1 wherein Z² is —O—.

11. The compound according to claim 1 wherein R⁶ is alkyl, 2,2,2-trifluoroethyl or C₃₋₆cycloalkyl.

12. The compound according to claim 1 wherein R⁶ is isopropyl.

13. The compound according to claim 1 wherein d and e are both 0.

14. The compound according to claim 1 wherein d is 1 and R⁷ is methylene or ethylene.

15. The compound according to claim 1 wherein Ring D is a moiety of formula D-i

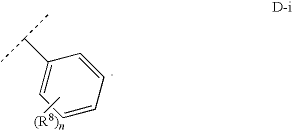

16. The compound according to claim 1 wherein Ring D is a moiety of formula D-i, n is 1, 2 or 3 and each R⁸ is halo or alkyl.

17. The compound according to claim 1 wherein n is 2.

18. The compound according to claim 1 wherein Ring D is a moiety of formula D-i, n is 2 and each R⁸ is halo or alkyl.

19. The compound according to claim 1 wherein n is 1, 2 or 3 and R⁸ is halo or alkyl.

20. A compound selected from
5-[4-({[3-(2,6-Dichlorophenyl)-5-(1-methylethyl)-4-isoxazolyl]methyl}oxy)phenyl]-1H-indole-2-carboxylic acid;
6-[4-({[3-(2,6-Dichlorophenyl)-5-(1-methylethyl)-4-isoxazolyl]methyl}oxy)phenyl]-2,3-dihydro-1H-indene-1-carboxylic acid;
6-[4-({[3-(2,6-Dichlorophenyl)-5-(1-methylethyl)-4-isoxazolyl]methyl}oxy)phenyl]-1H-indole-3-carboxylic acid;
5-[4-({[3-(2,6-Dichlorophenyl)-5-(1-methylethyl)-4-isoxazolyl]methyl}oxy)phenyl]-1-benzofuran-2-carboxylic acid;
6-[4-({[3-(2,6-Dichlorophenyl)-5-(1-methylethyl)-4-isoxazolyl]methyl}oxy)phenyl]-1H-indole-2-carboxylic acid;
5-[4-({[3-(2,6-Dichlorophenyl)-5-(1-methylethyl)-4-isoxazolyl]methyl}oxy)phenyl]-2,3-dihydro-1H-indene-2-carboxylic acid;
5-[4-({[3-(2,6-Dichlorophenyl)-5-(1-methylethyl)-4-isoxazolyl]methyl}oxy)phenyl]-1-hydroxy-2,3-dihydro-1H-indene-1-carboxylic acid;
6-[4-({[3-(2,6-Dichlorophenyl)-5-(1-methylethyl)-4-isoxazolyl]methyl}oxy)phenyl]-1H-indazole-3-carboxylic acid;
3-[4-({[3-(2,6-Dichlorophenyl)-5-(1-methylethyl)-4-isoxazolyl]methyl}oxy)phenyl]-1-benzothiophene-5-carboxylic acid;
5-[4-({[3-(2,6-Dichlorophenyl)-5-(1-methylethyl)-4-isoxazolyl]methyl}oxy)phenyl]thieno[3,2-b]pyridine-2-carboxylic acid;
6-[4-({[3-(2,6-Dichlorophenyl)-5-(1-methylethyl)-4-isoxazolyl]methyl}oxy)phenyl]-1-benzothiophene-3-carboxylic acid;
5-[4-({[3-(2,6-Dichlorophenyl)-5-(1-methylethyl)-4-isoxazolyl]methyl}oxy)phenyl]-1-benzothiophene-2-carboxylic acid;
6-[6-({[3-(2,6-Dichlorophenyl)-5-(1-methylethyl)-4-isoxazolyl]methyl}oxy)-3-pyridinyl]-1H-indole-3-carboxylic acid;
6-[4-({[3-(2,6-Dichlorophenyl)-5-(1-methylethyl)-4-isoxazolyl]methyl}oxy)phenyl]-4-oxo-4H-chromene-2-carboxylic acid;
7-[4-({[3-(2,6-Dichlorophenyl)-5-(1-methylethyl)-4-isoxazolyl]methyl}oxy)phenyl]-2-oxo-2H-chromene-4-carboxylic acid;
7-[4-({[3-(2,6-Dichlorophenyl)-5-(1-methylethyl)-4-isoxazolyl]methyl}oxy)phenyl]-4-oxo-4H-chromene-2-carboxylic acid;
6-[4-({[3-(2,6-Dichlorophenyl)-5-(1-methylethyl)-4-isoxazolyl]methyl}oxy)phenyl]-1,2,3,4-tetrahydro-1-naphthalenecarboxylic acid;
8-{[4-({[3-(2,6-Dichlorophenyl)-5-(1-methylethyl)-4-isoxazolyl]methyl}oxy)phenyl]amino}-2-naphthalenecarboxylic acid;
4-{[4-({[3-(2,6-Dichlorophenyl)-5-(1-methylethyl)-4-isoxazolyl]methyl}oxy)phenyl]thio}-1-benzothiophene-2-carboxylic acid;
Ethyl 6-[4-({[3-(2,6-dichlorophenyl)-5-(1-methylethyl)-4-isoxazolyl]methyl}oxy)phenyl]-1,2-benzisoxazole-3-carboxylate;
2-[4-({[3-(2,6-Dichlorophenyl)-5-(1-methylethyl)-4-isoxazolyl]methyl}oxy)phenyl]-1H-benzimidazole-4-carboxylic acid;
and pharmaceutically acceptable salts thereof.

21. 6-[4-({[3-(2,6-Dichlorophenyl)-5-(1-methylethyl)-4-isoxazolyl]methyl}oxy)phenyl]-1H-indole-3-carboxylic acid or a pharmaceutically acceptable salt thereof.

22. 5-[4-({[3-(2,6-Dichlorophenyl)-5-(1-methylethyl)-4-isoxazolyl]methyl}oxy)phenyl]-1H-indole-2-carboxylic acid or a pharmaceutically acceptable salt thereof.

23. A pharmaceutical composition comprising a compound according to claim 1 and a pharmaceutically acceptable carrier or diluent.

24.-26. (canceled)

27. A method for the treatment of cholestatic liver disease in a mammal in need thereof comprising administering to said subject a therapeutically effective amount of a compound according to claim 1.

28. A method for the treatment of organ fibrosis in a mammal in need thereof comprising administering to said subject a therapeutically effective amount of a compound according to claim 1.

29. A method for the treatment of liver fibrosis in a mammal in need thereof, said method comprising administering to said subject a therapeutically effective amount of a compound according to claim 1.

30. (canceled)

31. A method for the treatment of cholestatic liver disease in a mammal in need thereof comprising administering to said subject a therapeutically effective amount of 6-[4-({[3-(2,6-dichlorophenyl)-5-(1-methylethyl)-4-isoxazolyl]methyl}oxy)phenyl]-1H-indole-3-carboxylic acid or a pharmaceutically acceptable salt thereof.

32.-46. (canceled)
